(19) United States
(12) Patent Application Publication
(10) Pub. No.: US 2023/0060292 A1
(43) Pub. Date: Mar. 2, 2023

(54) SPLIT CHIMERIC ANTIGEN RECEPTORS AND METHODS OF USE

(71) Applicant: Kite Pharma, Inc., Santa Monica, CA (US)

(72) Inventor: Christopher J. Bond, Mercer Island, WA (US)

(21) Appl. No.: 17/814,811

(22) Filed: Jul. 25, 2022

Related U.S. Application Data
(60) Provisional application No. 63/225,643, filed on Jul. 26, 2021.

(51) Int. Cl. C07K 14/725; C07K 16/28

(52) U.S. Cl. CPC C07K 14/7051; C07K 16/2803; C07K 16/2887; C07K 2319/33; A61K 38/00

(57) ABSTRACT

Provided herein are cells, such as iNKT cells that include a split dual targeting chimeric antigen receptors (CARs), and methods of use. The split CARs are each linked to an invariant TCR alpha or TRC beta chain.

### CROSS-REFERENCE TO RELATED APPLICATIONS

The present application claims the priority benefit of U.S. Provisional Application No. 63/225,643, filed Jul. 26, 2021, which is hereby incorporated by reference in its entirety.

### SEQUENCE LISTING

The instant application contains a Sequence Listing which has been submitted electronically in XML file format and is hereby incorporated by reference in its entirety. Said XML copy, created on Jul. 25, 2022, is named K-1092-US-NP_SL.xml and is 280,611 bytes in size

### TECHNICAL FIELD

The present disclosure relates to the field of cell therapy, and more specifically, antibodies, chimeric antigen receptors (CARs), and genetically engineered cells.

### BACKGROUND OF THE DISCLOSURE

Human cancers are by their nature comprised of normal cells that have undergone a genetic or epigenetic conversion to become abnormal cancer cells. In doing so, cancer cells begin to express proteins and other antigens that are distinct from those expressed by normal cells. These aberrant tumor antigens can be used by the body's innate immune system to specifically target and kill cancer cells. However, cancer cells employ various mechanisms to prevent immune cells, such as T and B lymphocytes, from successfully targeting cancer cells.

Current therapies T cell therapies rely on enriched or modified human T cells to target and kill cancer cells in a patient. To increase the ability of T cells to target and kill a particular cancer cell, methods have been developed to engineer T cells to express constructs which direct T cells to a particular target cancer cell. Chimeric antigen receptors (CARs) and engineered T cell receptors (TCRs), which comprise binding domains capable of interacting with a particular tumor antigen, allow T cells to target and kill cancer cells that express the particular tumor antigen. A need exists for additional cell platforms for CARs to target and kill cancer cells.

### SUMMARY

Disclosed are an antigen binding systems comprising (i) a first construct comprising a first chimeric antigen receptor (CAR) linked to a first invariant T-cell receptor (TCR) and (ii) a second construct comprising a second CAR linked to a second invariant TCR. In embodiments, first invariant TCR is an invariant TCRα chain and the second invariant TCR is an invariant TCRβ chain.

In embodiments, the first construct comprises in the order of N-terminus to C-terminus (i) an optional leader peptide, (ii) an invariant TCRα chain, (iii) a linker, and (iv) the first CAR, and wherein the second construct comprises in the order of N-terminus to C-terminus (i) an optional leader peptide, (ii) an invariant TCRβ chain, (iii) a linker, and (iv) the second CAR.

In embodiments, the first construct comprises in the order of N-terminus to C-terminus (i) an optional leader peptide, (ii) the first CAR, (iii) a linker, and (iv) an invariant TCRα chain, and wherein the second construct comprises in the order of N-terminus to C-terminus (i) an optional leader peptide, (ii) the second CAR, (iii) a linker, and (iv) an invariant TCRβ chain. In embodiments, the linker is a cleavable linker (e.g., a P2A or T2A linker).

In embodiments, the first CAR comprises a first binding motif that binds a first antigen and the second CAR comprises a second binding motif that binds a second antigen. In embodiments, the first binding motif and second binding motif are both scFVs.

In embodiments, the first CAR and second CAR both further comprise (i) a hinge, (ii) a transmembrane domain, and (iii) an intracellular domain comprising a costimulatory domain and an activation domain.

In embodiments, the first antigen and the second antigen are selected from the group consisting of 5T4, alphafetoprotein, B cell maturation antigen (BCMA), TACI, CA-125, carcinoembryonic antigen, CD19, CD20, CD22, CD23, CD30, CD33, CD56, CD123, CD138, c-Met, CSPG4, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gp120, IL-11Ralpha, kappa chain, lambda chain, melanoma-associated antigen, mesothelin, MUC-1, mutated p53, mutated ras, prostate-specific antigen, ROR1, VEGFR2, and wherein the first antigen and the second antigen are different.

In embodiments, the first binding motif is a CD19 or a CD20 binding motif and the second binding motif is a CD19 or a CD20 binding motif, and wherein the first binding motif and the second binding motif are different.

In amendments, the CD19 binding motif comprises a first domain comprising three heavy chain complementarity determining regions (CDRH1, CDRH2, and CDRH3) and a second domain comprising three light chain complementarity determining regions (CDRL1, CDRL2, and CDRL3), wherein (i) the CDRH1 has a sequence according to any one of SEQ ID NOs: 223-225; (ii) the CDRH2 has a sequence according to any one of SEQ ID NOs: 226-228; (iii) the CDRH3 has a sequence according to any one of SEQ ID NOs: 229-231; (iv) the CDRL1 has a sequence according to any one of SEQ ID NOs: 234-236; (v) the CDRL2 has a sequence according to any one of SEQ ID NOs: 237-239; and (vi) the CDRL3 has a sequence according to any one of SEQ ID NOs: 240-242; and wherein the CD20 binding motif comprises a first domain comprising three heavy chain complementarity determining regions (CDRH1, CDRH2, and CDRH) and a second domain comprising three light chain complementarity determining regions (CDRL1, CDRL2, and CDRL3), wherein (i) the CDRH1 has a sequence according to any one of SEQ ID NOs: 3-5, 25-27, 47-49, 69-71, 91-93, 113-115, 135-137, 157-159, 179-181, and 201-203; (ii) the CDRH2 has a sequence according to any one of SEQ ID NOs: 6-8, 28-30, 50-52, 72-74, 94-96, 116-118, 138-140, 160-162, 182-184, and 204-206; (iii) the CDRH3 has a sequence according to any one of SEQ ID NOs: 9-11, 31-33, 53-55, 75-77, 96-98, 119-121, 141-143, 163-165, 185-187, and 207-209; (iv) the CDRL1 has a sequence according to any one of SEQ ID NOs: 14-16, 36-38, 58-60, 80-82, 102-104, 124-126, 146-148, 168-170, 190-192, and 212-214; (v) the CDRL2 has a sequence according to any one of SEQ ID NOs: 17-19, 39-41, 61-63, 83-85, 105-107, 127-129, 149-151, 171-173, 193-195, and 215-217; and (vi) the CDRL3 has a sequence according to any one of SEQ ID NOs: 20-22, 42-44, 64-66, 86-88, 108-110, 130-132, 152-154, 174-176, 196-198, and 218-220.

In embodiments, the CD19 binding motif comprises a first domain comprising three heavy chain complementarity determining regions (CDRH1, CDRH2, and CDRH3) and a second domain comprising three light chain complementarity determining regions (CDRL1, CDRL2, and CDRL3), wherein the CDRHs and CDRLs comprise: an CDRH1 according to any of SEQ ID NOs: 223-225; an CDRH2 according to any of SEQ ID NOs: 226-228; an CDRH3 according to any one of SEQ ID NOs: 229-231; an CDRL1 according to any of SEQ ID NOs: 234-236; an CDRL2 according to any of SEQ ID NOs: 237-239; an CDRL3 according to any one of SEQ ID NOs: 240-242; and wherein the CD20 binding motif comprises a first domain comprising three heavy chain complementarity determining regions (CDRH1, CDRH2, and CDRH) and a second domain comprising three light chain complementarity determining regions (CDRL1, CDRL2, and CDRL3), wherein CDRHs and CDRLs comprise: (i) an CDRH1 according to any of SEQ ID NOs: 3-5; an CDRH2 according to any of SEQ ID NOs: 6-8; an CDRH3 according to any one of SEQ ID NOs: 9-11; an CDRL1 according to any of SEQ ID NOs: 14-16; an CDRL2 according to any of SEQ ID NOs: 17-19; an CDRL3 according to any one of SEQ ID NOs: 20-22; (ii) an CDRH1 according to any of SEQ ID NOs: 25-27; an CDRH2 according to any of SEQ ID NOs: 28-30; an CDRH3 according to any one of SEQ ID NOs: 31-33; an CDRL1 according to any of SEQ ID NOs: 36-38; an CDRL2 according to any of SEQ ID NOs: 39-41; an CDRL3 according to any one of SEQ ID NOs: 42-44; (iii) an CDRH1 according to any of SEQ ID NOs: 47-49; an CDRH2 according to any of SEQ ID NOs: 50-52; an CDRH3 according to any one of SEQ ID NOs: 53-55; an CDRL1 according to any of SEQ ID NOs: 58-60; an CDRL2 according to any of SEQ ID NOs: 61-63; an CDRL3 according to any one of SEQ ID NOs: 64-66; (iv) an CDRH1 according to any of SEQ ID NOs: 69-71; an CDRH2 according to any of SEQ ID NOs: 72-74; an CDRH3 according to any one of SEQ ID NOs: 75-77; an CDRL1 according to any of SEQ ID NOs: 80-82; an CDRL2 according to any of SEQ ID NOs: 83-85; an CDRL3 according to any one of SEQ ID NOs: 86-88; (v) an CDRH1 according to any of SEQ ID NOs: 91-93; an CDRH2 according to any of SEQ ID NOs: 94-96; an CDRH3 according to any one of SEQ ID NOs: 96-98; an CDRL1 according to any of SEQ ID NOs: 102-104; an CDRL2 according to any of SEQ ID NOs: 105-107; an CDRL3 according to any one of SEQ ID NOs: 108-110; (vi) an CDRH1 according to any of SEQ ID NOs: 113-115; an CDRH2 according to any of SEQ ID NOs: 116-118; an CDRH3 according to any one of SEQ ID NOs: 119-121; an CDRL1 according to any of SEQ ID NOs: 124-126; an CDRL2 according to any of SEQ ID NOs: 127-129; an CDRL3 according to any one of SEQ ID NOs: 130-132; (vii) an CDRH1 according to any of SEQ ID NOs: 135-137; an CDRH2 according to any of SEQ ID NOs: 138-140; an CDRH3 according to any one of SEQ ID NOs: 141-143; an CDRL1 according to any of SEQ ID NOs: 146-148; an CDRL2 according to any of SEQ ID NOs: 149-151; an CDRL3 according to any one of SEQ ID NOs: 152-154; (viii) an CDRH1 according to any of SEQ ID NOs: 157-159; an CDRH2 according to any of SEQ ID NOs: 160-162; an CDRH3 according to any one of SEQ ID NOs: 163-165; an CDRL1 according to any of SEQ ID NOs: 168-170; an CDRL2 according to any of SEQ ID NOs: 171-173; an CDRL3 according to any one of SEQ ID NOs: 174-176; (ix) an CDRH1 according to any of SEQ ID NOs: 179-181; an CDRH2 according to any of SEQ ID NOs: 182-184; an CDRH3 according to any one of SEQ ID NOs: 185-187; an CDRL1 according to any of SEQ ID NOs: 190-192; an CDRL2 according to any of SEQ ID NOs: 193-195; an CDRL3 according to any one of SEQ ID NOs: 196-198; or (x) an CDRH1 according to any of SEQ ID NOs: 201-203; an CDRH2 according to any of SEQ ID NOs: 204-206; an CDRH3 according to any one of SEQ ID NOs: 207-209; an CDRL1 according to any of SEQ ID NOs: 212-214; an CDRL2 according to any of SEQ ID NOs: 215-217; an CDRL3 according to any one of SEQ ID NOs: 218-220.

In embodiments, the CD19 binding motif comprises a heavy chain variable domain comprising the three CDRHs and a light chain variable domain comprising the three CDRLs, wherein: (i) the heavy chain variable domain is at least 80% identical to SEQ ID NO: 221; and (ii) the light chain variable domain is at least 80% identical to SEQ ID NO: 233; and wherein the CD20 binding motif comprises a heavy chain variable domain comprising the three CDRHs and a light chain variable domain comprising the three CDRLs, wherein: (i) the heavy chain variable domain is at least 80% identical to SEQ ID NOs: 1, 23, 45, 67, 89, 111, 133, 155, 177, or 199; and (ii) the light chain variable domain is at least 80% identical to SEQ ID NOs: 12, 34, 56, 78, 100, 122, 144, 166, 188, or 210.

In embodiments, the CD19 binding motif comprises a first heavy chain variable domain comprising the three CDRHs and a light chain variable domain comprising the three CDRLs, wherein: (i) the heavy chain variable domain is at least 80% identical to SEQ ID NO: 221 and the light chain variable domain is at least 80% identical to SEQ ID NO: 233; and wherein the CD20 binding motif comprises a first heavy chain variable domain comprising the three CDRHs and a light chain variable domain comprising the three CDRLs, wherein: (i) the heavy chain variable domain is at least 80% identical to SEQ ID NO: 1 and the light chain variable domain is at least 80% identical to SEQ ID NO: 12; (ii) the heavy chain variable domain is at least 80% identical to SEQ ID NO: 23 and the light chain variable domain is at least 80% identical to SEQ ID NO: 34; (iii) the heavy chain variable domain is at least 80% identical to SEQ ID NO: 45 and the light chain variable domain is at least 80% identical to SEQ ID NO: 56; (iv) the heavy chain variable domain is at least 80% identical to SEQ ID NO: 67 and the light chain variable domain is at least 80% identical to SEQ ID NO: 78; (v) the heavy chain variable domain is at least 80% identical to SEQ ID NO: 89 and the light chain variable domain is at least 80% identical to SEQ ID NO: 100; (vi) the heavy chain variable domain is at least 80% identical to SEQ ID NO: 111 and the light chain variable domain is at least 80% identical to SEQ ID NO: 122; (vii) the heavy chain variable domain is at least 80% identical to SEQ ID NO: 133 and the light chain variable domain is at least 80% identical to SEQ ID NO: 144; (viii) the heavy chain variable domain is at least 80% identical to SEQ ID NO: 155 and the light chain variable domain is at least 80% identical to SEQ ID NO: 166; (ix) the heavy chain variable domain is at least 80% identical to SEQ ID NO: 177 and the light chain variable domain is at least 80% identical to SEQ ID NO: 188; or (x) the heavy chain variable domain is at least 80% identical to SEQ ID NO: 199 and the light chain variable domain is at least 80% identical to SEQ ID NO: 210.

In embodiments, the antigen binding system is encoded by a vector.

In embodiments, the antigen binding system further comprises (i) a first vector that comprises a nucleic acid encoding the first construct and (ii) a second vector that comprises a nucleic acid encoding the second construct.

Disclosed are nucleic acids encoding antigen binding systems of the present disclosure.

Disclosed are vectors comprising the nucleic acids encoding antigen binding systems of the present disclosure. In embodiments, the vector is a viral vector. In embodiments, an antigen binding system of the present disclosure comprises (i) a first vector comprising a nucleic acid encoding a first chimeric antigen receptor (CAR) linked via a cleavable linker to an invariant TCRα chain and (ii) a second vector comprising a nucleic acid encoding a second CAR linked via a cleavable linker to an invariant TCRβ chain.

Disclosed are cells that comprises antigen binding systems, nucleic acids, and/or vectors of the present disclosure. In embodiments, the cell is an immune cell. In embodiments, the immune cell is selected from the group consisting of a T cell, a B cell, a tumor infiltrating lymphocyte (TIL), a TCR expressing cell, a natural killer (NK) cell, iNKT cell, a dendritic cell, a granulocyte, an innate lymphoid cell, a megakaryocyte, a monocyte, a macrophage, a platelet, a thymocyte, and a myeloid cell.

Disclosed are pharmaceutical compositions comprising the cells of the present disclosure.

Disclosed are methods of generating an engineered cell, the method comprising transfecting or transducing a cell with antigen binding systems, nucleic acids, and/or vectors of the present disclosure. In embodiments, the engineered cell is an autologous cell or an allogeneic cell. In embodiments, the cell transfected or transduced is an HSC or iPSC.

### DETAILED DESCRIPTION OF THE DISCLOSURE

Any aspect or embodiment described herein may be combined with any other aspect or embodiment as disclosed herein. While the present disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims. The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, dictionaries, documents, manuscripts and scientific literature cited herein are hereby incorporated by reference. Other features and advantages of the disclosure will be apparent from the following Detailed Description, comprising the Examples, and the claims.

### Definitions

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2^{nd} ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2^{nd} ed, (2006), Oxford University Press, provide those of skill in the art with a general dictionary for many of the terms used in this disclosure.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

The term “human antibody” is intended to comprise antibodies having variable and constant domain sequences generated, assembled, or derived from human immunoglobulin sequences, or sequences indistinguishable therefrom. In some embodiments, antibodies (or antibody components) may be considered to be “human” even though their amino acid sequences comprise residues or elements not encoded by human germline immunoglobulin sequences (e.g., variations introduced by in vitro random or site-specific mutagenesis or introduced by in vivo somatic mutation). The term “humanized” is intended to comprise antibodies having a variable domain with a sequence derived from a variable domain of a non-human species (e.g., a mouse), modified to be more similar to a human germline encoded sequence. In some embodiments, a “humanized” antibody comprises one or more framework domains having substantially the amino acid sequence of a human framework domain, and one or more complementary determining regions having substantially the amino acid sequence as that of a non-human antibody. In some embodiments, a humanized antibody comprises at least a portion of an immunoglobulin constant region (Fc), generally that of a human immunoglobulin constant domain. In some embodiments, a humanized antibodies may comprise a C_{H}1, hinge, C_{H}2, C_{H}3, and, optionally, a C_{H}4 region of a human heavy chain constant domain.

Antibodies can include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above. In certain embodiments, antibodies described herein refer to polyclonal antibody populations. Antibodies may also comprise, for example, Fab′ fragments, Fd′ fragments, Fd fragments, isolated CDRs, single chain Fvs, polypeptide-Fc fusions, single domain antibodies (e.g., shark single domain antibodies such as IgNAR or fragments thereof), camelid antibodies, single chain or Tandem diabodies (TandAb®), Anticalins®, Nanobodies® minibodies, BiTE®s, ankyrin repeat proteins or DARPINs®, Avimers®, DARTs, TCR-like antibodies, Adnectins®, Affilins®, Trans-bodies®, Affibodies®, TrimerX®, MicroProteins, Fynomers®, Centyrins®, and KALBITOR®s.

An immunoglobulin may derive from any of the commonly known isotypes, including but not limited to IgA, secretory IgA, IgG, IgE and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “antigen binding molecule,” “antigen binding portion,” “antigen binding domain,” or “antibody fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments, the antigen binding molecule binds to CD19 or CD20. In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv).

In some instances, a CDR is substantially identical to one found in a reference antibody (e.g., an antibody of the present disclosure) and/or the sequence of a CDR provided in the present disclosure. In some embodiments, a CDR is substantially identical to a reference CDR (e.g., a CDR provided in the present disclosure) in that it is either identical in sequence or contains between 1, 2, 3, 4, or 5 (e.g., 1-5) amino acid substitutions as compared with the reference CDR. In some embodiments a CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR (e.g., 85-90%, 85-95%, 85-100%, 90-95%, 90-100%, or 95-100%). In some embodiments a CDR is substantially identical to a reference CDR in that it shows at least 96%, 96%, 97%, 98%, 99%, or 100% sequence identity with the reference CDR. In some embodiments a CDR is substantially identical to a reference CDR in that one amino acid within the CDR is deleted, added, or substituted as compared with the reference CDR while the CDR has an amino acid sequence that is otherwise identical with that of the reference CDR. In some embodiments a CDR is substantially identical to a reference CDR in that 2, 3, 4, or 5 (e.g., 2-5) amino acids within the CDR are deleted, added, or substituted as compared with the reference CDR while the CDR has an amino acid sequence that is otherwise identical to the reference CDR. In various embodiments, an antigen binding fragment binds a same antigen as a reference antibody.

An antigen binding fragment may be produced by any means. For example, in some embodiments, an antigen binding fragment may be enzymatically or chemically produced by fragmentation of an intact antibody. In some embodiments, an antigen binding fragment may be recombinantly produced (i.e., by expression of an engineered nucleic acid sequence. In some embodiments, an antigen binding fragment may be wholly or partially synthetically produced. In some embodiments, an antigen binding fragment an antigen-binding fragment) may have a length of at least about 50, 60, 70, 80, 90, 100, 110, 120, 130, 140, 150, 160, 170, 180, 190 amino acids or more; in some embodiments at least about 200 amino acids (e.g., 50-100, 50-150, 50-200, or 100-200 amino acids).

The term “variable region” or “variable domain” is used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

TABLE 1
CDR Numbering

| Loop | Kabat | AbM | Chothia | Contact |
|---|---|---|---|---|
| L1 | L24--L34 | L24--L34 | L24--L34 | L30--L36 |
| L2 | L50--L56 | L50--L56 | L50--L56 | L46--L55 |
| L3 | L89--L97 | L89--L97 | L89--L97 | L89--L96 |
| H1 | H31--H35B | H26--H35B | H26--H32 . . . 34 | H30--H35B |
| | (Kabat Numbering) | | | |
| H1 | H31--H35 | H26--H35 | H26--H32 | H30--H35 |
| | (Chothia Numbering) | | | |
| H2 | H50--H65 | H50--H58 | H52--H56 | H47--H58 |
| H3 | H95--H102 | H95--H102 | H95--H102 | H93--H101 |

The term “Kabat numbering” and like terms are recognized in the art and refer to a system of numbering amino acid residues in the heavy and light chain variable regions of an antibody, or an antigen-binding molecule thereof. In certain aspects, the CDRs of an antibody can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

In certain aspects, the CDRs of an antibody can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). In a specific embodiment, the CDRs of the antibodies described herein have been determined according to the Chothia numbering scheme.

The terms “constant region” and “constant domain” are interchangeable and have a meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

The term “heavy chain” when used in reference to an antibody can refer to any distinct type, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

The term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as BIACORE® or KinExA.

A “conservative amino acid substitution” is one in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody or antigen-binding molecule thereof can be replaced with an amino acid residue with a similar side chain. In general, two sequences are generally considered to be “substantially similar” if they contain a conservative amino acid substitution in corresponding positions. For example, certain amino acids are generally classified as “hydrophobic” or “hydrophilic” amino acids, and/or as having “polar” or “non-polar” side chains. Substitution of one amino acid for another of the same type may be considered a conservative substitution. Exemplary amino acid categorizations are summarized in Tables 2 and 3 below:

TABLE 2

| | | | | | Hydropathy |
|---|---|---|---|---|---|
| Amino Acid | 3-Letter | 1-Letter | Property | Property | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

TABLE 3

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

The term “heterologous” means from any source other than naturally occurring sequences. For example, a heterologous nucleotide sequence refers to a nucleotide sequence other than that of the wild type human costimulatory protein-encoding sequence.

An “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody:antigen crystals may be studied using well known X-ray diffraction techniques and may be refined using computer software such as X-PLOR (Yale University, 1992, distributed by Molecular Simulations, Inc.; see e.g., Meth Enzymol (1985) volumes 114 & 115, eds Wyckoff H W et al., U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies may be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

An antigen binding molecule, an antibody, or an antigen binding fragment thereof “cross-competes” with a reference antibody or an antigen binding fragment thereof if the interaction between an antigen and the first antigen binding molecule, antibody, or an antigen binding fragment thereof blocks, limits, inhibits, or otherwise reduces the ability of the reference antigen binding molecule, reference antibody, or an antigen binding fragment thereof to interact with the antigen. Cross competition can be complete, e.g., binding of the antigen binding molecule to the antigen completely blocks the ability of the reference binding molecule to bind the antigen, or it can be partial, e.g., binding of the binding molecule to the antigen reduces the ability of the reference binding molecule to bind the antigen. In certain embodiments, an antigen binding molecule that cross-competes with a reference antigen binding molecule binds the same or an overlapping epitope as the reference antigen binding molecule. In other embodiments, the antigen binding molecule that cross-competes with a reference antigen binding molecule binds a different epitope as the reference antigen binding molecule. Numerous types of competitive binding assays can be used to determine if one antigen binding molecule competes with another, for example: solid phase direct or indirect radioimmunoassay (RIA); solid phase direct or indirect enzyme immunoassay (EIA); sandwich competition assay (Stahli et al., 1983, Methods in Enzymology 9:242-253); solid phase direct biotin-avidin EIA (Kirkland et al., 1986, J. Immunol. 137:3614-3619); solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15); solid phase direct biotin-avidin EIA (Cheung, et al., 1990, Virology 176:546-552); and direct labeled RIA (Moldenhauer et al., 1990, Scand. J. Immunol. 32:77-82).

The term “binding” generally refers to a non-covalent association between or among two or more entities. Direct binding involves physical contact between entities or moieties. “Indirect” binding involves physical interaction by way of physical contact with one or more intermediate entities. Binding between two or more entities may be assessed in any of a variety of contexts, e.g., where interacting entities or moieties are studied in isolation or in the context of more complex systems (e.g., while covalently or otherwise associated with a carrier entity and/or in a biological system such as a cell).

The terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen may bind to other peptides or polypeptides, generally with lower affinity as determined by, e.g., immunoassays, BIACORE®, KinExA 3000 instrument (Sapidyne Instruments, Boise, Id.), or other assays known in the art. In a specific embodiment, molecules that specifically bind to an antigen bind to the antigen with a K_{A} that is at least 2 logs, 2.5 logs, 3 logs, 4 logs or greater than the K_{A} when the molecules bind to another antigen. Binding may comprise preferential association of a antigen binding molecule, an antibody, or an antigen binding fragment thereof, with a target of the antigen binding molecule, an antibody, or an antigen binding fragment thereof, as compared to association of the antigen binding molecule, an antibody, or an antigen binding fragment thereof, with an entity that is not the target (i.e., non-target). In some embodiments, antigen binding molecule, an antibody, or an antigen binding fragment thereof, selectively binds a target if binding between the antigen binding molecule, an antibody, or an antigen binding fragment thereof, and the target is greater than 2-fold, greater than 5-fold, greater than 10-fold, 20-fold, 30-fold, 40-fold, 50-fold, 60-fold, 70-fold, 80-fold, 90-fold, or greater than 100-fold as compared with binding of the antigen binding molecule, an antibody, or an antigen binding fragment thereof, and a non-target. In some embodiments, an antigen binding molecule, an antibody, or an antigen binding fragment thereof, selectively binds a target if the binding affinity is less than about 10⁻⁵ M, less than about 10⁻⁶ M, less than about 10⁻⁷ M, less than about 10⁻⁸ M, or less than about 10⁻⁹ M.

In another embodiment, molecules that specifically bind to an antigen binds with a dissociation constant (K_{d}) of about 1×10⁻⁷ M. In some embodiments, the antigen binding molecule specifically binds an antigen with “high affinity” when the K_{d} is about 1×10⁻⁹ M to about 5×10⁻⁹ M. In some embodiments, the antigen binding molecule specifically binds an antigen with “very high affinity” when the K_{d} is 1×10⁻¹⁰ M to about 5×10⁻¹⁰ M. In one embodiment, the antigen binding molecule has a K_{d} of 10⁻⁹ M. In one embodiment, the off-rate is less than about 1×10⁻⁵. In some embodiments, the antigen binding molecule binds human CD19 or CD20 with a K_{d} of between about 1×10⁻⁷M and about 1×10⁻¹³ M. In yet another embodiment, the antigen binding molecule binds human CD19 or CD20 with a K_{d} of about 1×10⁻¹⁰ M to about 5×10⁻¹⁰ M.

In a specific embodiment, provided herein is an antibody or an antigen binding molecule thereof that binds to a target human antigen with higher affinity than to another species of the target antigen. In some embodiments, provided herein is an antibody or an antigen binding molecule thereof that binds to human CD19, or human CD20 with higher affinity than to another species of one or both target antigens, e.g., a non-human CD19, or non-human CD20. In certain embodiments, provided herein is an antibody or an antigen binding molecule thereof that binds to human CD19, or human CD20 with a 5%, 10%, 15%, 20%, 25%, 30%, 35%, 40%, 45%, 50%, 55%, 60%, 65%, 70% or higher affinity than to another species of one or both target antigens as measured by, e.g., a radioimmunoassay, surface plasmon resonance, or kinetic exclusion assay. In a specific embodiment, an antibody or an antigen binding molecule thereof described herein, which binds to a target human antigen, will bind to another species of the target antigen with less than 10%, 15%, or 20% of the binding of the antibody or an antigen binding molecule thereof to the human antigen as measured by, e.g., a radioimmunoassay, surface plasmon resonance, or kinetic exclusion assay.

“Chimeric antigen receptor” or “CAR” refers to a molecule engineered to comprise a binding motif and a means of activating immune cells (for example T cells such as naive T cells, central memory T cells, effector memory T cells, invariant natural killer (iNKT) cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. “Extracellular domain” (or “ECD”) refers to a portion of a polypeptide that, when the polypeptide is present in a cell membrane, is understood to reside outside of the cell membrane, in the extracellular space.

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, can serve as an antigen. An antigen can be endogenously expressed, i.e., expressed by genomic DNA, or can be recombinantly expressed. An antigen can be specific to a certain tissue, such as a cancer cell, or it can be broadly expressed. In addition, fragments of larger molecules can act as antigens. In one embodiment, antigens are tumor antigens. In one particular embodiment, the antigen is all or a fragment of CD19 or CD20. A “target” is any molecule bound by a binding motif, antigen binding system, or binding agent, e.g., an antibody. In some embodiments, a target is an antigen or epitope of the present disclosure.

The term “neutralizing” refers to an antigen binding molecule, scFv, antibody, or a fragment thereof, that binds to a ligand and prevents or reduces the biological effect of that ligand. In some embodiments, the antigen binding molecule, antibody, or a fragment thereof, e.g., scFv, directly blocking a binding site on the ligand or otherwise alters the ligand's ability to bind through indirect means (such as structural or energetic alterations in the ligand). In some embodiments, the antigen binding molecule, antibody, or a fragment thereof, e.g., scFv, prevents the protein to which it is bound from performing a biological function.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T cell or iNKT transplantation.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Transformation” refers to any process by which exogenous DNA is introduced into a host cell. Transformation may occur under natural or artificial conditions using various methods. Transformation may be achieved using any known method for the insertion of foreign nucleic acid sequences into a prokaryotic or eukaryotic host cell. In some embodiments, some transformation methodology is selected based on the host cell being transformed and/or the nucleic acid to be inserted. Methods of transformation may comprise, yet are not limited to, viral infection, electroporation, and lipofection. In some embodiments, a “transformed” cell is stably transformed in that the inserted DNA is capable of replication either as an autonomously replicating plasmid or as part of the host chromosome. In some embodiments, a transformed cell may express introduced nucleic acid.

“Vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference for any purpose.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of the present disclosure include, but are not limited to, cancers of the immune system including lymphoma, leukemia, myeloma, and other leukocyte malignancies. In some embodiments, the methods of the present disclosure can be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers. In one particular embodiment, the cancer is multiple myeloma. The particular cancer can be responsive to chemo- or radiation therapy or the cancer can be refractory. A refractory cancer refers to a cancer that is not amendable to surgical intervention and the cancer is either initially unresponsive to chemo- or radiation therapy or the cancer becomes unresponsive over time. Cancer further includes relapsed or refractory large B-cell lymphoma after two or more lines of systemic therapy, including diffuse large B-cell lymphoma (DLBCL) not otherwise specified, primary mediastinal large B-cell lymphoma after two or more lines of systemic therapy, high grade B-cell lymphoma, and DLBCL arising from follicular lymphoma.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

A “cytokine,” as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

A “therapeutically effective amount,” “effective dose,” “effective amount,” or “therapeutically effective dosage” of a therapeutic agent, e.g., engineered CAR T cells and engineered iNKT cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “lymphocyte” includes natural killer (NK) cells, invariant natural killer T (iNKT) cells, T cells, or B cells. NK cells are a type of cytotoxic (cell toxic) lymphocyte that represent a major component of the inherent immune system. NK cells reject tumors and cells infected by viruses. Invariant natural killer T (iNKT) cells are a small population of αβ T lymphocytes highly conserved from mice to human. It works through the process of apoptosis or programmed cell death. They were termed “natural killers” because they do not require activation in order to kill cells. T-cells play a major role in cell-mediated-immunity (no antibody involvement). Its T-cell receptors (TCR) differentiate themselves from other lymphocyte types. The thymus, a specialized organ of the immune system, is primarily responsible for the T cell's maturation. There are six types of T-cells, namely: Helper T-cells (e.g., CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory T_{SCM} cells, like naive cells, are CD45RO−, CCR7+, CD45RA+, CD62L+(L-selectin), CD27+, CD28+ and IL-7Rα+, but they also express large amounts of CD95, IL-2Rβ, CXCR3, and LFA-1, and show numerous functional attributes distinctive of memory cells); (ii) central memory T_{CM} cells express L-selectin and the CCR7, they secrete IL-2, but not IFNγ or IL-4, and (iii) effector memory T_{EM} cells, however, do not express L-selectin or CCR7 but produce effector cytokines like IFNγ and IL-4), Regulatory T-cells (Tregs, suppressor T cells, or CD4+CD25+ regulatory T cells), Natural Killer T-cells (NKT) and Gamma Delta T-cells. B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

“Linker” (L) or “linker domain” or “linker region” refers to an oligo- or polypeptide region from about 1 to 100 amino acids in length, which links together any of the domains/regions of the CAR of the invention. Linkers may be composed of flexible residues like glycine and serine so that the adjacent protein domains are free to move relative to one another. Longer linkers may be used when it is desirable to ensure that two adjacent domains do not sterically interfere with one another. Linkers may be cleavable or non-cleavable. Use of cleavable linkers allows for two, or more polypetides to be derived from a single poly peptide, for example a CAR and a TRC chain. Examples of cleavable linkers include 2A linkers (for example T2A), 2A-like linkers or functional equivalents thereof and combinations thereof. In some embodiments, the linkers include the picornaviral 2A-like linker, CHYSEL sequences of porcine teschovirus (P2A), virus (T2A) or combinations, variants and functional equivalents thereof. In other embodiments, the linker sequences may comprise Asp-Val/Ile-Glu-X-Asn-Pro-Gly^{(2A)}-Pro^{(2B)} motif, which results in cleavage between the 2A glycine and the 2B proline. In some embodiments, the linker may comprise a linker according to SEQ ID NO: 301. Other linkers will be apparent to those of skill in the art and may be used in connection with alternate embodiments of the invention. By way of example, in some examples, a linker may be used to connect or link a CAR and an invariant TCR chain, such as an invariant TCRα or invariant TCRβ chain. In one example a CAR is linked to an invariant TCRα chain. In another example, CAR is linked to an invariant TCRβ chain. In certain embodiments, one CAR is linked to an invariant TCRα chain and second CAR (in the same cell) is linked to an invariant TCRβ chain. In certain embodiments, an anti-CD19 CAR is linked to an invariant TCRα chain and anti-CD20 CAR (in the same cell) is linked to an invariant TCRβ chain. In certain embodiments, an anti-CD20 CAR is linked to an invariant TCRα chain and anti-CD19 CAR (in the same cell) is linked to an invariant TCRβ chain. A linker may be a portion of a multi-element agent that connects different elements to one another. For example, a polypeptide comprises two or more functional or structural domains may comprise a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. A linker may connect or link together any of the domains/regions of a CAR of the present disclosure. In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length (e.g., 1 to 10, 1 to 20, 1 to 30, 1 to 40, 1 to 50, 1 to 60, 1 to 70, 1 to 80, 1 to 90, 1 to 100, 10 to 20, 10 to 30, 10 to 40, 10 to 50, 10 to 60, 10 to 70, 10 to 80, 10 to 90, or 10 to 100 amino acids in length). In some embodiments, a linker is characterized in that it tends not to adopt a rigid three-dimensional structure, and instead provides flexibility to the polypeptide.

“Single chain variable fragment”, “single-chain antibody variable fragments” or “scFv” antibodies refer to forms of antibodies comprising the variable regions of only the heavy and light chains, connected by a linker peptide.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, or iNKT cell which can either be obtained from a patient or a donor. The cell can be modified to express an exogenous construct, such as, e.g., a chimeric antigen receptor (CAR), T cell receptor (TCR) construct, which is incorporated into the cell's genome. For example, CAR and an invariant TCR chain (such as an invariant TCRα or invariant TCRβ chain) connected by a cleavable linker. Engineering generally comprises manipulation by the hand of man. For example, a polynucleotide is considered to be “engineered” when two or more sequences, that are not linked or connected together in that order in nature, are manipulated by the hand of man to be directly linked or connected to one another in the engineered polynucleotide. In the context of manipulation of cells by techniques of molecular biology, a cell or organism is considered to be “engineered” if it has been manipulated so that its genetic information is altered (e.g., new genetic material not previously present has been introduced, for example by transformation, somatic hybridization, transfection, transduction, or other mechanism, or previously present genetic material is altered or removed, for example by substitution or deletion mutation, or by other protocols). In some embodiments, a binding agent is a modified lymphocyte, e.g., a T cell or iNKT cell, may be obtained from a patient, a donor, and/or manufactured. An engineered cell may be modified to express an exogenous construct, such as, e.g., a chimeric antigen receptor (CAR) linked to a T cell receptor (TCR), is incorporated into the cell's genome. Progeny of an engineered polynucleotide or cell are generally referred to as “engineered” even though the actual manipulation was performed on a prior entity. In some embodiments, “engineered” refers to an entity that has been designed and produced. The term “designed” refers to an agent (i) whose structure is or was selected by the hand of man; (ii) that is produced by a process requiring the hand of man; and/or (iii) that is distinct from natural substances and other known agents.

A “T cell receptor” or “TCR” refers to antigen-recognition molecules present on the surface of T-cells. During normal T-cell development, each of the four TCR genes, α, β, γ, and δ, may rearrange leading to highly diverse TCR proteins. As disclosed herein cells, such iNKT cell are engineered to express modified invariant TCR α and TCR β chains that have each been linked to a CAR, such as an anti-CD19 CAR and an anti-CD20 CAR, respectively.

An “immune response” refers to the action of a cell of the immune system (for example, T lymphocytes, B lymphocytes, natural killer (NK) cells. iNKT cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy can include adoptive T cell therapy, tumor-infiltrating lymphocyte (TIL) immunotherapy, autologous cell therapy, engineered autologous cell therapy (eACT™), and allogeneic T cell transplantation and/or iNKT cell therapy. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. No. 5,728,388, and International Publication No. WO 2008/081035.

The T cells of the immunotherapy can come from any source known in the art. For example, iNKT cells can be differentiated in vitro from a hematopoietic stem cell population, or cells can be obtained from a subject. iNKT cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the iNKT cells can be derived from one or more iNKT cell lines available in the art. iNKT cells can also be obtained from a unit of blood collected from a subject using any number of techniques known to the skilled artisan, such as FICOLL™ separation and/or apheresis.

A “patient” includes any human who is afflicted with a cancer (e.g., a lymphoma or a leukemia). The terms “subject” and “patient” are used interchangeably herein.

The term “in vitro” refers to events occurring in an artificial environment, e.g., in a test tube, reaction vessel, cell culture, etc., rather than within a multi-cellular organism. The term “in vitro cell” refers to any cell which is cultured ex vivo. In particular, an in vitro cell can include a T cell. The term “in vivo” refers to events that occur within a multi-cellular organism, such as a human or a non-human animal.

“Antigen presenting cell” or “APC” refers to cells that process and present antigens to T-cells. Exemplary APCs comprise dendritic cells, macrophages, B cells, certain activated epithelial cells, and other cell types capable of TCR stimulation and appropriate T cell costimulation.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a cell, that specifically binds with a cognate stimulatory ligand present on an antigen present cell. A “stimulatory ligand” is a ligand that when present on an antigen presenting cell (e.g., an APC, a dendritic cell, a B-cell, and the like) can specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody (such as OKT3), an MHC Class I molecule loaded with a peptide, a superagonist anti-CD2 antibody, and a superagonist anti-CD28 antibody.

A “costimulatory signal,” as used herein, refers to a signal, which in combination with a primary signal, such as TCR/CD3 ligation, leads to a T cell response, such as, but not limited to, proliferation and/or upregulation or down regulation of key molecules.

A “costimulatory ligand” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-1a, CD1-1b, CD1-1c, CD1-1d, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

The terms “improve,” “increase,” “inhibit,” and “reduce” indicate values that are relative to a baseline or other reference measurement. In some embodiments, an appropriate reference measurement may comprise a measurement in certain system (e.g., in a single individual) under otherwise comparable conditions absent presence of (e.g., prior to and/or after) an agent or treatment, or in presence of an appropriate comparable reference agent. In some embodiments, an appropriate reference measurement may comprise a measurement in comparable system known or expected to respond in a comparable way, in presence of the relevant agent or treatment.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “agent” may refer to a molecule or entity of any class comprising, or a plurality of molecules or entities, any of which may be, for example, a polypeptide, nucleic acid, saccharide, lipid, small molecule, metal, cell, or organism (for example, a fraction or extract thereof) or component thereof. In some embodiments, an agent may be utilized in isolated or pure form. In some embodiments, an agent may be utilized in a crude or impure form. In some embodiments, an agent may be provided as a population, collection, or library, for example that may be screened to identify or characterize members present therein.

Two events or entities are “associated” with one another if the presence, level, and/or form of one is correlated with that of the other. For example, an entity (e.g., polypeptide, genetic signature, metabolite, microbe, etc.) is considered to be associated with a disease, disorder, or condition, if its presence, level, and/or form correlates with incidence of and/or susceptibility to the disease, disorder, or condition (e.g., across a relevant population). For example, two or more entities are physically “associated” with one another if they interact, directly or indirectly, so that they are and/or remain in physical proximity with one another (e.g., bind). In additional examples, two or more entities that are physically associated with one another are covalently linked or connected to one another, or non-covalently associated, for example by means of hydrogen bonds, van der Waals interaction, hydrophobic interactions, magnetism, and combinations thereof.

Term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

To calculate percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span,” as determined by the algorithm). In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm. Other algorithms are also available for comparison of amino acid or nucleic acid sequences, comprising those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs,” Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying similar sequences, the programs mentioned above generally provide an indication of the degree of similarity. In some embodiments, two sequences are considered to be substantially similar if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are similar and/or identical over a relevant stretch of residues (e.g., 85-90%, 85-95%, 85-100%, 90-95%, 90-100%, or 95-100%). In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues. Sequences with substantial sequence similarity may be homologs of one another.

“Combination therapy” refers to those situations in which a subject is simultaneously exposed to two or more therapeutic regimens (e.g., two or more therapeutic moieties). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

“Corresponding to” may be used to designate the position/identity of a structural element in a molecule or composition through comparison with an appropriate reference molecule or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, for purposes of simplicity, residues in a polypeptide may be designated using a canonical numbering system based on a reference related polypeptide, so that an amino acid “corresponding to” a residue at position 100, for example, need not actually be the 100th amino acid in an amino acid chain provided it corresponds to the residue found at position 100 in the reference polypeptide. Various sequence alignment strategies are available, comprising software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that may be utilized, for example, to identify “corresponding” residues in polypeptides and/or nucleic acids in accordance with the present disclosure.

The term “domain” refers to a portion of an entity. In some embodiments, a “domain” is associated with a structural and/or functional feature of the entity, e.g., so that, when the domain is physically separated from the rest of its parent entity, it substantially or entirely retains the structural and/or functional feature. In some embodiments, a domain may comprise a portion of an entity that, when separated from that (parent) entity and linked or connected with a different (recipient) entity, substantially retains and/or imparts on the recipient entity one or more structural and/or functional features, e.g., that characterized it in the parent entity. In some embodiments, a domain is a portion of a molecule (e.g., a small molecule, carbohydrate, lipid, nucleic acid, or polypeptide). In some embodiments, a domain is a section of a polypeptide; in some such embodiments, a domain is characterized by a structural element (e.g., an amino acid sequence or sequence motif, α-helix character, β-sheet character, coiled-coil character, random coil character, etc.), and/or by a functional feature (e.g., binding activity, enzymatic activity, folding activity, signaling activity, etc.).

The term “dosage form” may be used to refer to a physically discrete unit of an active agent (e.g., an antigen-binding system or antibody) for administration to a subject. Generally, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population. The total amount of a therapeutic composition or agent administered to a subject is determined by one or more medical practitioners and may involve administration of more than one dosage forms.

The term “dosing regimen” may be used to refer to a set of one or more unit doses that are administered individually to a subject. In some embodiments, a given therapeutic agent has a recommended dosing regimen, which may involve one or more doses. In some embodiments, a dosing regimen comprises a plurality of doses each of which is separated in time from other doses. In some embodiments, a dosing regimen comprises a plurality of doses and consecutive doses are separated from one another by time periods of equal length; in some embodiments, a dosing regimen comprises a plurality of doses and consecutive doses are separated from one another by time periods of at least two different lengths. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen is periodically adjusted to achieve a desired or beneficial outcome.

“Effector function” refers to a biological result of interaction of an antibody Fc region with an Fc receptor or ligand. Effector functions comprise, without limitation, antibody-dependent cell-mediated cytotoxicity (ADCC), antibody-dependent cell-mediated phagocytosis (ADCP), and complement-mediated cytotoxicity (CMC). An effector function may be antigen binding dependent, antigen binding independent, or both. ADCC refers to lysis of antibody-bound target cells by immune effector cells. Without wishing to be bound by any theory, ADCC is generally understood to involve Fc receptor (FcR)-bearing effector cells recognizing and subsequently killing antibody-coated target cells (e.g., cells that express on their surface antigens to which an antibody is bound). Effector cells that mediate ADCC may comprise immune cells, comprising yet not limited to, one or more of natural killer (NK) cells, macrophages, neutrophils, eosinophils.

“Effector cell” refers to a cell of the immune system that expresses one or more Fc receptors and mediates one or more effector functions. In some embodiments, effector cells may comprise, without limitation, one or more of monocytes, macrophages, neutrophils, dendritic cells, eosinophils, mast cells, platelets, large granular lymphocytes, Langerhans' cells, natural killer (NK) cells, iNKT cells, T-lymphocytes, and B-lymphocytes. Effector cells may be of any organism comprising, without limitation, humans, mice, rats, rabbits, and monkeys.

The term “excipient” refers to an agent that may be comprised in a composition, for example to provide or contribute to a desired consistency or stabilizing effect. In some embodiments, a suitable excipient may comprise, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

A “fragment” or “portion” of a material or entity as described herein has a structure that comprises a discrete portion of the whole, e.g., of a physical entity or abstract entity. In some embodiments, a fragment lacks one or more moieties found in the whole. In some embodiments, a fragment consists of or comprises a characteristic structural element, domain or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer (e.g., 85-90%, 85-95%, 85-100%, 90-95%, 90-100%, or 95-100%). The whole material or entity may in some embodiments be referred to as the “parent” of the fragment.

The term “fusion polypeptide” or “fusion protein” generally refers to a polypeptide comprising at least two segments. Generally, a polypeptide containing at least two such segments is considered to be a fusion polypeptide if the two segments are moieties that (1) are not comprised in nature in the same peptide, and/or (2) have not previously been linked or connected to one another in a single polypeptide, and/or (3) have been linked or connected to one another through action of the hand of man.

The term “gene product” or “expression product” generally refers to an RNA transcribed from the gene (pre- and/or post-processing) or a polypeptide (pre- and/or post-modification) encoded by an RNA transcribed from the gene.

The term “isolated” refers to a substance that (1) has been separated from at least some components with which it was associated at an earlier time or with which the substance would otherwise be associated, and/or (2) is present in a composition that comprises a limited or defined amount or concentration of one or more known or unknown contaminants. An isolated substance, in some embodiments, may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% (e.g., 85-90%, 85-95%, 85-100%, 90-95%, 90-100%, or 95-100%) of other non-substance components with which the substance was associated at an earlier time, e.g., other components or contaminants with which the substance was previously or otherwise would be associated. In certain instances, a substance is isolated if it is present in a composition that comprises a limited or reduced amount or concentration of molecules of a same or similar type. For instance, in certain instances, a nucleic acid, DNA, or RNA substance is isolated if it is present in a composition that comprises a limited or reduced amount or concentration of non-substance nucleic acid, DNA, or RNA molecules. For instance, in certain instances, a polypeptide substance is isolated if it is present in a composition that comprises a limited or reduced amount or concentration of non-substance polypeptide molecules. In certain embodiments, an amount may be, e.g., an amount measured relative to the amount of a desired substance present in a composition. In certain embodiments, a limited amount may be an amount that is no more than 100% of the amount of substance in a composition, e.g., no more than 1%, 5%, 10%, 20%, 30%, 40%, 50%, 60%, 70%, 80%, 90%, or 95% of the amount of substance in a composition (e.g., 85-90%, 85-95%, 85-100%, 90-95%, 90-100%, or 95-100%). In certain instances, a composition is pure or substantially pure with respect to a selected substance. In some embodiments, an isolated substance is about 80%, about 85%, about 90%, about 91%, about 92%, about 93%, about 94%, about 95%, about 96%, about 97%, about 98%, about 99%, or more than about 99% pure (e.g., 85-90%, 85-95%, 85-100%, 90-95%, 90-100%, or 95-100%). A substance is “pure” if it is substantially free of other components or of contaminants. In some embodiments, a substance may still be considered “isolated” or even “pure,” after having been combined with certain other components such as, for example, one or more carriers or excipients (e.g., buffer, solvent, water, etc.); in such embodiments, percent isolation or purity of the substance is calculated without comprising such carriers or excipients.

“Nucleic acid” refers to any polymeric chain of nucleotides. A nucleic acid may be DNA, RNA, or a combination thereof. In some embodiments, a nucleic acid comprises one or more natural nucleic acid residues. In some embodiments, a nucleic acid comprises of one or more nucleic acid analogs. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long (e.g., 20 to 100, 20 to 500, 20 to 1000, 20 to 2000, or 20 to 5000 or more residues). In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide.

“Operably linked” refers to a juxtaposition where the components described are in a relationship permitting them to function in their intended manner. For example, a control element “operably linked” to a functional element is associated in such a way that expression and/or activity of the functional element is achieved under conditions compatible with the control element.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

“Regulatory T cells” (“Treg”, “Treg cells”, or “Tregs”) refer to a lineage of CD4+T lymphocytes that participate in controlling certain immune activities, e.g., autoimmunity, allergy, and response to infection. Regulatory T cells may regulate the activities of T cell populations, and may also influence certain innate immune system cell types. Tregs may be identified by the expression of the biomarkers CD4, CD25 and Foxp3, and low expression of CD127. Naturally occurring Treg cells normally constitute about 5-10% of the peripheral CD4+T lymphocytes. However, Treg cells within a tumor microenvironment (i.e., tumor-infiltrating Treg cells), Treg cells may make up as much as 20-30% of the total CD4+T lymphocyte population.

The term “sample” generally refers to an aliquot of material obtained or derived from a source of interest. In some embodiments, a source of interest is a biological or environmental source. In some embodiments, a source of interest may comprise a cell or an organism, such as a cell population, tissue, or animal (e.g., a human). In some embodiments, a source of interest comprises biological tissue or fluid. In some embodiments, a biological tissue or fluid may comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may comprise an intracellular fluid, an extracellular fluid, an intravascular fluid (blood plasma), an interstitial fluid, a lymphatic fluid, and/or a transcellular fluid. In some embodiments, a biological fluid may comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

The term “stage of cancer” refers to a qualitative or quantitative assessment of the level of advancement of a cancer. In some embodiments, criteria used to determine the stage of a cancer may comprise, without limitation, one or more of where the cancer is located in a body, tumor size, whether the cancer has spread to lymph nodes, whether the cancer has spread to one or more different parts of the body, etc. In some embodiments, cancer may be staged using the so-called TNM System, according to which T refers to the size and extent of the main tumor, usually called the primary tumor; N refers to the number of nearby lymph nodes that have cancer; and M refers to whether the cancer has metastasized. In some embodiments, a cancer may be referred to as Stage 0 (abnormal cells are present without having spread to nearby tissue, also called carcinoma in situ, or CIS; CIS is not cancer, though could become cancer), Stage I-III (cancer is present; the higher the number, the larger the tumor and the more it has spread into nearby tissues), or Stage IV (the cancer has spread to distant parts of the body). In some embodiments, a cancer may be assigned to a stage selected from the group consisting of: in situ; localized (cancer is limited to the place where it started, with no sign that it has spread); regional (cancer has spread to nearby lymph nodes, tissues, or organs): distant (cancer has spread to distant parts of the body); and unknown (there is not enough information to determine the stage).

The phrase “therapeutic agent” may refer to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms or human subjects. In some embodiments, an appropriate population may be defined by various criteria, such as a certain age group, gender, genetic background, preexisting clinical conditions, in accordance with presence or absence of a biomarker, etc. In some embodiments, a therapeutic agent is a substance that may be used to alleviate, ameliorate, relieve, inhibit, prevent, delay onset of, reduce severity of, and/or reduce incidence of one or more symptoms or features of a disease, disorder, and/or condition. In some embodiments, a therapeutic agent is an agent that has been or is required to be approved by a government agency before it may be marketed for administration to humans. In some embodiments, a therapeutic agent is an agent for which a medical prescription is required for administration to humans.

Various aspects of the disclosure are described in further detail below. Among other things, the present disclosure provides methods and compositions useful for treatment of cancer and/or for initiating or modulating immune responses. In certain embodiments, the present disclosure comprises antigen binding systems and binding agents that are dual-targeted in that they comprise a first CAR linked to a first invariant TCR chain, such as an invariant TCRα chain and a second CAR linked to a second invariant TCR chain, such as an invariant TCRβ chain. In some embodiments, a linker, such as a cleavable linker, for example a P2A or T2A, may be used to connect or link the CAR and the invariant TCR chain, such as an invariant TCRα or invariant TCRβ chain. In one example a CAR is linked to an invariant TCRα chain. In another example, CAR is linked to an invariant TCRβ chain. In certain embodiments, one CAR is linked to an invariant TCRα chain and second CAR (in the same cell) is linked to an invariant TCRβ chain. In certain embodiments, an anti-CD19 CAR is linked to an invariant TCRα chain and anti-CD20 CAR (in the same cell) is linked to an invariant TCRβ chain. In certain embodiments, an anti-CD20 CAR is linked to an invariant TCRα chain and anti-CD19 CAR (for example, in the same cell) is linked to an invariant TCRβ chain. In various embodiments, one or more binding domain or motifs of the CARs are scFvs. Exemplary binding motif amino acid sequences, and nucleic acid sequences encoding the same, are provided herein. In some embodiments, the CAR linked to an invariant TCRα chain and the CAR linked to an invariant TCRβ chain are split into two viral vectors. In some embodiments, the CAR linked to an invariant TCRα chain and the CAR linked to an invariant TCRβ chain are in the same viral vectors. In certain embodiments The CAR constructs are separated from the invariant TCR coding sequences (invariant TCRα and invariant TCRβ) by a 2A self-cleaving peptide linker, such as a T2A and/or P2A linker. In certain embodiments, the two constructs have the following architecture: optional leader peptide, coding sequence, linker, coding sequence. For example, in the case of the dual targeting CD19 CD20 constructs examples are shown in table 4. It envisioned that the disclosed CAR TCR fusions can be in either order N-terminal to C-Terminal.

TABLE 4

| Construct | Coding Sequence A | 2A sequence | Coding Sequence B |
|---|---|---|---|
| TCRa CD19 | iTCRa | P2A or T2A | CD19 CAR |
| CD19 TCRa | CD19 CAR | P2A or T2A | iTCRa |
| TCRb CD20 | iTCRb | P2A or T2A | CD20 CAR) |
| CD20 TCRb | CD20 CAR | P2A or T2A | iTCRb |

An example of an invariant TCR alpha chain is Vα24-Jα18. An example of an invariant TCR beta chain is Vβ11. Zhu et al., Cell Stem Cell 25:542-557 (2019).

Various embodiments of the present disclosure provide a viral vector encoding an anti-CD20 CAR/TCR fusion and a viral vector encoding an anti-CD19 CAR/TCR fusion. Various embodiments of the present disclosure provide a viral vector encoding an anti-CD20 CAR/TCR and an anti-CD19 CAR/TCR fusion. Various embodiments of the present disclosure provide binding agent that is a cell encoding or expressing an anti-CD20 CAR/TCR fusion and a viral vector encoding and anti-CD19 CAR/TCR fusion. Various embodiments of the present disclosure provide an iNKT cell engineered to encode or express an anti-CD20 and anti-CD19 chimeric antigen receptor. The present disclosure provides immune cells genetically modified with an integrated gene, e.g., a nucleotide sequence of interest (e.g., a constitutive expression construct and/or an inducible expression construct that comprises such nucleotide sequence). In some embodiments, the present disclosure provides methods of treating a subject having a tumor, comprising administering to the subject an iNKT cell described herein. In some embodiments, methods further comprise administration of one or more additional therapies (e.g., a second binding agent (e.g., CAR-T cell, CAR-NK cell, TCR-T cell, TIL cell, allogeneic NK cell, and autologous NK cell), an antibody-drug conjugate, an antibody, a bispecific antibody, a T cell-engaging bispecific antibody, an engineered antibody, and/or a polypeptide described herein).

Other features, objects, and advantages of the present disclosure are apparent in the detailed description that follows. It should be understood, however, that the detailed description, while indicating embodiments of the present disclosure, is given by way of illustration only, not limitation.

An anti-CD20 CAR of the present disclosure may comprise antigen-binding sequences as found in an antibody described herein. Unless otherwise indicated, it is to be appreciated the references to CD20 in the present disclosure relate to human CD20. In various embodiments, an anti-CD20 CAR of the present disclosure comprises at least one heavy chain CDR (HCDR) provided herein, e.g., at least one HCDR disclosed in any one of Tables 5-14. In various embodiments, an anti-CD20 CAR of the present disclosure comprises two HCDRs provided herein, e.g., at least two HCDRs disclosed in any one of Tables 5-14. In various embodiments, an anti-CD20 CAR of the present disclosure comprises three HCDRs provided herein, e.g., three HCDRs disclosed in any one of Tables 5-14. In various embodiments, an anti-CD20 CAR of the present disclosure comprises at least one light chain CDR (LCDR) provided herein, e.g., at least one LCDR disclosed in any one of Tables 5-14. In various embodiments, an anti-CD20 CAR of the present disclosure comprises two LCDRs provided herein, e.g., at least two LCDRs disclosed in any one of Tables 5-14. In various embodiments, an anti-CD20 CAR of the present disclosure comprises three LCDRs provided herein, e.g., three LCDRs disclosed in any one of Tables 5-14.

In various embodiments, an anti-CD20 CAR of the present disclosure comprises at least one HCDR provided herein, e.g., at least one HCDR disclosed in any one of Tables 5-14, and at least one LCDR provided herein, e.g., at least one LCDR disclosed in any one of Tables 5-14. In various embodiments, an anti-CD20 CAR of the present disclosure comprises one HCDR provided herein, e.g., at least one HCDR disclosed in any one of Tables 5-14, and one LCDR provided herein, e.g., derived from the same Table of Tables 5-14 as the HCDR(s). In various embodiments, an anti-CD20 CAR of the present disclosure comprises two HCDRs provided herein, e.g., at least two HCDRs disclosed in any one of Tables 5-14, and two LCDRs provided herein, e.g., at least two LCDRs disclosed in any one of Tables 5-14. In various embodiments, an anti-CD20 CAR of the present disclosure comprises two HCDRs provided herein, e.g., at least two HCDRs disclosed in any one of Tables 5-14, and two LCDRs provided herein, e.g., derived from the same Table of Tables 5-14 as the HCDR(s). In various embodiments, an anti-CD20 CAR of the present disclosure comprises three HCDRs provided herein, e.g., three HCDRs disclosed in any one of Tables 5-14, and three LCDRs provided herein, e.g., three LCDRs disclosed in any one of Tables 5-14. In various embodiments, an anti-CD20 CAR of the present disclosure comprises three HCDRs provided herein, e.g., three HCDRs disclosed in any one of Tables 5-14, and three LCDRs derived from the same Table of Tables 5-14 as the HCDR(s).

In various embodiments, an anti-CD20 CAR of the present disclosure comprises at least one heavy chain framework region (heavy chain FR) of a heavy chain variable domain disclosed herein, e.g., at least one heavy chain FR of a heavy chain variable domain disclosed in any one of Tables 5-14. In various embodiments, an anti-CD20 CAR of the present disclosure comprises two heavy chain FRs of a heavy chain variable domain disclosed herein, e.g., at least two heavy chain FRs of a heavy chain variable domain disclosed in any one of Tables 5-14. In various embodiments, an anti-CD20 CAR of the present disclosure comprises three heavy chain FRs of a heavy chain variable domain disclosed herein, e.g., three heavy chain FRs of a heavy chain variable domain disclosed in any one of Tables 5-14.

In various embodiments, an anti-CD20 CAR of the present disclosure comprises at least one light chain FR of a light chain variable domain disclosed herein, e.g., at least one light chain FR of a light chain variable domain disclosed in any one of Tables 5-14. In various embodiments, an anti-CD20 CAR of the present disclosure comprises two light chain FRs of a light chain variable domain disclosed herein, e.g., at least two light chain FRs of a light chain variable domain disclosed in any one of Tables 5-14. In various embodiments, an anti-CD20 CAR of the present disclosure comprises three light chain FRs of a light chain variable domain disclosed herein, e.g., three light chain FRs of a light chain variable domain disclosed in any one of Tables 5-14.

In various embodiments, an anti-CD20 CAR of the present disclosure comprises at least one heavy chain FR of a heavy chain variable domain disclosed herein, e.g., at least one heavy chain FR of a heavy chain variable domain disclosed in any one of Tables 5-14, and at least one light chain FR of a light chain variable domain disclosed herein, e.g., at least one light chain FR of a light chain variable domain disclosed in any one of Tables 5-14. In various embodiments, an anti-CD20 CAR of the present disclosure comprises one heavy chain FR of a heavy chain variable domain disclosed herein, e.g., at least one heavy chain FR of a heavy chain variable domain disclosed in any one of Tables 5-14, and one light chain FR of a light chain variable domain disclosed herein, e.g., derived from the same Table of Tables 5-14 as the heavy chain FR(s). In various embodiments, an anti-CD20 CAR of the present disclosure comprises two heavy chain FRs of a heavy chain variable domain disclosed herein, e.g., at least two heavy chain FRs of a heavy chain variable domain disclosed in any one of Tables 5-14, and two light chain FRs of a light chain variable domain disclosed herein, e.g., at least two light chain FRs of a light chain variable domain disclosed in any one of Tables 5-14. In various embodiments, an anti-CD20 CAR of the present disclosure comprises two heavy chain FRs of a heavy chain variable domain disclosed herein, e.g., at least two heavy chain FRs of a heavy chain variable domain disclosed in any one of Tables 5-14, and two light chain FRs of a light chain variable domain disclosed herein, e.g., derived from the same Table of Tables 5-14 as the heavy chain FR(s). In various embodiments, an anti-CD20 CAR of the present disclosure comprises three heavy chain FRs of a heavy chain variable domain disclosed herein, e.g., three heavy chain FRs of a heavy chain variable domain disclosed in any one of Tables 5-14, and three light chain FRs of a light chain variable domain disclosed herein, e.g., three light chain FRs of a light chain variable domain disclosed in any one of Tables 5-14. In various embodiments, an anti-CD20 CAR of the present disclosure comprises three heavy chain FRs of a heavy chain variable domain disclosed herein, e.g., three light chain FRs of a light chain variable domain disclosed in any one of Tables 5-14, and three light chain FRs derived from the same Table of Tables 5-14 as the heavy chain FR(s).

Exemplary antibody sequences provided in Tables 5-14 are suitable for use in any antibody format, comprising, e.g., a tetrameric antibody, a monospecific antibody, a bispecific antibody, an antigen binding fragment, or a binding motif. Heavy chain variable domains and light chain variable domains and portions thereof provided in Tables 5-14 may be comprised in a binding motif.

In various embodiments, an anti-CD20 CAR of the present disclosure comprises one, two, or three FRs that together or each individually have at least 75% identity (e.g., at least 75%, at least 80%, at least 90%, at least 95%, or 100%, e.g., 85-90%, 85-95%, 85-100%, 90-95%, 90-100%, or 95-100%) to corresponding FR(s) of a heavy chain variable domain of a heavy chain variable domain disclosed in in any one of Tables 5-14. In various embodiments, an anti-CD20 CAR of the present disclosure comprises one, two, or three FRs that together or each individually have at least 75% identity (e.g., at least 75%, at least 80%, at least 90%, at least 95%, or 100%) to corresponding FR(s) of a light chain variable domain of a light chain variable domain disclosed in any one of Tables 5-14.

In various embodiments, an anti-CD20 CAR of the present disclosure comprises at least one heavy chain variable domain having at least 75% sequence identity to a heavy chain variable domain disclosed in any one of Tables 5-14 (e.g., at least 75%, at least 80%, at least 90%, at least 95%, or 100% identity; e.g., 85-90%, 85-95%, 85-100%, 90-95%, 90-100%, or 95-100%). In various embodiments, an anti-CD20 CAR of the present disclosure comprises two heavy chain variable domains each having at least 75% sequence identity to a heavy chain variable domain disclosed in Tables 5-14 (e.g., at least 75%, at least 80%, at least 90%, at least 95%, or 100% identity; e.g., 85-90%, 85-95%, 85-100%, 90-95%, 90-100%, or 95-100%), which heavy chain variable domains may be same or different.

In various embodiments, an anti-CD20 CAR of the present disclosure comprises at least one light chain variable domain having at least 75% sequence identity to a light chain variable domain disclosed in any one of Tables 5-14 (e.g., at least 75%, at least 80%, at least 90%, at least 95%, or 100% identity; e.g., 85-90%, 85-95%, 85-100%, 90-95%, 90-100%, or 95-100%). In various embodiments, an anti-CD20 CAR of the present disclosure comprises two light chain variable domains each having at least 75% sequence identity to a light chain variable domain disclosed in any one of Tables 5-14 (e.g., at least 75%, at least 80%, at least 90%, at least 95%, or 100% identity; e.g., 85-90%, 85-95%, 85-100%, 90-95%, 90-100%, or 95-100%), which light chain variable domains may be same or different.

In various embodiments, an anti-CD20 CAR of the present disclosure comprises at least one heavy chain variable domain having at least 75% sequence identity to a heavy chain variable domain disclosed in any one of Tables 5-14 (e.g., at least 75%, at least 80%, at least 90%, at least 95%, or 100% identity; e.g., 85-90%, 85-95%, 85-100%, 90-95%, 90-100%, or 95-100%) and at least one light chain variable domain having at least 75% sequence identity to a light chain variable domain disclosed in any one of Tables 5-14 (e.g., at least 75%, at least 80%, at least 90%, at least 95%, or 100% identity; e.g., 85-90%, 85-95%, 85-100%, 90-95%, 90-100%, or 95-100%). In certain embodiments, an anti-CD20 CAR of the present disclosure comprises one heavy chain variable domain having at least 75% sequence identity to a heavy chain variable domain disclosed in any one of Tables 5-14 (e.g., at least 75%, at least 80%, at least 90%, at least 95%, or 100% identity; e.g., 85-90%, 85-95%, 85-100%, 90-95%, 90-100%, or 95-100%) and one light chain variable domain having at least 75% sequence identity to a light chain variable domain disclosed in any one of Tables 5-14 (e.g., at least 75%, at least 80%, at least 90%, at least 95%, or 100% identity; e.g., 85-90%, 85-95%, 85-100%, 90-95%, 90-100%, or 95-100%), where the heavy chain variable domain and light chain variable domain are optionally derived from the same Table of Tables 5-14.

In various embodiments, an anti-CD20 CAR of the present disclosure comprises two heavy chain variable domains each having at least 75% sequence identity to a heavy chain variable domain disclosed in Tables 5-14 (e.g., at least 75%, at least 80%, at least 90%, at least 95%, or 100% identity; e.g., 85-90%, 85-95%, 85-100%, 90-95%, 90-100%, or 95-100%) and two light chain variable domains each having at least 75% sequence identity to a light chain variable domain disclosed in Tables 5-14 (e.g., at least 75%, at least 80%, at least 90%, at least 95%, or 100% identity; e.g., 85-90%, 85-95%, 85-100%, 90-95%, 90-100%, or 95-100%), where, in various embodiments, (i) each of the heavy chain variable domains may be same or different; (ii) each of the light chain variable domains may be same or different; (iii) at least one heavy chain variable domain and at least one light chain variable domain may be derived from the same Table of Tables 5-14; or (iv) the two heavy chain variable domains and the two light chain variable domains are all derived from the same Table of Tables 5-14. Each of Tables 5-14 represents the heavy chain variable domain and light chain variable domain sequences of an exemplary antibody, comprising (i) the heavy chain variable domain of the exemplary antibody; (ii) a DNA sequence encoding the heavy chain variable domain (iii) three heavy chain variable domain CDRs of the heavy chain variable domain, according to IMGT, Kabat, and Chothia numbering; (iv) the light chain variable domain of the exemplary antibody; (v) a DNA sequence encoding the light chain variable domain; and (vi) three light chain variable domain CDRs of the light chain variable domain, according to IMGT, Kabat, and Chothia numbering. Information provided in each table provides framework amino acid sequences, as well as nucleotide sequences encoding each CDR amino acid sequence and nucleotide sequences encoding corresponding FR amino acid sequence.

In various embodiments a binding motif may comprise a heavy chain variable domain of the present disclosure (e.g., having at least 75% sequence identity to a heavy chain variable domain of any one of Tables 5-14, e.g., at least 80%, 85%, 90%, 95%, or 100% identity; e.g., 85-90%, 85-95%, 85-100%, 90-95%, 90-100%, or 95-100%), a light chain variable domain of the present disclosure (e.g., having at least 75% sequence identity to a light chain variable domain of any one of Tables 5-14, e.g., at least 80%, 85%, 90%, 95%, or 100% identity; e.g., 85-90%, 85-95%, 85-100%, 90-95%, 90-100%, or 95-100%), and a linker (e.g., a linker according to SEQ ID NO: 247 and/or a linker according to any one of SEQ ID NOs: 293, 294, and 296-300; see, e.g., Whitlow et al. Protein Eng. 1993 November; 6(8):989-95.). In various embodiments a binding motif may comprise a leader sequence, a heavy chain variable domain of the present disclosure (e.g., having at least 75% sequence identity to a heavy chain variable domain of any one of Tables 5-14, e.g., at least 80%, 85%, 90%, 95%, or 100% identity; e.g., 85-90%, 85-95%, 85-100%, 90-95%, 90-100%, or 95-100%), a light chain variable domain of the present disclosure (e.g., having at least 75% sequence identity to a light chain variable domain of any one of Tables 5-14, e.g., at least 80%, 85%, 90%, 95%, or 100% identity; e.g., 85-90%, 85-95%, 85-100%, 90-95%, 90-100%, or 95-100%), and a linker. If provided with an amino acid or nucleotide sequence of a binding motif comprising a heavy chain variable domain of the present disclosure and a light chain variable domain of the present disclosure, the linker joining the two variable domains will be apparent from the sequence in view of the present disclosure. If provided with an amino acid or nucleotide sequence of a binding motif comprising a heavy chain variable domain of the present disclosure and a light chain variable domain of the present disclosure, the leader sequence will be apparent in view of the present disclosure. For the avoidance of doubt, a heavy chain variable domain and a light chain variable domain of the present disclosure may be present in any orientation, e.g., an orientation in which the heavy chain variable domain is C terminal of the light chain variable domain or in which the heavy chain variable domain is N terminal of the light chain variable domain. In various embodiments a binding motif may comprise a linker according to SEQ ID NO: 247 and/or a linker according to any one of SEQ ID NOs: 293, 294, and 296-300 adjacent to one or more additional linkers. Exemplary linkers are provided in Table 16.

In certain embodiments, an anti-CD20 CAR of the present disclosure comprises a binding motif that comprises a heavy chain variable domain of the present disclosure, a light chain variable domain of the present disclosure, and a linker having at least 75% sequence identity to SEQ ID NO: 247 (e.g., at least 75%, at least 80%, at least 90%, at least 95%, or 100% identity; e.g., 85-90%, 85-95%, 85-100%, 90-95%, 90-100%, or 95-100%). In certain embodiments, an anti-CD20 CAR of the present disclosure comprises a binding motif that comprises a linker according to SEQ ID NO: 247 and/or a linker according to any one of SEQ ID NOs: 293, 294, and 296-300. In certain embodiments, an anti-CD20 CAR of the present disclosure comprises a binding motif that comprises a heavy chain variable domain of the present disclosure, a light chain variable domain of the present disclosure, and a leader sequence having at least 75% sequence identity to SEQ ID NO: 245 (e.g., at least 75%, at least 80%, at least 90%, at least 95%, or 100% identity; e.g., 85-90%, 85-95%, 85-100%, 90-95%, 90-100%, or 95-100%). In certain embodiments, an anti-CD20 CAR of the present disclosure comprises a binding motif that comprises a CSF2RA leader sequence according to SEQ ID NO: 245. In certain embodiments, an anti-CD20 CAR of the present disclosure comprises a binding motif that comprises a heavy chain variable domain of the present disclosure, a light chain variable domain of the present disclosure, a linker of the present disclosure, and a leader sequence of the present disclosure. Exemplary nucleotide sequences encoding an anti-CD19 binding motif and components thereof are found in SED ID NOs: 246, and 248. In various embodiments, an anti-CD19 binding motif of the present disclosure has a sequence according to any one of the sequences of SEQ ID NOs: 251-260. Exemplary binding motif sequences are provided in Table 18.

Chimeric antigen receptor of the present disclosure that is based on an exemplary antibody provided herein, such as for example Ab 1, may be provided in any fragment or format, comprising a heavy chain variable domain according to the indicated exemplary antibody and a light chain variable domain according to the indicated exemplary antibody.

TABLE 5
Exemplary Antibody Sequences 1 (Ab1)

| SEQ ID | | |
|---|---|---|
| NO: | Description | Sequence |
| 1 | Heavy Chain | QVQLQQWGAGLLKPSETLSLTCAVYGGSFSGYYW |
| | Variable Domain | SWIRQPPGKGLEWIGEIDHSGSTNYNPSLKSRVTIS |
| | | VDTSKNQFSLKLSSVTAADTAVYYCARGGGSWYS |
| | | NWFDPWGQGTMVTVSS |
| 2 | VH (DNA) | CAGGTGCAGCTACAGCAGTGGGGCGCAGGACTG |
| | | TTGAAGCCTTCGGAGACCCTGTCCCTCACCTGCG |
| | | CTGTCTATGGTGGGTCCTTCAGTGGTTACTACTG |
| | | GAGCTGGATCCGCCAGCCCCCAGGGAAGGGGCT |
| | | GGAGTGGATTGGGGAAATCGACCATAGTGGAAG |
| | | CACCAACTACAACCCGTCCCTCAAGAGTCGAGT |
| | | CACCATATCAGTAGACACGTCCAAGAACCAGTT |
| | | CTCCCTGAAGCTGAGCTCTGTGACCGCCGCGGAC |
| | | ACGGCGGTGTACTACTGCGCCAGAGGTGGAGGA |
| | | AGTTGGTACAGCAACTGGTTCGACCCATGGGGA |
| | | CAGGGTACAATGGTCACCGTCTCCTCA |
| 3 | CDRH1 IMGT | GGSFSGYY |
| | (Prot) | |
| 4 | CDRH1 Kabat | GYYWS |
| | (Prot) | |
| 5 | CDRH1 Chothia | GGSFSG |
| | (Prot) | |
| 6 | CDRH2 IMGT | IDHSGST |
| | (Prot) | |
| 7 | CDRH2 Kabat | EIDHSGSTNYNPSLKS |
| | (Prot) | |
| 8 | CDRH2 Chothia | DHSGS |
| | (Prot) | |
| 9 | CDRH3 IMGT | ARGGGSWYSNWFDP |
| | (Prot) | |
| 10 | CDRH3 Kabat | GGGSWYSNWFDP |
| | (Prot) | |
| 11 | CDRH3 Chothia | GGGSWYSNWFDP |
| | (Prot) | |
| 12 | Light Chain | DIQMTQSPSTLSASVGDRVTITCRASQSISSWLAWY |
| | Variable Domain | QQKPGKAPKLLIYDASSLESGVPSRFSGSGSGTEFT |
| | | LTISSLQPDDFATYYCQQDRSLPPTFGGGTKVEIK |
| 13 | VL (DNA) | GACATCCAGATGACCCAGTCTCCTTCCACCCTGT |
| | | CTGCATCTGTAGGAGACAGAGTCACCATCACTTG |
| | | CCGGGCCAGTCAGAGTATTAGTAGCTGGTTGGC |
| | | CTGGTATCAGCAGAAACCAGGGAAAGCCCCTAA |
| | | GCTCCTGATCTATGATGCCTCCAGTTTGGAAAGT |
| | | GGGGTCCCATCAAGGTTCAGCGGCAGTGGATCT |
| | | GGGACAGAATTCACTCTCACCATCAGCAGCCTG |
| | | CAGCCTGATGATTTTGCAACTTATTACTGCCAGC |
| | | AGGACCGAAGTCTCCCTCCTACTTTTGGCGGAGG |
| | | GACCAAGGTTGAGATCAAA |
| 14 | CDRL1 IMGT | RASQSISSWLA |
| | (Prot) | |
| 15 | CDRL1 Kabat (Prot) | RASQSISSWLA |
| 16 | CDRL1 Chothia | RASQSISSWLA |
| | (Prot) | |
| 17 | CDRL2 IMGT | DASSLES |
| | (Prot) | |
| 18 | CDRL2 Kabat (Prot) | DASSLES |
| 19 | CDRL2 Chothia | DASSLES |
| | (Prot) | |
| 20 | CDRL3 IMGT | QQDRSLPPT |
| | (Prot) | |
| 21 | CDRL3 Kabat (Prot) | QQDRSLPPT |
| 22 | CDRL3 Chothia | QQDRSLPPT |
| | (Prot) | |

TABLE 6
Exemplary Antibody Sequences 2 (Ab2)

| SEQ ID | | |
|---|---|---|
| NO: | Description | Sequence |
| 23 | Heavy Chain | QVQLQQWGAGLLKPSETLSLTCAVYGGSFSGIHW |
| | Variable Domain | NWIRQPPGKGLEWIGDIDTSGSTNYNPSLKSRVTIS |
| | | VDTSKNQFSLKLSSVTAADTAVYYCARLGQESATY |
| | | LGMDVWGQGTTVTVSS |
| 24 | VH (DNA) | CAGGTGCAGCTACAGCAGTGGGGCGCAGGACTG |
| | | TTGAAGCCTTCGGAGACCCTGTCCCTCACCTGCG |
| | | CTGTCTATGGTGGGTCCTTCAGTGGTATCCACTG |
| | | GAACTGGATCCGCCAGCCCCCAGGGAAGGGGCT |
| | | GGAGTGGATTGGGGACATCGACACAAGTGGAAG |
| | | CACCAACTACAACCCGTCCCTCAAGAGTCGAGTC |
| | | ACCATATCCGTAGACACGTCCAAGAACCAGTTCT |
| | | CCCTGAAGCTGAGTTCTGTGACCGCCGCAGACAC |
| | | GGCGGTGTACTACTGCGCCAGATTGGGACAGGA |
| | | GTCAGCCACCTATCTCGGAATGGACGTATGGGGC |
| | | CAGGGAACAACTGTCACCGTCTCCTCA |
| 25 | CDRH1 IMGT | GGSFSGIH |
| | (Prot) | |
| 26 | CDRH1 Kabat | GIHWN |
| | (Prot) | |
| 27 | CDRH1 Chothia | GGSFSG |
| | (Prot) | |
| 28 | CDRH2 IMGT | IDTSGST |
| | (Prot) | |
| 29 | CDRH2 Kabat | DIDTSGSTNYNPSLKS |
| | (Prot) | |
| 30 | CDRH2 Chothia | DTSGS |
| | (Prot) | |
| 31 | CDRH3 IMGT | ARLGQESATYLGMDV |
| | (Prot) | |
| 32 | CDRH3 Kabat | LGQESATYLGMDV |
| | (Prot) | |
| 33 | CDRH3 Chothia | LGQESATYLGMDV |
| | (Prot) | |
| 34 | Light Chain | DIVMTQSPDSLAVSLGERATINCKSSQSVLYSSNNK |
| | Variable Domain | NYLAWYQQKPGQPPKLLIYWASTRESGVPDRFSGS |
| | | GSGTDFTLTISSLQAEDVAVYYCQQLYTYPFTFGG |
| | | GTKVEIK |
| 35 | VL (DNA) | GACATCGTGATGACCCAGTCTCCAGACTCCCTGG |
| | | CTGTGTCTCTGGGCGAGAGGGCCACCATCAACTG |
| | | CAAGTCCAGCCAGAGTGTTTTATACAGCTCCAAC |
| | | AATAAGAACTACTTAGCTTGGTACCAGCAGAAA |
| | | CCAGGACAGCCTCCTAAGCTGCTCATTTACTGGG |
| | | CATCTACCCGGGAATCCGGGGTCCCTGACCGATT |
| | | CAGTGGCAGCGGGTCTGGGACAGATTTCACTCTC |
| | | ACCATCAGCAGCCTGCAGGCTGAAGATGTGGCA |
| | | GTTTATTACTGTCAGCAGCTCTACACCTACCCTTT |
| | | CACTTTTGGCGGAGGGACCAAGGTTGAGATCAA |
| | | A |
| 36 | CDRL1 IMGT | KSSQSVLYSSNNKNYLA |
| | (Prot) | |
| 37 | CDRL1 Kabat (Prot) | KSSQSVLYSSNNKNYLA |
| 38 | CDRL1 Chothia | KSSQSVLYSSNNKNYLA |
| | (Prot) | |
| 39 | CDRL2 IMGT | WASTRES |
| | (Prot) | |
| 40 | CDRL2 Kabat (Prot) | WASTRES |
| 41 | CDRL2 Chothia | WASTRES |
| | (Prot) | |
| 42 | CDRL3 IMGT | QQLYTYPFT |
| | (Prot) | |
| 43 | CDRL3 Kabat (Prot) | QQLYTYPFT |
| 44 | CDRL3 Chothia | QQLYTYPFT |
| | (Prot) | |

TABLE 7
Exemplary Antibody Sequences 3 (Ab3)

| SEQ ID | | |
|---|---|---|
| NO: | Description | Sequence |
| 45 | Heavy Chain | QLQLQESGPGLVKPSETLSLTCTVSGGSISSSSYYW |
| | Variable Domain | GWIRQPPGKGLEWIGSIYYSGSTYYNPSLKSRVTIS |
| | | VDTSKNQFSLKLSSVTAADTAVYYCARETDYSSG |
| | | MGYGMDVWGQGTTVTVSS |
| 46 | VH (DNA) | CAGCTGCAGCTGCAGGAGTCGGGCCCAGGACTG |
| | | GTGAAGCCTTCGGAGACCCTGTCCCTCACCTGCA |
| | | CTGTCTCTGGTGGCTCCATCAGCAGTAGTAGTTA |
| | | CTACTGGGGCTGGATCCGCCAGCCCCCAGGGAA |
| | | GGGGCTGGAGTGGATTGGGAGTATCTATTATAG |
| | | TGGGAGCACCTACTACAACCCGTCCCTCAAGAG |
| | | TCGAGTCACCATATCCGTAGACACGTCCAAGAA |
| | | CCAGTTCTCCCTGAAGCTGAGTTCTGTGACCGCC |
| | | GCAGACACGGCGGTGTACTACTGCGCCAGAGAG |
| | | ACTGACTACAGCAGCGGAATGGGATACGGAATG |
| | | GACGTATGGGGCCAGGGAACAACTGTCACCGTC |
| | | TCCTCA |
| 47 | CDRH1 IMGT | GGSISSSSYY |
| | (Prot) | |
| 48 | CDRH1 Kabat | SSSYYWG |
| | (Prot) | |
| 49 | CDRH1 Chothia | GGSISSSS |
| | (Prot) | |
| 50 | CDRH2 IMGT | IYYSGST |
| | (Prot) | |
| 51 | CDRH2 Kabat | SIYYSGSTYYNPSLKS |
| | (Prot) | |
| 52 | CDRH2 Chothia | YYSGS |
| | (Prot) | |
| 53 | CDRH3 IMGT | ARETDYSSGMGYGMDV |
| | (Prot) | |
| 54 | CDRH3 Kabat | ETDYSSGMGYGMDV |
| | (Prot) | |
| 55 | CDRH3 Chothia | ETDYSSGMGYGMDV |
| | (Prot) | |
| 56 | Light Chain | DIQMTQSPSSLSASVGDRVTITCRASQSINSYLNWY |
| | Variable Domain | QQKPGKAPKLLIYAASSLQSGVPSRFSGSGSGTDFT |
| | | LTISSLQPEDFATYYCQQSLADPFTFGGGTKVEIK |
| 57 | VL (DNA) | GACATCCAGATGACCCAGTCTCCATCCTCCCTGT |
| | | CTGCATCTGTAGGAGACAGAGTCACCATCACTTG |
| | | CCGGGCAAGTCAGAGCATTAACAGCTATTTAAA |
| | | TTGGTATCAGCAGAAACCAGGGAAAGCCCCTAA |
| | | GCTCCTGATCTATGCTGCATCCAGTTTGCAAAGT |
| | | GGGGTCCCATCAAGGTTCAGTGGCAGTGGATCT |
| | | GGGACAGATTTCACTCTCACCATCAGCAGTCTGC |
| | | AACCTGAAGATTTTGCAACTTACTACTGCCAGCA |
| | | AAGCCTCGCCGACCCTTTCACTTTTGGCGGAGGG |
| | | ACCAAGGTTGAGATCAAA |
| 58 | CDRL1 IMGT | RASQSINSYLN |
| | (Prot) | |
| 59 | CDRL1 Kabat | RASQSINSYLN |
| | (Prot) | |
| 60 | CDRL1 Chothia | RASQSINSYLN |
| | (Prot) | |
| 61 | CDRL2 IMGT | AASSLQS |
| | (Prot) | |
| 62 | CDRL2 Kabat | AASSLQS |
| | (Prot) | |
| 63 | CDRL2 Chothia | AASSLQS |
| | (Prot) | |
| 64 | CDRL3 IMGT | QQSLADPFT |
| | (Prot) | |
| 65 | CDRL3 Kabat | QQSLADPFT |
| | (Prot) | |
| 66 | CDRL3 Chothia | QQSLADPFT |
| | (Prot) | |

TABLE 8
Exemplary Antibody Sequences 4 (Ab4)

| SEQ | | |
|---|---|---|
| ID | | |
| NO: | Description | Sequence |
| 67 | Heavy Chain | QVQLVQSGAEVKKPGASVKVSCKASGYTFKEYG |
| | Variable | ISWVRQAPGQGLEWMGWISAYSGHTYYAQKLQG |
| | Domain | RVTMTTDTSTSTAYMELRSLRSDDTAVYYCARG |
| | | PHYDDWSGFIIWFDPWGQGTLVTVSS |
| 68 | VH (DNA) | CAGGTTCAGCTGGTGCAGTCTGGAGCTGAGGTG |
| | | AAGAAGCCTGGGGCCTCAGTGAAGGTCTCCTGC |
| | | AAGGCTTCTGGTTACACCTTTAAAGAATATGGT |
| | | ATCAGCTGGGTGCGACAGGCCCCTGGACAAGGG |
| | | CTTGAGTGGATGGGATGGATCAGCGCTTACAGT |
| | | GGTCACACATACTATGCACAGAAGCTCCAGGGC |
| | | AGAGTCACCATGACCACAGACACATCCACGAGC |
| | | ACAGCCTACATGGAGCTGAGGAGCCTGAGATCT |
| | | GACGACACGGCGGTGTACTACTGCGCCAGAGGG |
| | | CCTCACTACGACGACTGGAGCGGATTTATCATA |
| | | TGGTTCGACCCATGGGGACAGGGTACATTGGTC |
| | | ACCGTCTCCTCA |
| 69 | CDRH1 IMGT | GYTFKEYG |
| | (Prot) | |
| 70 | CDRH1 Kabat | EYGIS |
| | (Prot) | |
| 71 | CDRH1 Chothia | GYTFKE |
| | (Prot) | |
| 72 | CDRH2 IMGT | ISAYSGHT |
| | (Prot) | |
| 73 | CDRH2 Kabat | WISAYSGHTYYAQKLQ |
| | (Prot) | |
| 74 | CDRH2 Chothia | SAYSG |
| | (Prot) | |
| 75 | CDRH3 IMGT | ARGPHYDDWSGFIIWFDP |
| | (Prot) | |
| 76 | CDRH3 Kabat | GPHYDDWSGFIIWFDP |
| | (Prot) | |
| 77 | CDRH3 Chothia | GPHYDDWSGFIIWFDP |
| | (Prot) | |
| 78 | Light Chain | DIQMTQSPSSLSASVGDRVTITCRASQSISSYL |
| | Variable | NWYQQKPGKAPKLLIYAASSLQSGVPSRFSGSG |
| | Domain | SGTDFTLTISSLQPEDFATYYCQQSYRFPPTFG |
| | | QGTKVEIK |
| 79 | VL (DNA) | GACATCCAGATGACCCAGTCTCCATCCTCCCTG |
| | | TCTGCATCTGTAGGAGACAGAGTCACCATCACT |
| | | TGCCGGGCAAGTCAGAGCATTAGCAGCTATTTA |
| | | AATTGGTATCAGCAGAAACCAGGGAAAGCCCCT |
| | | AAGCTCCTGATCTATGCTGCATCCAGTTTGCAA |
| | | AGTGGGGTCCCTTCAAGGTTCAGTGGCAGTGGA |
| | | TCTGGGACAGATTTCACTCTCACCATCAGCAGT |
| | | CTGCAACCTGAAGATTTTGCAACTTACTACTGT |
| | | CAACAGAGTTACAGGTTTCCTCCTACCTTTGGC |
| | | CAAGGGACCAAGGTTGAGATCAAA |
| 80 | CDRL1 IMGT | RASQSISSYLN |
| | (Prot) | |
| 81 | CDRL1 Kabat | RASQSISSYLN |
| | (Prot) | |
| 82 | CDRL1 Chothia | RASQSISSYLN |
| | (Prot) | |
| 83 | CDRL2 IMGT | AASSLQS |
| | (Prot) | |
| 84 | CDRL2 Kabat | AASSLQS |
| | (Prot) | |
| 85 | CDRL2 Chothia | AASSLQS |
| | (Prot) | |
| 86 | CDRL3 IMGT | QQSYRFPPT |
| | (Prot) | |
| 87 | CDRL3 Kabat | QQSYRFPPT |
| | (Prot) | |
| 88 | CDRL3 Chothia | QQSYRFPPT |
| | (Prot) | |

TABLE 9
Exemplary Antibody Sequences 5 (Ab5)

| SEQ ID | | |
|---|---|---|
| NO: | Description | Sequence |
| 89 | Heavy Chain | QVQLQESGPGLVKPSETLSLTCTVSGGSISSPDHYW |
| | Variable Domain | GWIRQPPGKGLEWIGSIYASGSTFYNPSLKSRVTIS |
| | | VDTSKNQFSLKLSSVTAADTAVYYCARETDYSSG |
| | | MGYGMDVWGQGTTVTVSS |
| 90 | VH (DNA) | CAGGTGCAGCTGCAGGAGTCGGGCCCAGGACTG |
| | | GTGAAGCCTTCGGAGACCCTGTCCCTCACCTGCA |
| | | CTGTCTCTGGTGGCTCCATCAGCAGTCCCGACCA |
| | | CTACTGGGGCTGGATCCGCCAGCCCCCAGGGAA |
| | | GGGGCTGGAGTGGATTGGGTCCATCTACGCCAG |
| | | TGGGAGCACCTTCTACAACCCGTCCCTCAAGAGT |
| | | CGAGTCACCATATCCGTAGACACGTCCAAGAAC |
| | | CAGTTCTCCCTGAAGCTGAGCTCTGTGACCGCCG |
| | | CGGACACGGCGGTGTACTACTGCGCCAGAGAGA |
| | | CTGACTACAGCAGCGGAATGGGATACGGAATGG |
| | | ACGTATGGGGCCAGGGAACAACTGTCACCGTCT |
| | | CCTCA |
| 91 | CDRH1 IMGT | GGSISSPDHY |
| | (Prot) | |
| 92 | CDRH1 Kabat | SPDHYWG |
| | (Prot) | |
| 93 | CDRH1 Chothia | GGSISSPD |
| | (Prot) | |
| 94 | CDRH2 IMGT | IYASGST |
| | (Prot) | |
| 95 | CDRH2 Kabat | SIYASGSTFYNPSLKS |
| | (Prot) | |
| 96 | CDRH2 Chothia | YASGS |
| | (Prot) | |
| 97 | CDRH3 IMGT | ARETDYSSGMGYGMDV |
| | (Prot) | |
| 98 | CDRH3 Kabat | ETDYSSGMGYGMDV |
| | (Prot) | |
| 99 | CDRH3 Chothia | ETDYSSGMGYGMDV |
| | (Prot) | |
| 100 | Light Chain | DIQMTQSPSSLSASVGDRVTITCRASQSINSYLNWY |
| | Variable Domain | QQKPGKAPKLLIYAASSLQSGVPSRFSGSGSGTDFT |
| | | LTISSLQPEDFATYYCQQSLADPFTFGGGTKVEIK |
| 101 | VL (DNA) | GACATCCAGATGACCCAGTCTCCATCCTCCCTGT |
| | | CTGCATCTGTAGGAGACAGAGTCACCATCACTT |
| | | GCCGGGCAAGTCAGAGCATTAACAGCTATTTAA |
| | | ATTGGTATCAGCAGAAACCAGGGAAAGCCCCTA |
| | | AGCTCCTGATCTATGCTGCATCCAGTTTGCAAAG |
| | | TGGGGTCCCATCAAGGTTCAGTGGCAGTGGATC |
| | | TGGGACAGATTTCACTCTCACCATCAGCAGTCTG |
| | | CAACCTGAAGATTTTGCAACTTACTACTGCCAGC |
| | | AAAGCCTCGCCGACCCTTTCACTTTTGGCGGAGG |
| | | GACCAAGGTTGAGATCAAA |
| 102 | CDRL1 IMGT | RASQSINSYLN |
| | (Prot) | |
| 103 | CDRL1 Kabat | RASQSINSYLN |
| | (Prot) | |
| 104 | CDRL1 Chothia | RASQSINSYLN |
| | (Prot) | |
| 105 | CDRL2 IMGT | AASSLQS |
| | (Prot) | |
| 106 | CDRL2 Kabat | AASSLQS |
| | (Prot) | |
| 107 | CDRL2 Chothia | AASSLQS |
| | (Prot) | |
| 108 | CDRL3 IMGT | QQSLADPFT |
| | (Prot) | |
| 109 | CDRL3 Kabat | QQSLADPFT |
| | (Prot) | |
| 110 | CDRL3 Chothia | QQSLADPFT |
| | (Prot) | |

TABLE 10
Exemplary Antibody Sequences 6 (Ab6)

| SEQ ID | | |
|---|---|---|
| NO: | Description | Sequence |
| 111 | Heavy Chain | QITLKESGPTLVKPTQTLTLTCTFSGFSLDTEGVGV |
| | Variable Domain | GWIRQPPGKALEWLALIYFNDQKRYSPSLKSRLTIT |
| | | KDTSKNQVVLTMTNMDPVDTAVYYCARDTGYSR |
| | | WYYGMDVWGQGTTVTVSS |
| 112 | VH (DNA) | CAGATCACCTTGAAGGAGTCTGGTCCTACGCTG |
| | | GTGAAACCCACACAGACCCTCACGCTGACCTGC |
| | | ACCTTCTCTGGGTTCTCACTCGACACTGAAGGAG |
| | | TGGGTGTGGGCTGGATCCGTCAGCCCCCAGGAA |
| | | AGGCCCTGGAGTGGCTTGCACTCATTTATTTCAA |
| | | TGATCAAAAGCGCTACAGCCCATCTCTGAAGAG |
| | | CAGGCTCACCATCACCAAGGACACCTCCAAAAA |
| | | CCAGGTGGTCCTTACAATGACCAACATGGACCC |
| | | TGTGGACACGGCGGTGTACTACTGCGCCAGAGA |
| | | CACGGGATACAGCCGATGGTACTACGGCATGGA |
| | | TGTATGGGGCCAGGGAACAACTGTCACCGTCTC |
| | | CTCA |
| 113 | CDRH1 IMGT | GFSLDTEGVG |
| | (Prot) | |
| 114 | CDRH1 Kabat | TEGVGVG |
| | (Prot) | |
| 115 | CDRH1 Chothia | GFSLDTEG |
| | (Prot) | |
| 116 | CDRH2 IMGT | IYFNDQK |
| | (Prot) | |
| 117 | CDRH2 Kabat | LIYFNDQKRYSPSLKS |
| | (Prot) | |
| 118 | CDRH2 Chothia | YFNDQ |
| | (Prot) | |
| 119 | CDRH3 IMGT | ARDTGYSRWYYGMDV |
| | (Prot) | |
| 120 | CDRH3 Kabat | DTGYSRWYYGMDV |
| | (Prot) | |
| 121 | CDRH3 Chothia | DTGYSRWYYGMDV |
| | (Prot) | |
| 122 | Light Chain | DIQMTQSPSSVSASVGDRVTITCRASQGISSWLAW |
| | Variable Domain | YQQKPGKAPKLLIYAASSLQSGVPSRFSGSGSGTD |
| | | FTLTISSLQPEDFATYYCQQAYAYPITFGGGTKVEI |
| | | K |
| 123 | VL (DNA) | GACATCCAGATGACCCAGTCTCCATCTTCCGTGT |
| | | CTGCATCTGTAGGAGACAGAGTCACCATCACTT |
| | | GTCGGGCGAGTCAGGGTATTAGCAGCTGGTTAG |
| | | CCTGGTATCAGCAGAAACCAGGGAAAGCCCCTA |
| | | AGCTCCTGATCTATGCTGCATCCAGTTTGCAAAG |
| | | TGGGGTCCCATCAAGGTTCAGCGGCAGTGGATC |
| | | TGGGACAGATTTCACTCTCACCATCAGCAGCCTG |
| | | CAGCCTGAAGATTTTGCAACTTATTACTGTCAGC |
| | | AGGCATACGCCTACCCTATCACTTTTGGCGGAGG |
| | | GACCAAGGTTGAGATCAAA |
| 124 | CDRL1 IMGT | RASQGISSWLA |
| | (Prot) | |
| 125 | CDRL1 Kabat | RASQGISSWLA |
| | (Prot) | |
| 126 | CDRL1 Chothia | RASQGISSWLA |
| | (Prot) | |
| 127 | CDRL2 IMGT | AASSLQS |
| | (Prot) | |
| 128 | CDRL2 Kabat | AASSLQS |
| | (Prot) | |
| 129 | CDRL2 Chothia | AASSLQS |
| | (Prot) | |
| 130 | CDRL3 IMGT | QQAYAYPIT |
| | (Prot) | |
| 131 | CDRL3 Kabat | QQAYAYPIT |
| | (Prot) | |
| 132 | CDRL3 Chothia | QQAYAYPIT |
| | (Prot) | |

TABLE 11
Exemplary Antibody Sequences 7 (Ab7)

| SEQ ID | | |
|---|---|---|
| NO: | Description | Sequence |
| 133 | Heavy Chain | QVQLQQWGAGLLKPSETLSLTCAVYGGSFEKYY |
| | Variable Domain | WSWIRQPPGKGLEWIGEIYHSGLTNYNPSLKSRVT |
| | | ISVDTSKNQFSLKLSSVTAADTAVYYCARVRYDSS |
| | | DSYYYSYDYGMDVWGQGTTVTVSS |
| 134 | VH (DNA) | CAGGTGCAGCTACAGCAGTGGGGCGCAGGACTG |
| | | TTGAAGCCTTCGGAGACCCTGTCCCTCACCTGCG |
| | | CTGTCTATGGTGGGTCCTTCGAAAAATACTACTG |
| | | GAGCTGGATCCGCCAGCCCCCAGGGAAGGGGCT |
| | | GGAGTGGATTGGGGAAATCTACCATAGTGGACT |
| | | CACCAACTACAACCCGTCCCTCAAGAGTCGAGT |
| | | CACCATATCAGTAGACACGTCCAAGAACCAGTT |
| | | CTCCCTGAAGCTGAGCTCTGTGACCGCCGCGGA |
| | | CACGGCGGTGTACTACTGCGCCAGGGTCAGATA |
| | | CGACAGCAGCGACTCCTACTACTATAGCTACGA |
| | | TTATGGAATGGACGTATGGGGCCAGGGAACAAC |
| | | TGTCACCGTCTCCTCA |
| 135 | CDRH1 IMGT | GGSFEKYY |
| | (Prot) | |
| 136 | CDRH1 Kabat | KYYWS |
| | (Prot) | |
| 137 | CDRH1 Chothia | GGSFEK |
| | (Prot) | |
| 138 | CDRH2 IMGT | IYHSGLT |
| | (Prot) | |
| 139 | CDRH2 Kabat | EIYHSGLTNYNPSLKS |
| | (Prot) | |
| 140 | CDRH2 Chothia | YHSGL |
| | (Prot) | |
| 141 | CDRH3 IMGT | ARVRYDSSDSYYYSYDYGMDV |
| | (Prot) | |
| 142 | CDRH3 Kabat | VRYDSSDSYYYSYDYGMDV |
| | (Prot) | |
| 143 | CDRH3 Chothia | VRYDSSDSYYYSYDYGMDV |
| | (Prot) | |
| 144 | Light Chain | DIVLTQSPDSLAVSLGERATINCKSSQSVLYSSNNK |
| | Variable Domain | NYLAWYQQKPGQPPKLLIYWASSRESGVPDRFSG |
| | | SGSGTDFTLTISSLQAEDVAVYYCQQSYSFPWTFG |
| | | GGTKVEIK |
| 145 | VL (DNA) | GACATCGTGCTGACCCAGTCTCCAGACTCCCTG |
| | | GCTGTGTCTCTGGGCGAGAGGGCCACCATCAAC |
| | | TGCAAGTCCAGCCAGAGTGTTTTATACAGCTCC |
| | | AACAATAAGAACTACTTAGCTTGGTACCAGCAG |
| | | AAACCAGGACAGCCTCCTAAGCTGCTCATTTAC |
| | | TGGGCATCTAGCCGGGAATCCGGGGTCCCTGAC |
| | | CGATTCAGTGGCAGCGGGTCTGGGACAGATTTC |
| | | ACTCTCACCATCAGCAGCCTGCAGGCTGAAGAT |
| | | GTGGCAGTTTATTACTGTCAGCAGTCCTACTCCT |
| | | TCCCTTGGACTTTTGGCGGAGGGACCAAGGTTG |
| | | AGATCAAA |
| 146 | CDRL1 IMGT | KSSQSVLYSSNNKNYLA |
| | (Prot) | |
| 147 | CDRL1 Kabat | KSSQSVLYSSNNKNYLA |
| | (Prot) | |
| 148 | CDRL1 Chothia | KSSQSVLYSSNNKNYLA |
| | (Prot) | |
| 149 | CDRL2 IMGT | WASSRES |
| | (Prot) | |
| 150 | CDRL2 Kabat | WASSRES |
| | (Prot) | |
| 151 | CDRL2 Chothia | WASSRES |
| | (Prot) | |
| 152 | CDRL3 IMGT | QQSYSFPWT |
| | (Prot) | |
| 153 | CDRL3 Kabat | QQSYSFPWT |
| | (Prot) | |
| 154 | CDRL3 Chothia | QQSYSFPWT |
| | (Prot) | |

TABLE 12
Exemplary Antibody Sequences 8 (Ab8)

| SEQ ID | | |
|---|---|---|
| NO: | Description | Sequence |
| 155 | Heavy Chain | QVQLQQWGAGLLKPSETLSLTCAVYGGSFSRYVW |
| | Variable Domain | SWIRQPPGKGLEWIGEIDSSGKTNYNPSLKSRVTIS |
| | | VDTSKNQFSLKLSSVTAADTAVYYCARVRYDSSD |
| | | SYYYSYDYGMDVWGQGTTVTVSS |
| 156 | VH (DNA) | CAGGTGCAGCTACAGCAGTGGGGCGCAGGACTG |
| | | TTGAAGCCTTCGGAGACCCTGTCCCTCACCTGCG |
| | | CTGTCTATGGTGGGTCCTTCAGTCGATACGTATG |
| | | GAGCTGGATCCGCCAGCCCCCAGGGAAGGGGCT |
| | | GGAGTGGATTGGGGAAATCGACTCCAGTGGAAA |
| | | AACCAACTACAACCCGTCCCTCAAGAGTCGAGT |
| | | CACCATATCAGTAGACACGTCCAAGAACCAGTT |
| | | CTCCCTGAAGCTGAGCTCTGTGACCGCCGCGGA |
| | | CACGGCGGTGTACTACTGCGCCAGGGTCAGATA |
| | | CGACAGCAGCGACTCCTACTACTATAGCTACGA |
| | | TTATGGAATGGACGTATGGGGCCAGGGAACAAC |
| | | TGTCACCGTCTCCTCA |
| 157 | CDRH1 IMGT | GGSFSRYV |
| | (Prot) | |
| 158 | CDRH1 Kabat | RYVWS |
| | (Prot) | |
| 159 | CDRH1 Chothia | GGSFSR |
| | (Prot) | |
| 160 | CDRH2 IMGT | IDSSGKT |
| | (Prot) | |
| 161 | CDRH2 Kabat | EIDSSGKTNYNPSLKS |
| | (Prot) | |
| 162 | CDRH2 Chothia | DSSGK |
| | (Prot) | |
| 163 | CDRH3 IMGT | ARVRYDSSDSYYYSYDYGMDV |
| | (Prot) | |
| 164 | CDRH3 Kabat | VRYDSSDSYYYSYDYGMDV |
| | (Prot) | |
| 165 | CDRH3 Chothia | VRYDSSDSYYYSYDYGMDV |
| | (Prot) | |
| 166 | Light Chain | DIVLTQSPDSLAVSLGERATINCKSSQSVLYSSNNK |
| | Variable Domain | NYLAWYQQKPGQPPKLLIYWASSRESGVPDRFSG |
| | | SGSGTDFTLTISSLQAEDVAVYYCQQSYSFPWTFG |
| | | GGTKVEIK |
| 167 | VL (DNA) | GACATCGTGCTGACCCAGTCTCCAGACTCCCTGG |
| | | CTGTGTCTCTGGGCGAGAGGGCCACCATCAACT |
| | | GCAAGTCCAGCCAGAGTGTTTTATACAGCTCCA |
| | | ACAATAAGAACTACTTAGCTTGGTACCAGCAGA |
| | | AACCAGGACAGCCTCCTAAGCTGCTCATTTACTG |
| | | GGCATCTAGCCGGGAATCCGGGGTCCCTGACCG |
| | | ATTCAGTGGCAGCGGGTCTGGGACAGATTTCAC |
| | | TCTCACCATCAGCAGCCTGCAGGCTGAAGATGT |
| | | GGCAGTTTATTACTGTCAGCAGTCCTACTCCTTC |
| | | CCTTGGACTTTTGGCGGAGGGACCAAGGTTGAG |
| | | ATCAAA |
| 168 | CDRL1 IMGT | KSSQSVLYSSNNKNYLA |
| | (Prot) | |
| 169 | CDRL1 Kabat | KSSQSVLYSSNNKNYLA |
| | (Prot) | |
| 170 | CDRL1 Chothia | KSSQSVLYSSNNKNYLA |
| | (Prot) | |
| 171 | CDRL2 IMGT | WASSRES |
| | (Prot) | |
| 172 | CDRL2 Kabat | WASSRES |
| | (Prot) | |
| 173 | CDRL2 Chothia | WASSRES |
| | (Prot) | |
| 174 | CDRL3 IMGT | QQSYSFPWT |
| | (Prot) | |
| 175 | CDRL3 Kabat | QQSYSFPWT |
| | (Prot) | |
| 176 | CDRL3 Chothia | QQSYSFPWT |
| | (Prot) | |

TABLE 13
Exemplary Antibody Sequences 9 (Ab9)

| SEQ ID | | |
|---|---|---|
| NO: | Description | Sequence |
| 177 | Heavy Chain | QVQLQQWGAGLLKPSETLSLTCAVYGGSFSGYAW |
| | Variable Domain | SWIRQPPGKGLEWIGEIDHRGFTNYNPSLKSRVTIS |
| | | VDTSKNQFSLKLSSVTAADTAVYYCARVRYDSSD |
| | | SYYYSYDYGMDVWGQGTTVTVSS |
| 178 | VH (DNA) | CAGGTGCAGCTACAGCAGTGGGGCGCAGGACTG |
| | | TTGAAGCCTTCGGAGACCCTGTCCCTCACCTGCG |
| | | CTGTCTATGGTGGGTCCTTCTCCGGTTACGCATG |
| | | GAGCTGGATCCGCCAGCCCCCAGGGAAGGGGCT |
| | | GGAGTGGATTGGGGAAATCGACCATCGAGGATT |
| | | CACCAACTACAACCCGTCCCTCAAGAGTCGAGT |
| | | CACCATATCAGTAGACACGTCCAAGAACCAGTT |
| | | CTCCCTGAAGCTGAGCTCTGTGACCGCCGCGGA |
| | | CACGGCGGTGTACTACTGCGCCAGGGTCAGATA |
| | | CGACAGCAGCGACTCCTACTACTATAGCTACGA |
| | | TTATGGAATGGACGTATGGGGCCAGGGAACAAC |
| | | TGTCACCGTCTCCTCA |
| 179 | CDRH1 IMGT | GGSFSGYA |
| | (Prot) | |
| 180 | CDRH1 Kabat | GYAWS |
| | (Prot) | |
| 181 | CDRH1 Chothia | GGSFSG |
| | (Prot) | |
| 182 | CDRH2 IMGT | IDHRGFT |
| | (Prot) | |
| 183 | CDRH2 Kabat | EIDHRGFTNYNPSLKS |
| | (Prot) | |
| 184 | CDRH2 Chothia | DHRGF |
| | (Prot) | |
| 185 | CDRH3 IMGT | ARVRYDSSDSYYYSYDYGMDV |
| | (Prot) | |
| 186 | CDRH3 Kabat | VRYDSSDSYYYSYDYGMDV |
| | (Prot) | |
| 187 | CDRH3 Chothia | VRYDSSDSYYYSYDYGMDV |
| | (Prot) | |
| 188 | Light Chain | DIVLTQSPDSLAVSLGERATINCKSSQSVLYSSNNK |
| | Variable Domain | NYLAWYQQKPGQPPKLLIYWASSRESGVPDRFSG |
| | | SGSGTDFTLTISSLQAEDVAVYYCQQSYSFPWTFG |
| | | GGTKVEIK |
| 189 | VL (DNA) | GACATCGTGCTGACCCAGTCTCCAGACTCCCTGG |
| | | CTGTGTCTCTGGGCGAGAGGGCCACCATCAACT |
| | | GCAAGTCCAGCCAGAGTGTTTTATACAGCTCCA |
| | | ACAATAAGAACTACTTAGCTTGGTACCAGCAGA |
| | | AACCAGGACAGCCTCCTAAGCTGCTCATTTACTG |
| | | GGCATCTAGCCGGGAATCCGGGGTCCCTGACCG |
| | | ATTCAGTGGCAGCGGGTCTGGGACAGATTTCAC |
| | | TCTCACCATCAGCAGCCTGCAGGCTGAAGATGT |
| | | GGCAGTTTATTACTGTCAGCAGTCCTACTCCTTC |
| | | CCTTGGACTTTTGGCGGAGGGACCAAGGTTGAG |
| | | ATCAAA |
| 190 | CDRL1 IMGT | KSSQSVLYSSNNKNYLA |
| | (Prot) | |
| 191 | CDRL1 Kabat | KSSQSVLYSSNNKNYLA |
| | (Prot) | |
| 192 | CDRL1 Chothia | KSSQSVLYSSNNKNYLA |
| | (Prot) | |
| 193 | CDRL2 IMGT | WASSRES |
| | (Prot) | |
| 194 | CDRL2 Kabat | WASSRES |
| | (Prot) | |
| 195 | CDRL2 Chothia | WASSRES |
| | (Prot) | |
| 196 | CDRL3 IMGT | QQSYSFPWT |
| | (Prot) | |
| 197 | CDRL3 Kabat | QQSYSFPWT |
| | (Prot) | |
| 198 | CDRL3 Chothia | QQSYSFPWT |
| | (Prot) | |

TABLE 14
Exemplary Antibody Sequences 10 (Ab10)

| SEQ | | |
|---|---|---|
| ID | | |
| NO: | Description | Sequence |
| 199 | Heavy Chain | QVQLQQWGAGLLKPSETLSLTCAVYGGSFQKYY |
| | Variable | WSWIRQPPGKGLEWIGEIDTSGFTNYNPSLKSR |
| | Domain | VTISVDTSKNQFSLKLSSVTAADTAVYYCARVG |
| | | RYSYGYYITAFDIWGQGTTVTVSS |
| 200 | VH (DNA) | CAGGTGCAGCTACAGCAGTGGGGCGCAGGACTG |
| | | TTGAAGCCTTCGGAGACCCTGTCCCTCACCTGC |
| | | GCTGTCTATGGTGGGTCCTTCCAAAAATACTAC |
| | | TGGAGCTGGATCCGCCAGCCCCCAGGGAAGGGG |
| | | CTGGAGTGGATTGGGGAAATCGACACCAGTGGA |
| | | TTCACCAACTACAACCCGTCCCTCAAGAGTCGA |
| | | GTCACCATATCAGTAGACACGTCCAAGAACCAG |
| | | TTCTCCCTGAAGCTGAGCTCTGTGACCGCCGCG |
| | | GACACGGCGGTGTACTACTGCGCCAGAGTGGGA |
| | | AGGTACAGCTACGGATACTATATCACCGCATTC |
| | | GACATATGGGGTCAGGGTACAACTGTCACCGTC |
| | | TCCTCA |
| 201 | CDRH1 IMGT | GGSFQKYY |
| | (Prot) | |
| 202 | CDRH1 Kabat | KYYWS |
| | (Prot) | |
| 203 | CDRH1 | GGSFQK |
| | Chothia | |
| | (Prot) | |
| 204 | CDRH2 IMGT | IDTSGFT |
| | (Prot) | |
| 205 | CDRH2 Kabat | EIDTSGFTNYNPSLKS |
| | (Prot) | |
| 206 | CDRH2 | DTSGF |
| | Chothia | |
| | (Prot) | |
| 207 | CDRH3 IMGT | ARVGRYSYGYYITAFDI |
| | (Prot) | |
| 208 | CDRH3 Kabat | VGRYSYGYYITAFDI |
| | (Prot) | |
| 209 | CDRH3 | VGRYSYGYYITAFDI |
| | Chothia | |
| | (Prot) | |
| 210 | Light Chain | DIVMTQSPDSLAVSLGERATINCKSSQSVLYSSN |
| | Variable | NKNYLAWYQQKPGQPPKLLIYWASTRESGVPDRF |
| | Domain | SGSGSGTDFTLTISSLQAEDVAVYYCQQHYSFPF |
| | | TFGGGTKVEIK |
| 211 | VL (DNA) | GACATCGTGATGACCCAGTCTCCAGACTCCCTG |
| | | GCTGTGTCTCTGGGCGAGAGGGCCACCATCAAC |
| | | TGCAAGTCCAGCCAGAGTGTTTTATACAGCTCCA |
| | | ACAATAAGAACTACTTAGCTTGGTACCAGCAGA |
| | | AACCAGGACAGCCTCCTAAGCTGCTCATTTACTG |
| | | GGCATCTACCCGGGAATCCGGGGTCCCTGACCG |
| | | ATTCAGTGGCAGCGGGTCTGGGACAGATTTCAC |
| | | TCTCACCATCAGCAGCCTGCAGGCTGAAGATGT |
| | | GGCAGTTTATTACTGTCAGCAGCACTACTCCTTC |
| | | CCTTTCACTTTTGGCGGAGGGACCAAGGTTGAG |
| | | ATCAAA |
| 212 | CDRL1 IMGT | KSSQSVLYSSNNKNYLA |
| | (Prot) | |
| 213 | CDRL1 Kabat | KSSQSVLYSSNNKNYLA |
| | (Prot) | |
| 214 | CDRL1 | KSSQSVLYSSNNKNYLA |
| | Chothia | |
| | (Prot) | |
| 215 | CDRL2 IMGT | WASTRES |
| | (Prot) | |
| 216 | CDRL2 Kabat | WASTRES |
| | (Prot) | |
| 217 | CDRL2 | WASTRES |
| | Chothia | |
| | (Prot) | |
| 218 | CDRL3 IMGT | QQHYSFPFT |
| | (Prot) | |
| 219 | CDRL3 Kabat | QQHYSFPFT |
| | (Prot) | |
| 220 | CDRL3 | QQHYSFPFT |
| | Chothia | |
| | (Prot) | |

Exemplary anti-CD20 antibodies and fragments thereof suitable for use in the CARs, vectors, cells and methods disclosed herein can be found in International Patent Publication No. WO/2020/123691, published Jun. 18, 2020, which is specifically incorporated herein by reference in its entirety. In an embodiment an anti-CD20 CAR has an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 271.

(SEQ ID NO: 271)
QVQLVQSGAEVKKPGASVKVSCKASGYTFKEYGISWVRQAPGQGLEWMGW
ISAYSGHTYYAQKLQGRVTMTTDTSTSTAYMELRSLRSDDTAVYYCARGP
HYDDWSGFIIWFDPWGQGTLVTVSSGSTSGSGKPGSGEGSTKGDIQMTQS
PSSLSASVGDRVTITCRASQSISSYLNWYQQKPGKAPKLLIYAASSLQSG
VPSRFSGSGSGTDFTLTISSLQPEDFATYYCQQSYRFPPTFGQGTKVEIK
AAAFVPVFLPAKPTTTPAPRPPTPAPTIASQPLSLRPEACRPAAGGAVHT
RGLDFACDIYIWAPLAGTCGVLLLSLVITLYCNHRNRFSVVKRGRKKLLY
IFKQPFMRPVQTTQEEDGCSCRFPEEEEGGCELRVKFSRSADAPAYQQGQ
NQLYNELNLGRREEYDVLDKRRGRDPEMGGKPRRKNPQEGLYNELQKDKM
AEAYSEIGMKGERRRGKGHDGLYQGLSTATKDTYDALHMQALPPR.

The present disclosure comprises antigen binding systems, such as iNKT cells, that comprise a CAR that includes anti-CD20 binding motif and a CAR that includes anti-CD19 binding motif. The present specification comprises a variety of other target antigens for the first and/or second CAR, comprising, without limitation, a first or second antigen that is 5T4, alphafetoprotein, B cell maturation antigen (BCMA), TACI, CA-125, carcinoembryonic antigen, CD19, CD20, CD22, CD23, CD30, CD33, CD56, CD123, CD138, c-Met, CSPG4, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gp120, IL-11Ralpha, kappa chain, lambda chain, melanoma-associated antigen, mesothelin, MUC-1, mutated p53, mutated ras, prostate-specific antigen, ROR1, VEGFR2, or a combination thereof. Accordingly in various embodiments, an antigen-binding system of the present disclosure may comprise a first binding motif that binds a first antigen and a second binding motif that binds a second different antigen each of which is selected from 5T4, alphafetoprotein, B cell maturation antigen (BCMA), CA-125, carcinoembryonic antigen, CD19, CD20, CD22, CD23, CD30, CD33, CD56, CD123, CD138, c-Met, CSPG4, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gp120, IL-11Ralpha, kappa chain, lambda chain, melanoma-associated antigen, mesothelin, MUC-1, mutated p53, mutated ras, prostate-specific antigen, ROR1, VEGFR2, EphA3 (EPH receptor A3), BAFFR (B-cell activating factor receptor), or a combination thereof.

CD19 (also known as Cluster of Differentiation 19, B-lymphocyte antigen CD19, B-lymphocyte surface antigen B4, B4, CVID3, Differentiation antigen CD19) is a protein that is encoded by the CD19 gene in humans. Unless otherwise indicated, it is to be appreciated the references to CD19 in the present disclosure relate to human CD19. It is found on the surface of B cells. Since CD19 expression is a hallmark of B cells, it may be useful as an antigen, e.g., in recognizing B cells and cancer cells that arise from B cells, e.g., B-cell lymphomas. Anti-CD19 antibodies may bind CD19 expressed on, e.g., B lymphocytes in peripheral blood and spleen, B cell chronic lymphocytic leukemia (B-CLL) cells, pro lymphocytic leukemia (PLL) cells, hairy cell leukemia (HCL) cells, common acute lymphoblastic leukemia (CALL) cells, pre-B acute lymphoblastic leukemia (pre-B-ALL) cells, and NULL-acute lymphoblastic leukemia (NULL-ALL) cells, to provide a few non limiting examples. An exemplary pharmaceutical product that comprises an antigen binding system that comprises an anti-CD19 binding motif is the pharmaceutical product YESCARTA®. YESCARTA® is a CD19-directed genetically modified autologous T cell immunotherapy indicated for the treatment of adult patients with relapsed or refractory large B-cell lymphoma after two or more lines of systemic therapy, comprising diffuse large B-cell lymphoma (DLBCL) not otherwise specified, primary mediastinal large B-cell lymphoma, high grade B-cell lymphoma, and DLBCL arising from follicular lymphoma (See YESCARTA® FDA-approved package insert, the entirety of which is incorporated herein by reference with respect to methods and compositions relating to immunotherapy). Another exemplary pharmaceutical product that comprises an antigen binding system that comprises an anti-CD19 binding motif is the pharmaceutical product KYMRIAH®. KYMRIAH® is a CD19-directed genetically modified autologous T-cell immunotherapy indicated for the treatment of: (1) Patients up to 25 years of age with B-cell precursor acute lymphoblastic leukemia (ALL) that is refractory or in second or later relapse; and (2) Adult patients with relapsed or refractory (r/r) large B-cell lymphoma after two or more lines of systemic therapy comprising diffuse large B-cell lymphoma (DLBCL) not otherwise specified, high grade B-cell lymphoma and DLBCL arising from follicular lymphoma (See KYMRIAH® FDA-approved package insert, the entirety of which is incorporated herein by reference with respect to methods and compositions relating to immunotherapy).

Both YESCARTA® and KYMRIAH® comprise antibody binding domains derived from an anti-human CD19 antibody. Many anti-CD19 antibodies are thought to bind an epitope of CD19 encoded in exon 4 of the CD19 gene. Other anti-CD19 binding motifs may recognize different epitopes of CD19, or the same epitope with differential affinities. Antigen binding systems may comprise antigen binding domains derived, for example, from SJ25C1. The CD19 antibody, clone SJ25C1 was derived from hybridization of Sp2/0 mouse myeloma cells with spleen cells isolated from BALB/c mice immunized with NALM1 and NALM16 cells. SJ25C1 antigen binding domains were used in other investigational CD19-targeting chimeric antigen receptor (CAR) T-cell therapy.

An anti-CD19 binding CAR of the present disclosure may comprise antigen-binding sequences as found in an antibody described herein. In various embodiments, an anti-CD19 binding motif of the present disclosure comprises at least one HCDR provided herein, e.g., at least one HCDR disclosed in Table 15. In various embodiments, an anti-CD19 binding motif of the present disclosure comprises two HCDRs provided herein, e.g., at least two HCDRs disclosed in Table 15. In various embodiments, an anti-CD19 binding motif of the present disclosure comprises three HCDRs provided herein, e.g., three HCDRs disclosed in Table 15.

In various embodiments, an anti-CD19 binding motif of the present disclosure comprises at least one LCDR provided herein, e.g., at least one LCDR disclosed in Table 15. In various embodiments, an anti-CD19 binding motif of the present disclosure comprises two LCDRs provided herein, e.g., at least two LCDRs disclosed in Table 15. In various embodiments, an anti-CD19 binding motif of the present disclosure comprises three LCDRs provided herein, e.g., three LCDRs disclosed in Table 15.

In various embodiments, an anti-CD19 binding motif of the present disclosure comprises at least one HCDR provided herein, e.g., at least one HCDR disclosed in Table 15, and at least one LCDR provided herein, e.g., at least one LCDR disclosed in Table 15. In various embodiments, an anti-CD19 binding motif of the present disclosure comprises two HCDRs provided herein, e.g., at least two HCDRs disclosed in Table 15, and two LCDRs provided herein, e.g., at least two LCDRs disclosed in Table 15. In various embodiments, an anti-CD19 binding motif of the present disclosure comprises three HCDRs provided herein, e.g., three HCDRs disclosed in Table 15, and three LCDRs provided herein, e.g., three LCDRs disclosed in Table 15.

In various embodiments, an anti-CD19 binding motif of the present disclosure comprises at least one heavy chain framework region (heavy chain FR) of a heavy chain variable domain disclosed herein, e.g., at least one heavy chain FR of a heavy chain variable domain disclosed in Table 15. In various embodiments, an anti-CD19 binding motif of the present disclosure comprises two heavy chain FRs of a heavy chain variable domain disclosed herein, e.g., at least two heavy chain FRs of a heavy chain variable domain disclosed in Table 15. In various embodiments, an anti-CD19 binding motif of the present disclosure comprises three heavy chain FRs of a heavy chain variable domain disclosed herein, e.g., three heavy chain FRs of a heavy chain variable domain disclosed in Table 15.

In various embodiments, an anti-CD19 binding motif of the present disclosure comprises at least one light chain FR of a light chain variable domain disclosed herein, e.g., at least one light chain FR of a light chain variable domain disclosed in Table 15. In various embodiments, an anti-CD19 binding motif of the present disclosure comprises two light chain FRs of a light chain variable domain disclosed herein, e.g., at least two light chain FRs of a light chain variable domain disclosed in Table 15. In various embodiments, an anti-CD19 binding motif of the present disclosure comprises three light chain FRs of a light chain variable domain disclosed herein, e.g., three light chain FRs of a light chain variable domain disclosed in Table 15.

In various embodiments, an anti-CD19 binding motif of the present disclosure comprises at least one heavy chain FR of a heavy chain variable domain disclosed herein, e.g., at least one heavy chain FR of a heavy chain variable domain disclosed in Table 15, and at least one light chain FR of a light chain variable domain disclosed herein, e.g., at least one light chain FR of a light chain variable domain disclosed in Table 15. In various embodiments, an anti-CD19 binding motif of the present disclosure comprises two heavy chain FRs of a heavy chain variable domain disclosed herein, e.g., at least two heavy chain FRs of a heavy chain variable domain disclosed in Table 15, and two light chain FRs of a light chain variable domain disclosed herein, e.g., at least two light chain FRs of a light chain variable domain disclosed in Table 15. In various embodiments, an anti-CD19 binding motif of the present disclosure comprises three heavy chain FRs of a heavy chain variable domain disclosed herein, e.g., three heavy chain FRs of a heavy chain variable domain disclosed in Table 15, and three light chain FRs of a light chain variable domain disclosed herein, e.g., three light chain FRs of a light chain variable domain disclosed in Table 15.

In various embodiments, an anti-CD19 binding motif of the present disclosure comprises one, two, or three FRs that together or each individually have at least 75% identity (e.g., at least 75%, at least 80%, at least 90%, at least 95%, or 100% identity; e.g., 85-90%, 85-95%, 85-100%, 90-95%, 90-100%, or 95-100%) to corresponding FR(s) of a heavy chain variable domain of a heavy chain variable domain disclosed in in Table 15. In various embodiments, an anti-CD19 binding motif of the present disclosure comprises one, two, or three FRs that together or each individually have at least 75% identity (e.g., at least 75%, at least 80%, at least 90%, at least 95%, or 100% identity; e.g., 85-90%, 85-95%, 85-100%, 90-95%, 90-100%, or 95-100%) to corresponding FR(s) of a light chain variable domain of a light chain variable domain disclosed in Table 15.

In various embodiments, an anti-CD19 binding motif of the present disclosure comprises at least one heavy chain variable domain having at least 75% sequence identity to a heavy chain variable domain disclosed in Table 15 (e.g., at least 75%, at least 80%, at least 90%, at least 95%, or 100% identity; e.g., 85-90%, 85-95%, 85-100%, 90-95%, 90-100%, or 95-100%). In various embodiments, an anti-CD19 binding motif of the present disclosure comprises two heavy chain variable domains each having at least 75% sequence identity to a heavy chain variable domain disclosed in Table 15 (e.g., at least 75%, at least 80%, at least 90%, at least 95%, or 100% identity; e.g., 85-90%, 85-95%, 85-100%, 90-95%, 90-100%, or 95-100%), which heavy chain variable domains may be same or different.

In various embodiments, an anti-CD19 binding motif of the present disclosure comprises at least one light chain variable domain having at least 75% sequence identity to a light chain variable domain disclosed in Table 15 (e.g., at least 75%, at least 80%, at least 90%, at least 95%, or 100% identity; e.g., 85-90%, 85-95%, 85-100%, 90-95%, 90-100%, or 95-100%). In various embodiments, an anti-CD19 binding motif of the present disclosure comprises two light chain variable domains each having at least 75% sequence identity to a light chain variable domain disclosed in Table 15 (e.g., at least 75%, at least 80%, at least 90%, at least 95%, or 100% identity; e.g., 85-90%, 85-95%, 85-100%, 90-95%, 90-100%, or 95-100%), which light chain variable domains may be same or different.

In various embodiments, an anti-CD19 binding motif of the present disclosure comprises at least one heavy chain variable domain having at least 75% sequence identity to a heavy chain variable domain disclosed in Table 15 (e.g., at least 75%, at least 80%, at least 90%, at least 95%, or 100% identity; e.g., 85-90%, 85-95%, 85-100%, 90-95%, 90-100%, or 95-100%) and at least one light chain variable domain having at least 75% sequence identity to a light chain variable domain disclosed in Table 15 (e.g., at least 75%, at least 80%, at least 90%, at least 95%, or 100% identity; e.g., 85-90%, 85-95%, 85-100%, 90-95%, 90-100%, or 95-100%).

In various embodiments, an anti-CD19 binding motif of the present disclosure comprises two heavy chain variable domains each having at least 75% sequence identity to a heavy chain variable domain disclosed in Table 15 (e.g., at least 75%, at least 80%, at least 90%, at least 95%, or 100% identity; e.g., 85-90%, 85-95%, 85-100%, 90-95%, 90-100%, or 95-100%) and two light chain variable domains each having at least 75% sequence identity to a light chain variable domain disclosed in Table 15 (e.g., at least 75%, at least 80%, at least 90%, at least 95%, or 100% identity; e.g., 85-90%, 85-95%, 85-100%, 90-95%, 90-100%, or 95-100%), where, in various embodiments, (i) each of the heavy chain variable domains may be same or different; or (ii) each of the light chain variable domains may be same or different.

In certain embodiments, an anti-CD19 binding motif of the present disclosure comprises a binding motif that comprises a heavy chain variable domain of the present disclosure, a light chain variable domain of the present disclosure, and a linker having at least 75% sequence identity to SEQ ID NO: 247 (e.g., at least 75%, at least 80%, at least 90%, at least 95%, or 100% identity; e.g., 85-90%, 85-95%, 85-100%, 90-95%, 90-100%, or 95-100%). In certain embodiments, an anti-CD19 binding motif of the present disclosure comprises a binding motif that comprises a linker according to SEQ ID NO: 247 and/or a linker according to any one of SEQ ID NOs: 293, 294, and 296-300. In certain embodiments, an anti-CD19 binding motif of the present disclosure comprises a binding motif that comprises a heavy chain variable domain of the present disclosure, a light chain variable domain of the present disclosure, and a leader sequence having at least 75% sequence identity to SEQ ID NO: 245 (e.g., at least 75%, at least 80%, at least 90%, at least 95%, or 100% identity; e.g., 85-90%, 85-95%, 85-100%, 90-95%, 90-100%, or 95-100%). In certain embodiments, an anti-CD19 binding motif of the present disclosure comprises a binding motif that comprises a CSF2RA leader sequence according to SEQ ID NO: 245. In certain embodiments, an anti-CD19 binding motif of the present disclosure comprises a binding motif that comprises a heavy chain variable domain of the present disclosure, a light chain variable domain of the present disclosure, a linker of the present disclosure, and a leader sequence of the present disclosure. In certain embodiments a binding motif has the sequence set forth in SEQ ID NO: 243. Exemplary nucleotide sequences encoding an anti-CD19 binding motif and components thereof are found in SEQ ID NOs: 244, 246, and 248. In various embodiments a binding motif may comprise a linker according to SEQ ID NO: 247 and/or a linker according to any one of SEQ ID NOs: 293, 294, and 296-300 adjacent to one or more additional linkers.

TABLE 15
Exemplary anti-CD19 Antibody Sequences (Ab11)

| SEQ | | |
|---|---|---|
| ID | | |
| NO: | Description | Sequence |
| 221 | Heavy Chain | EVKLOESGPGLVAPSOSLSVTCTVSGVSLPDYGVSWIR |
| | Variable | OPPRKGLEWLGVIWGSETTYYNSALKSRLTIIKDNSKS |
| | Domain | OVFLKMNSLOTDDTAIYYCAKHYYYGGSYAMDYWG |
| | | QGTSVTVSS |
| 222 | VH (DNA) | GAGGTGAAACTGCAGGAGTCAGGACCTGGCCTGGT |
| | | GGCGCCCTCACAGAGCCTGTCCGTCACATGCACTGT |
| | | CTCAGGGGTCTCATTACCCGACTATGGTGTAAGCTG |
| | | GATTCGCCAGCCTCCACGAAAGGGTCTGGAGTGGCT |
| | | GGGAGTAATATGGGGTAGTGAAACCACATACTATA |
| | | ATTCAGCTCTCAAATCCAGACTGACCATCATCAAGG |
| | | ACAACTCCAAGAGCCAAGTTTTCTTAAAAATGAACA |
| | | GTCTGCAAACTGATGACACAGCCATTTACTACTGTG |
| | | CCAAACATTATTACTACGGTGGTAGCTATGCTATGG |
| | | ACTACTGGGGTCAAGGAACCTCAGTCACCGTCTCCT |
| | | CA |
| 223 | CDRH1 IMGT | GVSLPDYG |
| | (Prot) | |
| 224 | CDRH1 Kabat | DYGVS |
| | (Prot) | |
| 225 | CDRH1 Chothia | GVSLPDY |
| | (Prot) | |
| 226 | CDRH2 IMGT | IWGSETT |
| | (Prot) | |
| 227 | CDRH2 Kabat | VIWGSETTYYNSALKS |
| | (Prot) | |
| 228 | CDRH2 Chothia | WGSET |
| | (Prot) | |
| 229 | CDRH3 IMGT | AKHYYYGGSYAMDY |
| | (Prot) | |
| 230 | CDRH3 Kabat | HYYYGGSYAMDY |
| | (Prot) | |
| 231 | CDRH3 Chothia | HYYYGGSYAMDY |
| | (Prot) | |
| 232 | Light Chain | DIOMTQTTSSLSASLGDRVTISCRASQDISKYLNWYOO |
| | Variable | KPDGTVKLLIYHTSRLHSGVPSRFSGSGSGTDYSLTISN |
| | Domain | LEOEDIATYFCOOGNTLPYTFGGGTKLEIT |
| 233 | VL (DNA) | GACATCCAGATGACACAGACTACATCCTCCCTGTCT |
| | | GCCTCTCTGGGAGACAGAGTCACCATCAGTTGCAGG |
| | | GCAAGTCAGGACATTAGTAAATATTTAAATTGGTAT |
| | | CAGCAGAAACCAGATGGAACTGTTAAACTCCTGAT |
| | | CTACCATACATCAAGATTACACTCAGGAGTCCCATC |
| | | AAGGTTCAGTGGCAGTGGGTCTGGAACAGATTATTC |
| | | TCTCACCATTAGCAACCTGGAGCAAGAAGATATTGC |
| | | CACTTACTTTTGCCAACAGGGTAATACGCTTCCGTA |
| | | CACGTTCGGAGGGGGGACTAAGTTGGAAATAACA |
| 234 | CDRL1 IMGT | RASQDISKYLN |
| | (Prot) | |
| 235 | CDRL1 Kabat | RASQDISKYLN |
| | (Prot) | |
| 236 | CDRL1 Chothia | RASQDISKYLN |
| | (Prot) | |
| 237 | CDRL2 IMGT | HTSRLHS |
| | (Prot) | |
| 238 | CDRL2 Kabat | HTSRLHS |
| | (Prot) | |
| 239 | CDRL2 Chothia | HTSRLHS |
| | (Prot) | |
| 240 | CDRL3 IMGT | QQGNTLPYT |
| | (Prot) | |
| 241 | CDRL3 Kabat | QQGNTLPYT |
| | (Prot) | |
| 242 | CDRL3 Chothia | QQGNTLPYT |
| | (Prot) | |
| 243 | binding motif | DIQMTQTTSSLSASLGDRVTISCRASQDISKYLNWYQQ |
| | (Prot) | KPDGTVKLLIYHTSRLHSGVPSRFSGSGSGTDYSLTISN |
| | | LEQEDIATYFCQQGNTLPYTFGGGTKLEITGSTSGSGK |
| | | PGSGEGSTKGEVKLQESGPGLVAPSQSLSVTCTVSGVS |
| | | LPDYGVSWIRQPPRKGLEWLGVIWGSETTYYNSALKS |
| | | RLTIIKDNSKSQVFLKMNSLQTDDTAIYYCAKHYYYG |
| | | GSYAMDYWGQGTSVTVSS |
| 244 | binding motif | gacatccagatgacacagactacatcctccctgtctgcctctctgggagacagagtca |
| | (DNA) | ccatcagttgcagggcaagtcaggacattagtaaatatttaaattggtatcagcagaaa |
| | | ccagatggaactgttaaactcctgatctaccatacatcaagattacactcaggagtccc |
| | | atcaaggttcagtggcagtgggtctggaacagattattctctcaccattagcaacctgg |
| | | agcaagaagatattgccacttacttttgccaacagggtaatacgcttccgtacacgttcg |
| | | gaggggggactaagttggaaataacaggctccacctctggatccggcaagcccgga |
| | | tctggcgagggatccaccaagggcgaggtgaaactgcaggagtcaggacctggcc |
| | | tggtggcgccctcacagagcctgtccgtcacatgcactgtctcaggggtctcattacc |
| | | cgactatggtgtaagctggattcgccagcctccacgaaagggtctggagtggctggg |
| | | agtaatatggggtagtgaaaccacatactataattcagctctcaaatccagactgacca |
| | | tcatcaaggacaactccaagagccaagttttcttaaaaatgaacagtctgcaaactgat |
| | | gacacagccatttactactgtgccaaacattattactacggtggtagctatgctatggac |
| | | tactggggtcaaggaacctcagtcaccgtctcctca |
| 245 | Leader (CSF2RA) | MLLLVTSLLLCELPHPAFLLIP |
| | (Prot) | |
| 246 | Leader (CSF2RA) | atgcttctcctggtgacaagccttctgctctgtgagttaccacacccagcattcctcctg |
| | (DNA) | atccca |
| 247 | Linker | GSTSGSGKPGSGEGSTKG |
| | (Prot) | |
| 248 | Linker | ggctccacctctggatccggcaagcccggatctggcgagggatccaccaagggc |
| | (DNA) | |

In an embodiment an anti-CD19 CAR has an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 272.

(SEQ ID NO: 272)
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGSTSGSGKPGSGEGSTKGEVKLQESGPGLVAPSQSLSVTCTVS
GVSLPDYGVSWIRQPPRKGLEWLGVIWGSETTYYNSALKSRLTIIKDNSK
SQVFLKMNSLQTDDTAIYYCAKHYYYGGSYAMDYWGQGTSVTVSSAAAIE
VMYPPPYLDNEKSNGTIIHVKGKHLCPSPLFPGPSKPFWVLVVVGGVLAC
YSLLVTVAFIIFWVRSKRSRLLHSDYMNMTPRRPGPTRKHYQPYAPPRDF
AAYRSRVKFSRSADAPAYQQGQNQLYNELNLGRREEYDVLDKRRGRDPEM
GGKPRRKNPQEGLYNELQKDKMAEAYSEIGMKGERRRGKGHDGLYQGLST
ATKDTYDALHMQALPPR.

In various embodiments, the first CAR and a second CAR of the duel targeting system. differ only with respect to the binding motif. In various embodiments, a bicistronic CAR comprises a first CAR and a second CAR where the sequence of the first CAR and the second CAR differ only with respect to a heavy chain variable domain sequence and/or a light chain variable domain sequence. Thus, in some embodiments, a first CAR and a second CAR of a duel targeting may have same or different sequences for any or all of one or more components thereof, e.g., same or different costimulatory domains. For example, one or both of a first CAR and a second CAR of the duel targeting may comprise a costimulatory domain provided herein, such as a CD28, 41BB, OX40, or ICOS costimulatory domain.

A CAR of a duel targeting may comprise a binding motif, a hinge, a transmembrane domain, and an intracellular domain comprising a costimulatory domain and an activation domain. The binding motif may be an anti-CD19 or an anti-CD20 binding motif of the present disclosure. A hinge and transmembrane domain may be a 28T (CD28) domain or a CD8 domain that comprises a hinge domain and a transmembrane domain. A costimulatory domain may be a CD28 or 41BB costimulatory domain. An activation domain may be a CD3z activation domain.

Chimeric antigen receptors (CARs) are engineered receptors that may direct or redirect cells, such as iNKT cells to target a selected antigen. A CAR may be engineered to recognize an antigen and, when bound to that antigen, activate the immune cell to attack and destroy the cell bearing that antigen. When these antigens exist on tumor cells, an immune cell that expresses the CAR may target and kill the tumor cell. CARs generally comprise an extracellular binding motif that mediates antigen binding (e.g., an anti-CD20 and/or an anti-CD19 binding motif), a transmembrane domain that spans, or is understood to span, the cell membrane when the antigen binding system is present at a cell surface or cell membrane, and an intracellular (or cytoplasmic) signaling domain.

According to at least one non-limiting view, there have been at least three “generations” of CAR compositions. In a first generation of CARs, a binding motif (e.g., a single chain fragment variable, binding motif) is linked or connected to a signaling domain (e.g., CD3ζ) via a transmembrane domain, optionally comprising a hinge domain and one or more spacers. In a second generation of CARs, a costimulatory domain (CM1, such as CD28, 4-1BB, or OX-40) is introduced with the signaling domain (e.g., CD3ζ). In a third generation of CARs, a second costimulatory domain (CM2) is comprised.

TCRs are heterodimers composed of an α-chain and a β-chain. TCR signaling requires recruitment of signaling proteins that generate an immune synapse. In addition, TCR localization at the plasma membrane depends on CD3 complex, which is expressed in T cells.

One or more antigen binding motifs determine the target(s) of an antigen binding system. A binding motif of an antigen binding system may comprise any binding motif, e.g., an antibody provided by the present disclosure, e.g., a binding motif of the present disclosure. In some embodiments, a binding motif may comprise an anti-CD20 binding motif or anti-CD19 binding motif.

Binding motifs are used in chimeric antigen receptors at least in part because they may be engineered to be expressed as part of a single chain along with the other CAR components. See, for example, U.S. Pat. Nos. 7,741,465, and 6,319,494 as well as Eshhar et al., Cancer Immunol Immunotherapy (1997) 45: 131-136, Krause et al., J. Exp. Med., Volume 188, No. 4, 1998 (619-626); Finney et al., Journal of Immunology, 1998, 161: 2791-2797, each of which is incorporated herein by reference with respect to binding motif domains in CARs. A binding motif, or scFv, is a single chain antigen binding fragment comprising a heavy chain variable domain and a light chain variable domain, which heavy chain variable domain and light chain variable domain are linked or connected together. See, for example, U.S. Pat. Nos. 7,741,465, and 6,319,494 as well as Eshhar et al., Cancer Immunol Immunotherapy (1997) 45: 131-136, each of which is incorporated herein by reference with respect to binding motif domains. When derived from a parent antibody, a binding motif may retain some of, retain all of, or essentially retain the parent antibody's binding of a target antigen.

In certain embodiments, the CARs contemplated herein may comprise linker residues between the various domains, e.g., between VH and VL domains, added for appropriate spacing conformation of the molecule. CARs contemplated herein, may comprise one, two, three, four, or five or more linkers. In some embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

Illustrative examples of linkers include glycine polymers (G)n; glycine-serine polymers (G₁₋₅S₁₋₅)n, where n is an integer of at least one, two, three, four, or five; glycine-alanine polymers; alanine-serine polymers; and other flexible linkers known in the art. In some embodiments, the linker may comprise a linker according to SEQ ID NO: 302. Glycine and glycine-serine polymers are relatively unstructured, and therefore may be able to serve as a neutral tether between domains of fusion proteins such as the CARs described herein. Glycine accesses more phi-psi space than even alanine, and is much less restricted than residues with longer side chains (see Scheraga, Rev. Computational Chem. 11173-142 (1992)). Other linkers contemplated herein include Whitlow linkers (see Whitlow, Protein Eng. 6(8): 989-95 (1993)). The ordinarily skilled artisan will recognize that design of a CAR in some embodiments may include linkers that are all or partially flexible, such that the linker may include a flexible linker as well as one or more portions that confer less flexible structure to provide for a desired CAR structure.

In embodiments, a CAR comprises a scFv that further comprises a variable region linking sequence. A “variable region linking sequence,” is an amino acid sequence that connects a heavy chain variable region to a light chain variable region and provides a spacer function compatible with interaction of the two sub-binding domains so that the resulting polypeptide retains a specific binding affinity to the same target molecule as an antibody that comprises the same light and heavy chain variable regions. In one embodiment, the variable region linking sequence is 1, 2, 3, 4, 5, 6, 7, 8, 9, 10, 11, 12, 13, 14, 15, 16, 17, 18, 19, 20, 21, 22, 23, 24, 25, or more amino acids long.

TABLE 16
Exemplary linkers

| | SEQ | |
|---|---|---|
| | ID NO: | Sequence |
| G4S (G4Sx1) | 293 | GGGGS |
| G4Sx2 | 294 | GGGGSGGGGS |
| G4Sx3 | 296 | GGGGSGGGGSGGGGS |
| G4Sx4 | 297 | GGGGSGGGGSGGGGSGGGGS |
| IgA | 298 | PSTPPTPSPSTPPTPSPS |
| PAPAP | 299 | PAPAP (optionally comprising 1, |
| | | 2, 3, 4, 5, 6, 7, 8, 9, or 10 |
| | | adjacent copies of PAPAP) |
| EAAAK | 300 | EAAAK (optionally comprising 1, |
| | | 2, 3, 4, 5, 6, 7, 8, 9, or 10 |
| | | adjacent copies of EAAAK) |

In embodiments, the binding domain of the CAR is followed by one or more “spacer domains,” which refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region.

The binding domain of the CAR may generally be followed by one or more “hinge domains,” which plays a role in positioning the antigen binding domain away from the effector cell surface to enable proper cell/cell contact, antigen binding and activation. A CAR generally comprises one or more hinge domains between the binding domain and the transmembrane domain. The hinge domain may be derived either from a natural, synthetic, semi-synthetic, or recombinant source. The hinge domain may include the amino acid sequence of a naturally occurring immunoglobulin hinge region or an altered immunoglobulin hinge region.

In some embodiments, an antigen binding system of the present disclosure may comprise a hinge that is, is from, or is derived from (e.g., comprises all or a fragment of) an immunoglobulin-like hinge domain. In some embodiments, a hinge domain is from or derived from an immunoglobulin. In some embodiments, a hinge domain is selected from the hinge of IgG1, IgG2, IgG3, IgG4, IgA, IgD, IgE, or IgM, or a fragment thereof. A hinge may be derived from a natural source or from a synthetic source. Hinge domains suitable for use in the CARs described herein include the hinge region derived from the extracellular regions of type 1 membrane proteins such as CD8a, CD4, CD28 and CD7, which may be wild-type hinge regions from these molecules or may be altered, for example a truncated CD28 hinge domain. A hinge may be derived from a natural source or from a synthetic source. In some embodiments, an Antigen binding system of the present disclosure may comprise a hinge that is, is from, or is derived from (e.g., comprises all or a fragment of) CD2, CD3 delta, CD3 epsilon, CD3 gamma, CD4, CD7, CD8a, CD80, CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA1-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, or which is a fragment or combination thereof.

In some embodiments, an antigen binding system of the present disclosure may comprise a hinge that is, is from, or is derived from (e.g., comprises all or a fragment of) a hinge of CD8 alpha. In some embodiments a hinge is, is from, or is derived from a hinge of CD28. In some embodiments, a hinge is, is from, or is derived from a fragment of a hinge of CD8 alpha or a fragment of a hinge of CD28, wherein the fragment is anything less than the whole. In some embodiments, a fragment of a CD8 alpha hinge or a fragment of a CD28 hinge comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of a CD8 alpha hinge, or of a CD28 hinge. Exemplary hinge sequences may comprise those provided in Table 17 (SEQ ID NOs: 261-269).

TABLE 17
Exemplary hinges

| SEQ | | |
|---|---|---|
| ID NO | Hinge | Sequence |
| 261 | C8K | GCAGCTGCTTTCGTGCCTGTGTTCCTGCCTGCTAAGCCCACCACCACTC |
| | | CTGCTCCAAGACCTCCTACCCCCGCTCCTACAATCGCCAGCCAACCTCT |
| | | GAGCCTGAGACCGGAGGCATGCAGACCTGCGGCAGGGGGAGCAGTTC |
| | | ACACAAGAGGCTTGGACTTCGCTTGCGACATCTACATCTGGGCCCCTCT |
| | | GGCCGGCACATGCGGAGTTCTTCTTCTTAGCCTGGTGATCACCCTGTAC |
| | | TGCAACCACAGAAAC |
| 262 | C28T | GCTGCTGCATTGGATAATGAAAAATCGAACGGCACAATCATTCATGTG |
| | | AAGGGCAAACACCTGTGTCCCAGCCCCTTGTTCCCAGGACCTAGCAAG |
| | | CCTTTTTGGGTTCTCGTGGTGGTGGGCGGCGTTCTGGCTTGCTACTCTCT |
| | | ACTTGTAACTGTCGCATTTATTATATTCTGGGTT |
| 263 | C28T1x | GGAGGAGGAGGATCTCTGGATAACGAGAAAAGCAACGGGACCATCAT |
| | | TCATGTGAAGGGAAAACATCTGTGTCCCAGCCCCTTGTTCCCCGGACCT |
| | | AGCAAGCCGTTTTGGGTTCTCGTGGTGGTGGGCGGCGTTCTGGCTTGCT |
| | | ACTCTCTGCTTGTGACCGTTGCCTTCATTATCTTCTGGGTT |
| 264 | C28T2x | GGAGGAGGAGGATCTGGTGGAGGAGGTTCTCTGGACAATGAGAAATC |
| | | AAATGGAACGATCATCCATGTGAAGGGGAAGCACCTCTGCCCCTCTCC |
| | | CCTGTTTCCTGGTCCTAGCAAGCCCTTCTGGGTTTTGGTGGTCGTGGGC |
| | | GGCGTTCTGGCTTGCTACAGCCTGTTAGTGACCGTTGCATTTATCATAT |
| | | TTTGGGTT |
| 265 | C28T2x_ | GGAGGAGGAGGATCTGGTGGAGGAGGTTCTCTGGACAATGAGAAATC |
| | NC | GAATGGGACAATCATCCATGTGAAGGGGAAGCACCTGAGCCCCTCTCC |
| | | CCTGTTTCCTGGTCCTAGCAAGCCCTTCTGGGTTTTGGTGGTCGTGGGC |
| | | GGCGTTCTGGCCGTTTACAGCCTGTTAGTGACCGTTGCTTTTATCATATT |
| | | TTGGGTT |
| 266 | C28T2x_ | GGAGGAGGAGGATCTGGTGGAGGAGGTTCTCTGGACAATGAAAAGAG |
| | NG | CAATGGCACAATCATCCATGTGAAGGGGAAGCACCTGAACGGCTCCGC |
| | | CCTGTTTCCTGGTCCTAGCAAGCCATTTTGGGTTCTCGTGGTGGTGGGC |
| | | GGCGTTCTGGCCGTTTACAGCCTGTTAGTGACCGTTGCGTTCATAATCT |
| | | TCTGGGTT |
| 267 | C28T3x | GGAGGAGGAGGATCTGGTGGAGGAGGTTCTGGAGGAGGCGGCTCTCTC |
| | | GACAACGAAAAGAGCAATGGCACCATTATTCACGTTAAAGGCAAGCAT |
| | | CTGTGCCCCTCCCCCCTGTTCCCCGGACCTTCAAAACCTTTTTGGGTTCT |
| | | CGTGGTGGTGGGCGGCGTTCTGGCCTGCTATTCTTTGCTGGTAACTGTA |
| | | GCCTTCATTATCTTCTGGGTT |
| 268 | 14 | GAGAGCAAGTACGGACCTCCTTGTCCTCCATGTCCTGCTCCCGAGTTCG |
| | | AGGGCGGACCTTCAGTGTTCCTGTTCCCCCCTAAACCCAAGGATACTCT |
| | | TATGATCAGCCGGACCCCCGAGGTCACCTGTGTGGTGGTAGATGTTAG |
| | | CCAGGAGGATCCCGAGGTGCAGTTCAACTGGTACGTCGACGGCGTCGA |
| | | GGTACACAACGCCAAGACCAAGCCTAGGGAGGAGCAGTTCCAGTCCAC |
| | | CTATAGGGTCGTGAGCGTGCTTACCGTGCTGCACCAGGACTGGTTGAA |
| | | CGGCAAGGAGTACAAGTGCAAGGTGTCCAACAAGGGCCTCCCCAGCA |
| | | GCATCGAGAAGACCATTAGCAAGGCAAAGGGACAGCCCAGGGAGCCC |
| | | CAGGTGTACACATTACCTCCTTCCCAGGAAGAGATGACCAAGAACCAG |
| | | GTGTCGCTTACCTGCCTGGTCAAGGGCTTCTACCCCTCCGACATTGCAG |
| | | TTGAATGGGAGTCAAACGGCCAGCCGGAGAACAATTACAAGACCACCC |
| | | CCCCAGTCTTGGACAGCGACGGCTCTTTCTTCCTCTACTCGCGGCTTAC |
| | | TGTAGATAAAAGTCGTTGGCAGGAGGGAAACGTGTTCAGCTGCTCTGT |
| | | GATGCACGAGGCCCTCCATAACCACTACACCCAGAAGAGCCTCTCCCT |
| | | GTCTCTGGGCAAGATGTTCTGGGTGCTGGTCGTGGTGGGCGGAGTTCTT |
| | | GCTTGCTACTCCCTGCTCGTGACCGTCGCTTTCATTATATTCTGGGTC |
| 269 | 11-2 | GAGAGAAAGTGTTGTGTTGAGTGTCCTCCTTGTCCTCCCTGCCCTGCTC |
| | | CCGAGTTACTTGGCGGACCTTCAGTGTTCCTGTTCCCCCCCAAGCCCAA |
| | | GGATACTCTCATGATCAGCCGGACCCCCGAGGTCACCTGTGTGGTGGT |
| | | AGATGTTAGCCACGAGGACCCTGAGGTCAAGTTCAACTGGTACGTCGA |
| | | CGGCGTCGAGGTGCACAACGCCAAGACCAAGCCTCGTGAAGAACAGT |
| | | ACCAGTCCACCTACAGAGTTGTGAGCGTGCTTACCGTGCTGCACCAGG |
| | | ACTGGCTGAACGGCAAGGAGTACAAGTGCAAGGTGTCCAACAAGGCC |
| | | CTCCCCGCTCCCATCGAGAAGACAATCAGCAAGGCCAAGCCCTGTCCA |
| | | GCCCCTGAGCTCTTAGGAGGACCTAGCGTTTTCCTTTTCCCTCCCAAGC |
| | | CTAAGGACACTCTTATGATCTCCAGAACACCAGAGGTTACCTGCGTCGT |
| | | GGTGGACGTGTCCCATGAGGACCCAGAAGTCAAATTCAATTGGTATGT |
| | | AGATGGGGTCGAGGTCCACAACGCTAAGACAAAGCCCCGCGAGGAGC |
| | | AGTACAACTCTACCTACAGGGTCGTGTCCGTGCTCACAGTGCTGCATCA |
| | | GGATTGGCTCAACGGGAAGGAGTATAAGTGCAAAGTGTCCAATAAGGC |
| | | CCTTCCCGCCCCTATCGAGAAAACCATCTCTAAGGCCAAATTCTGGGTG |
| | | CTGGTGGTTGTGGGCGGCGTGCTTGCTTGTTACTCCCTGCTGGTCACTG |
| | | TAGCTTTCATCATATTTTGGGTG |

In embodiments, the hinge domain comprises a CD28 hinge region. In embodiments a CD28 hinge domain has the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 273 (IEVMYPPPYLDNEKSNGTIIHVKGKHLCPSPLFPGPSKP (SEQ ID NO: 273)). In embodiments, the hinge domain comprises a truncated CD28 hinge region (CD28T) hinge region, such as disclosed in International Patent Application No: PCT/US2017/025351, filed Mar. 31, 2017, which is incorporated herein by reference in its entirety. In embodiments the CARs described herein comprise a CD28T hinge domain having the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 274 (LDNEKSNGTIIHVKGKHLCPSPLFPGPSKP (SEQ ID NO: 274)).

In embodiments, the hinge domain comprises a CD8a hinge region. In embodiments the CARs described herein comprise a hinge domain from CD8a having the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 275 (TTTPAPRPPTPAPTIASQPLSLRPEACRPAAGGAVHTRGLDFACD (SEQ ID NO: 275)).

Polynucleotide and polypeptide sequences of these hinge domains are known. In some embodiments, the polynucleotide encoding a hinge domain comprises a nucleotide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% (e.g., 85-90%, 85-95%, 85-100%, 90-95%, 90-100%, or 95-100%) identical to a nucleotide sequence known. In some embodiments, the polypeptide sequence of a hinge domain comprises a polypeptide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% (e.g., 85-90%, 85-95%, 85-100%, 90-95%, 90-100%, or 95-100%) identical to a known polypeptide sequence.

In general, a “transmembrane domain” (e.g., of an antigen binding system) refers to a domain having an attribute of being present in the membrane when present in a molecule at a cell surface or cell membrane (e.g., spanning a portion or all of a cellular membrane). A costimulatory domain for an antigen binding system of the present disclosure may further comprise a transmembrane domain and/or an intracellular signaling domain. It is not required that every amino acid in a transmembrane domain be present in the membrane. For example, in some embodiments, a transmembrane domain is characterized in that a designated stretch or portion of a protein is substantially located in the membrane. Amino acid or nucleic acid sequences may be analyzed using a variety of algorithms to predict protein subcellular localization (e.g., transmembrane localization). The programs psort (PSORT.org) and Prosite (prosite.expasy.org) are exemplary of such programs.

The type of transmembrane domain comprised in an antigen binding system described herein is not limited to any type. In some embodiments, a transmembrane domain is selected that is naturally associated with a binding domain and/or intracellular domain. In some instances, a transmembrane domain comprises a modification of one or more amino acids (e.g., deletion, insertion, and/or substitution), e.g., to avoid binding of such domains to a transmembrane domain of the same or different surface membrane proteins to minimize interactions with other members of the receptor complex.

A transmembrane domain may be derived either from a natural or from a synthetic source. Where the source is natural, a domain may be derived from any membrane-bound or transmembrane protein. Exemplary transmembrane domains may be derived from (e.g., may comprise at least a transmembrane domain of) an alpha, beta or zeta chain of a T-cell receptor, 2B4, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CDS, CEACAM1, CRT AM, cytokine receptor, DAP-10, DAP-12, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof. In some embodiments, a transmembrane domain may be synthetic (and can, e.g., comprise predominantly hydrophobic residues such as leucine and valine). In some embodiments, a triplet of phenylalanine, tryptophan and valine are comprised at each end of a synthetic transmembrane domain. In some embodiments, a transmembrane domain is directly linked or connected to a cytoplasmic domain. In some embodiments, a short oligo- or polypeptide linker (e.g., between 2 and 10 amino acids in length) may form a linkage between a transmembrane domain and an intracellular domain. In some embodiments, a linker is a glycine-serine doublet.

In embodiments, the CARs described herein comprise a TM domain from CD28 having the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 276 (FWVLVVVGGVLACYSLLVTVAFIIFWV (SEQ ID NO: 276)).

In embodiments, the CARs described herein comprise a TM domain from CD8α having the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 277 (IYIWAPLAGTCGVLLLSLVITLYC (SEQ ID NO: 277)).

Polynucleotide and polypeptide sequences of transmembrane domains provided herein are known. In some embodiments, the polynucleotide encoding a transmembrane domain comprises a nucleotide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% (e.g., 85-90%, 85-95%, 85-100%, 90-95%, 90-100%, or 95-100%) identical to a nucleotide sequence known. In some embodiments, the polypeptide sequence of a transmembrane domain comprises a polypeptide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% (e.g., 85-90%, 85-95%, 85-100%, 90-95%, 90-100%, or 95-100%) identical to a polypeptide sequence known. Optionally, short spacers may form linkages between any or some of the extracellular, transmembrane, and intracellular domains of the CAR.

Intracellular signaling domains that may transduce a signal upon binding of an antigen to an immune cell are known, any of which may be comprised in an antigen binding system of the present disclosure. For example, cytoplasmic sequences of a T cell receptor (TCR) are known to initiate signal transduction following TCR binding to an antigen (see, e.g., Brownlie et al., Nature Rev. Immunol. 13:257-269 (2013)).

In some embodiments, CARs contemplated herein comprise an intracellular signaling domain. An “intracellular signaling domain,” refers to the part of a CAR that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. In some embodiments, a signaling domain and/or activation domain comprises an immunoreceptor tyrosine-based activation domain (ITAM). Examples of ITAM containing cytoplasmic signaling sequences comprise those derived from TCR zeta, FcR gamma, FcR beta, CD3 zeta, CD3 gamma, CD3 delta, CD3 epsilon, CD5, CD22, CD79a, CD79b, and CD66d (see, e.g., Love et al., Cold Spring Harb. Perspect. Biol. 2:a002485 (2010); Smith-Garvin et al., Annu. Rev. Immunol. 27:591-619 (2009)). In certain embodiments, suitable signaling domains comprise, without limitation, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

The term “effector function” refers to a specialized function of the cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. While usually the entire intracellular signaling domain may be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

It is known that signals generated through the TCR alone are insufficient for full activation of the T cell and that a secondary or costimulatory signal may also be required. Thus, T cell activation may be said to be mediated by two distinct classes of intracellular signaling domains: primary signaling domains that initiate antigen-dependent primary activation through the TCR (e.g., a TCR/CD3 complex) and costimulatory signaling domains that act in an antigen independent manner to provide a secondary or costimulatory signal. In some embodiments, a CAR contemplated herein comprises an intracellular signaling domain that comprises one or more “costimulatory signaling domain” and a “primary signaling domain.”

In some embodiments, a signaling domain and/or activation domain comprises an immunoreceptor tyrosine-based activation motif (ITAM). Examples of ITAM containing cytoplasmic signaling sequences comprise those derived from TCR zeta, FcR gamma, FcR beta, CD3 zeta, CD3 gamma, CD3 delta, CD3 epsilon, CD5, CD22, CD79a, CD79b, and CD66d (see, e.g., Love et al., Cold Spring Harb. Perspect. Biol. 2:a002485 (2010); Smith-Garvin et al., Annu. Rev. Immunol. 27:591-619 (2009)). In some embodiments, a CAR comprises a CD3ζ primary signaling domain and one or more costimulatory signaling domains. The intracellular primary signaling and costimulatory signaling domains may be linked in any order in tandem to the carboxyl terminus of the transmembrane domain. In one embodiment, the CARs have a CD3ζ domain having the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 278.

(SEQ ID NO: 278)
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPR.

CARs contemplated herein comprise one or more costimulatory signaling domains to enhance the efficacy and expansion of T cells expressing CAR receptors. As used herein, the term, “costimulatory signaling domain,” or “costimulatory domain”, refers to an intracellular signaling domain of a costimulatory molecule.

In certain embodiments, suitable signaling domains comprise, without limitation, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DAP-12, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

A CAR may comprise a costimulatory signaling domain, e.g., to increase signaling potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). Signals generated through a TCR alone may be insufficient for full activation of a T cell and a secondary or co-stimulatory signal may increase activation. Thus, in some embodiments, a signaling domain further comprises one or more additional signaling domains (e.g., costimulatory signaling domains) that activate one or more immune cell effector functions (e.g., a native immune cell effector function described herein). In some embodiments, a portion of such costimulatory signaling domains may be used, as long as the portion transduces the effector function signal. In some embodiments, a cytoplasmic domain described herein comprises one or more cytoplasmic sequences of a T cell co-receptor (or fragment thereof). Non-limiting examples of co-stimulatory domains include, but are not limited to, 4-1BB (also known as TNFRSF9, CD137, CDw137, ILA, and tumor necrosis factor receptor superfamily member 9), 4-1BBL/CD137, BAFFR, BLAME (SLAMF8), activating NK receptors, BTLA (also known as CD272 and BTLA1), CARD11, CD2 (also known as LFA-2, SRBC, T11, and CD2 molecule), CD3 gamma, CD3 delta, CD3 epsilon, CD4, CD7 (also known as GP40, LEU-9, TP41, Tp40, and CD7 molecule), CD8alpha, CD8beta, CD11a, CD11b, CD11c, CD11d, CD18, CD19, CD19a, CD27 (also known as S152, S152.LPFS2, T14, TNFRSF7, and Tp55), CD28 (also known as Tp44), CD29, CD30 (also known as TNFRSF8, D1S166E, and Ki-1), CD40L (also known as CD40LG, CD154, HIGM1, IGM, IMD3, T-BAM, TNFSF5, TRAP, gp39, hCD40L, and CD40 ligand), CD40 (also known as Bp50, CDW40, TNFRSF5, p50, CD40 (protein), and CD40 molecule), CD49a, CD49D, CD49f, CD54 (ICAM), CD69, CD80 (also known as B7, B7-1, B7.1, BB1, CD28LG, CD28LG1, LAB7, and CD80 molecule), CD83 (and a ligand that specifically binds with CD83), CD84, CD86, CD96 (Tactile), CD100 (SEMA4D), CD103, CD160 (also known as BY55, NK1, NK28, and CD160 molecule), CD244 (also known as 2B4, NAIL, NKR2B4, Nmrk, SLAMF4, and CD244 molecule), CD247, CD276 (also known as, B7-H3,4Ig-B7-H3, B7H3, B7RP-2), CD366, CDS, CEACAM1, CRT AM, cytokine receptors, DAP10, DNAM1 (CD226), Fc gamma receptor, GADS, GITR (also known as TNFRSF18, RP5-902P8.2, AITR, CD357, and GITR-D), GITRL, HVEM (also known as TNFRSF14, RP3-395M20.6, ATAR, CD270, HVEA, HVEM, LIGHTR, and TR2), ICAM-1, ICOS (also known as inducible T cell costimulatory, AILIM, CD278, and CVID1), Ig alpha (CD79a), IL2R beta, IL2R gamma, IL7R alpha, immunoglobulin-like proteins, integrins, ITGA4, IA4, ITGA6, ITGAD, ITGAE, ITGAL, ITGAM, ITGAX, ITGB 1, ITGB2, ITGB7, KIRDS2, LAGS (also known as CD223 and lymphocyte activating 3), LAT, LFA-1 (also known as Lymphocyte function-associated antigen 1 and CD11a/CD18), LIGHT (also known as TNFSF14, CD258, HVEML, LTg, TR2, TNLG1D, and tumor necrosis factor superfamily member 14), LTBR, Ly9 (CD229), MHC class I molecule, NKG2C (also known as CD314, D12S2489E, KLR, NKG2-D, NKG2D, and killer cell lectin like receptor K1), NKG2D, NKp30, NKp44, NKp46, NKp80 (KLRF1), OX40 (also known as TNFRSF4, ACT35, RP5-902P8.3, IMD16, CD134, TXGP1L, and tumor necrosis factor receptor superfamily member 4), PAG/Cbp, PD-1 (also known as PDCD1, CD279, PD-1, SLEB2, hPD-1, hPD-1, hSLE1, and Programmed cell death 1), PD-L1 (also known as CD274, B7-H, B7H1, PD-L1, PDCD1L1, PDCD1LG1, PDL1, CD274 molecule, and Programmed cell death 1 ligand 1), PSGL1, SELPLG (CD162), signaling lymphocytic activation molecules (SLAM proteins such as SLAM (SLAMF1, CD150, IPO-3), SLAMF4 (CD244, 2B4), SLAMF6 (NTB-A, Ly108), and SLAMF7), SLP76, TIM3 (also known as HAVCR2, HAVcr-2, KIM-3, TIM3, TIMD-3, TIMD3, Tim-3, and hepatitis A virus cellular receptor 2), TNF receptor proteins, TNFR2, Toll ligand receptor, TLR1, TLR2, TLR3, TLR4, TLR5, TLR6, TLR7, TLR8, TLR9, TLR10, TNFR2, TRANCE/RANKL, TRIM, VLA1, VLA-6, and ZAP70. An exemplary costimulatory protein has the amino acid sequence of a costimulatory protein found naturally on T cells, the complete native amino acid sequence of which costimulatory protein is described in NCBI Reference Sequence: NP_006130.1. In certain instances, a CAR comprises a 4-1BB costimulatory domain.

In embodiments, the CARs comprise a CD28 costimulatory domain having the amino acid sequence of having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 279. RSKRSRLLHSDYMNMTPRRPGPTRKHYQPYAPPRDFAAYRS (SEQ ID NO: 279). In embodiments, the CARs comprise a 4-IBB costimulatory domain having the amino acid sequence of having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 270.

(SEQ ID NO: 270)
RGRKKLLYIFKQPFMRPVQTTQEEDGCSCRFPEEEEGGCE

The engineered CARs described herein may also comprise an N-terminal signal peptide or tag at the N-terminus of the scFv or antigen binding domain. In one embodiment, a heterologous signal peptide may be used. The antigen binding domain or scFV may be fused to a leader or a signal peptide that directs the nascent protein into the endoplasmic reticulum and subsequent translocation to the cell surface. It is understood that, once a polypeptide containing a signal peptide is expressed at the cell surface, the signal peptide is generally proteolytically removed during processing of the polypeptide in the endoplasmic reticulum and translocation to the cell surface. Thus, a polypeptide such as the CAR constructs described herein, are generally expressed at the cell surface as a mature protein lacking the signal peptide, whereas the precursor form of the polypeptide includes the signal peptide. Any suitable signal sequence known in the art may be used. Similarly any known tag sequence known in the art may also be used. In one embodiment a signal sequence is a CSF2RA signal sequence. In some embodiments, an antigen binding system of the present disclosure comprises a leader peptide (also referred to herein as a “signal peptide” or “leader sequence”). In certain embodiments, a leader peptide comprises an amino acid sequence that is at least about 75%, at least about 80%, at least about 85%, at least about 90%, at least about 95%, at least about 96%, at least about 97%, at least about 98%, at least about 99%, or 100% (e.g., 85-90%, 85-95%, 85-100%, 90-95%, 90-100%, or 95-100%) identical to the amino acid sequence MEWTWVFLFLLSVTAGVHS (SEQ ID NO: 249), MALPVTALLLPLALLLHAARP (SEQ ID NO: 250), or MLLLVTSLLLCELPHPAFLLIP (SEQ ID NO: 280).

In various embodiments, a mechanism of modulating (e.g., decreasing) antigen binding system activity is desired, e.g., to minimize or curtail adverse events resulting from antigen binding system activity. It may also be desired to comprise an inducible “on” or “accelerator” switch in immune cells. Suitable techniques comprise use of inducible caspase-9 (U.S. Appl. 2011/0286980) or a thymidine kinase, before, after, or at the same time, as the cells are transduced with the CAR construct of the present disclosure. Additional methods for introducing suicide genes and/or “on” switches comprise TALENS, zinc fingers, RNAi, siRNA, shRNA, antisense technology, and other techniques.

In accordance with the present disclosure, on-off or other types of control switch techniques may be incorporated herein. These techniques may comprise use of dimerization domains and optional activators of such domain dimerization, e.g., as disclosed by Wu et al., Science 2014 350 (6258) utilizing FKBP/Rapalog dimerization systems in certain cells, the contents of which are incorporated by reference herein in their entirety. Additional dimerization technology is described in, e.g., Fegan et al. Chem. Rev. 2010, 110, 3315-3336 as well as U.S. Pat. Nos. 5,830,462; 5,834,266; 5,869,337; and 6,165,787, the contents of each of which is also incorporated by reference herein with respect to dimerization technology. Additional dimerization pairs may comprise cyclosporine-A/cyclophilin, receptor, estrogen/estrogen receptor (optionally using tamoxifen, 4-hydroxytamoxifen, or endoxifen), glucocorticoids/glucocorticoid receptor, tetracycline/tetracycline receptor, and/or vitamin D/vitamin D receptor. Further examples of dimerization technology may be found in e.g., WO 2014/127261, WO 2015/090229, US 2014/0286987, US 2015/0266973, US 2016/0046700, U.S. Pat. No. 8,486,693, US 2014/0171649, and US 2012/0130076, the contents of which are further incorporated by reference herein in their entirety.

Components of a CAR may be exchanged or “swapped” using routine techniques of biotechnology for equivalent components. To provide just a few non-limiting and partial examples, a CAR of the present disclosure may comprise a binding motif as provided herein in combination with a hinge provided herein and a costimulatory domain provided herein. In certain examples, a CAR of the present disclosure may comprise a leader sequence as provided herein together with a binding motif as provided herein in combination with a hinge provided herein and a costimulatory domain provided herein. In various embodiments, the present disclosure provides a binding motif according to any one of SEQ ID NOs: 251-260 in combination with (e.g., adjacently fused to) a hinge according to any one of SEQ ID NOs: 261-269, optionally in further combination with (e.g., adjacently fused to) a 41BB costimulatory domain according to SEQ ID NO: 270.

TABLE 18
Exemplary Binding motif sequences

| SEQ | VH/VL | |
|---|---|---|
| ID | Of | |
| NO | Table: | Sequence |
| 251 | Table 5 | ggtaccCCCGGgCCCATGGCTCTTCCTGTGACAGCTCTTCTGCTGCCCCTGGC |
| | (Abi) | CCTGCTTCTGCATGCTGCTAGACCTCAGGTTCAGTTGCAGCAATGGGGAG |
| | | CTGGCCTGTTAAAGCCCAGCGAAACCCTGTCCCTCACCTGCGCTGTGTAT |
| | | GGCGGAAGCTTCAGCGGCTATTACTGGAGCTGGATCCGGCAGCCTCCTG |
| | | GAAAAGGATTAGAATGGATCGGCGAGATAGACCACAGCGGGAGCACAA |
| | | ACTACAACCCCAGCCTGAAATCGCGGGTTACAATCTCTGTGGACACAAG |
| | | CAAGAATCAGTTCTCCCTGAAGCTGAGCAGCGTTACTGCCGCCGACACA |
| | | GCTGTGTACTATTGCGCCAGAGGCGGAGGCTCCTGGTACAGCAACTGGTT |
| | | CGATCCTTGGGGCCAAGGCACCATGGTGACCGTTTCCAGCGGCTCTACAA |
| | | GCGGCAGCGGGAAACCTGGTTCTGGAGAGGGCAGCACAAAGGGCGACA |
| | | TCCAGATGACACAGAGCCCCAGCACCCTTAGCGCCTCTGTGGGAGATAG |
| | | GGTTACCATTACCTGCAGGGCTTCCCAGAGCATCAGCAGCTGGCTGGCAT |
| | | GGTATCAACAGAAGCCTGGCAAGGCTCCCAAGCTGCTCATCTATGACGC |
| | | CTCCAGCCTGGAAAGCGGGGTTCCCTCCAGATTTAGCGGCTCAGGCTCCG |
| | | GAACAGAGTTCACCCTTACCATCTCTAGCCTGCAACCCGACGACTTCGCT |
| | | ACTTATTACTGTCAACAAGACAGAAGCTTGCCCCCCACATTCGGCGGAG |
| | | GGACCAAGGTTGAGATCAAG |
| 252 | Table 6 | ggtaccCCCGGgCCCATGGCTCTTCCTGTGACAGCTCTTCTGCTGCCCCTGGC |
| | (Ab2) | CCTGCTTCTGCATGCTGCTAGACCTCAGGTTCAGTTGCAGCAATGGGGAG |
| | | CTGGCCTGTTAAAGCCCAGCGAAACCCTGTCCCTCACCTGCGCTGTGTAT |
| | | GGCGGAAGCTTCAGCGGCATCCACTGGAACTGGATCCGGCAGCCTCCTG |
| | | GCAAAGGCCTTGAATGGATCGGCGATATCGACACCAGCGGCTCCACCAA |
| | | CTACAACCCCAGCCTGAAATCGAGGGTTACAATCTCTGTGGACACAAGC |
| | | AAGAATCAGTTCTCCCTGAAGCTGAGCAGCGTTACTGCCGCCGACACAG |
| | | CTGTGTACTATTGCGCCAGACTGGGCCAGGAAAGCGCTACCTACCTTGGC |
| | | ATGGATGTGTGGGGGCAGGGCACCACCGTTACTGTTAGCTCTGGCTCAAC |
| | | AAGCGGCAGCGGCAAGCCTGGCTCAGGAGAAGGAAGCACAAAGGGCGA |
| | | CATTGTAATGACTCAGAGCCCCGACAGCCTGGCCGTTAGCTTAGGCGAA |
| | | AGGGCTACAATCAATTGCAAGAGCAGCCAGAGCGTTCTGTACAGCAGCA |
| | | ACAACAAGAACTACCTCGCATGGTATCAACAGAAGCCAGGCCAGCCTCC |
| | | CAAGCTGCTCATCTACTGGGCTTCCACCAGAGAGAGCGGGGTTCCCGAT |
| | | AGATTCTCCGGCTCCGGTTCTGGAACAGATTTCACGCTCACAATCAGCAG |
| | | CTTACAGGCCGAGGATGTGGCTGTCTACTATTGTCAGCAGTTGTACACCT |
| | | ACCCCTTCACATTCGGCGGAGGCACCAAGGTTGAGATCAAG |
| 253 | Table 7 | ggtaccCCCGGgCCCATGGCTCTTCCTGTGACAGCTCTTCTGCTGCCCCTGGC |
| | (Ab3) | CCTGCTTCTGCATGCTGCTAGACCTCAGCTTCAGCTCCAAGAGAGCGGAC |
| | | CTGGCTTAGTGAAGCCCAGCGAAACCCTGTCCCTCACCTGCACCGTTTCT |
| | | GGCGGAAGCATCAGCAGCTCCAGCTATTACTGGGGATGGATCAGGCAGC |
| | | CCCCTGGCAAGGGTTTAGAATGGATCGGCTCGATATATTACTCCGGCAGC |
| | | ACCTACTATAACCCCAGCTTGAAGAGCCGGGTTACCATTTCTGTGGACAC |
| | | ATCAAAGAACCAGTTCAGCCTGAAGCTGAGCTCTGTGACTGCCGCCGAC |
| | | ACAGCTGTGTACTACTGTGCCAGAGAGACAGACTACTCCAGCGGCATGG |
| | | GCTACGGCATGGATGTGTGGGGACAAGGAACCACCGTTACTGTGAGCAG |
| | | CGGTTCCACCAGCGGCTCAGGCAAGCCTGGCTCAGGAGAAGGAAGCACC |
| | | AAGGGGGATATACAGATGACACAGAGCCCCTCCAGCCTGTCCGCCAGCG |
| | | TTGGCGATCGTGTAACGATCACCTGCCGGGCCTCTCAGAGCATCAACTCC |
| | | TACCTCAATTGGTATCAACAGAAGCCAGGCAAGGCCCCCAAATTACTCA |
| | | TCTACGCCGCCAGCAGCTTACAGAGCGGGGTTCCCTCTAGATTCTCCGGC |
| | | TCCGGTTCTGGAACAGATTTCACCCTCACTATCTCCAGCTTGCAGCCCGA |
| | | GGATTTCGCCACTTATTACTGTCAGCAGAGCCTGGCCGACCCCTTCACAT |
| | | TCGGCGGAGGCACAAAGGTTGAGATCAAG |
| 254 | Table 8 | ggtaccCCCGGgCCCATGGCTCTTCCTGTGACAGCTCTTCTGCTGCCCCTGGC |
| | (Ab4) | CCTGCTTCTGCATGCTGCTAGACCTCAGGTTCAGCTTGTGCAGAGCGGAG |
| | | CTGAAGTTAAGAAGCCTGGCGCCTCTGTGAAGGTTAGCTGCAAGGCCAG |
| | | CGGCTACACATTCAAGGAATATGGCATCTCCTGGGTTAGGCAGGCTCCCG |
| | | GCCAAGGCTTAGAATGGATGGGCTGGATCTCCGCCTACTCCGGCCACAC |
| | | CTACTACGCCCAGAAGCTTCAGGGCAGGGTTACCATGACCACCGACACC |
| | | AGCACCTCTACCGCCTATATGGAGCTGAGGAGCCTGAGATCGGACGACA |
| | | CAGCTGTGTATTACTGCGCCAGAGGCCCCCACTACGACGACTGGTCTGGA |
| | | TTTATCATCTGGTTCGACCCCTGGGGGCAGGGCACCCTGGTCACAGTTTC |
| | | TTCTGGCTCCACCAGCGGAAGCGGCAAGCCAGGCTCAGGCGAAGGATCT |
| | | ACAAAAGGCGACATCCAAATGACACAGAGCCCCAGCAGCTTGAGCGCCT |
| | | CCGTTGGCGACAGAGTTACAATCACCTGCAGGGCCTCTCAGAGCATCAG |
| | | CAGCTATTTGAATTGGTATCAACAGAAGCCAGGAAAGGCCCCTAAGCTG |
| | | CTCATCTACGCTGCCAGCTCGCTCCAATCTGGCGTTCCTAGCAGATTTAG |
| | | CGGCTCCGGCAGCGGCACAGACTTTACTCTTACCATTAGCTCCCTGCAGC |
| | | CCGAGGACTTCGCTACCTACTATTGCCAGCAAAGCTACAGATTCCCTCCC |
| | | ACCTTTGGCCAGGGCACAAAGGTTGAGATCAAG |
| 255 | Table 9 | ggtaccCCCGGgCCCATGGCTCTTCCTGTGACAGCTCTTCTGCTGCCCCTGGC |
| | (Ab5) | CCTGCTTCTGCATGCTGCTAGACCTCAGGTTCAGTTACAAGAGAGCGGAC |
| | | CTGGCTTAGTGAAGCCCAGCGAAACCCTGTCCCTCACCTGCACCGTTTCT |
| | | GGCGGAAGCATCAGCTCTCCCGACCATTACTGGGGATGGATCAGGCAGC |
| | | CCCCTGGCAAGGGTTTGGAATGGATCGGCAGCATCTACGCCAGCGGCAG |
| | | CACATTCTACAACCCCTCGCTCAAAAGCAGGGTTACTATTTCTGTGGACA |
| | | CAAGCAAAAATCAGTTCAGCCTGAAGCTGAGCTCTGTGACTGCCGCCGA |
| | | CACAGCTGTGTACTACTGTGCCAGAGAGACAGACTACTCCAGCGGGATG |
| | | GGCTACGGCATGGATGTGTGGGGACAAGGAACCACCGTTACTGTGAGCA |
| | | GCGGCTCCACAAGCGGCTCAGGCAAGCCTGGCTCAGGAGAAGGAAGCAC |
| | | CAAGGGGGACATTCAAATGACCCAAAGCCCCTCCAGCCTGTCCGCCAGC |
| | | GTTGGCGATAGGGTTACCATTACCTGCAGGGCCAGCCAAAGCATCAACT |
| | | CCTACCTAAATTGGTATCAACAGAAGCCAGGCAAGGCCCCCAAACTACT |
| | | CATTTACGCCGCCAGCAGCTTACAGAGCGGGGTTCCCTCTAGATTCTCCG |
| | | GCAGCGGTTCTGGAACAGATTTCACTCTCACAATATCTTCGCTGCAGCCC |
| | | GAGGATTTCGCTACCTACTATTGCCAGCAATCCCTGGCCGACCCCTTCAC |
| | | ATTCGGCGGAGGCACAAAGGTTGAGATCAAG |
| 256 | Table | ggtaccCCCGGgCCCATGGCTCTTCCTGTGACAGCTCTTCTGCTGCCCCTGGC |
| | 10 | CCTGCTTCTGCATGCTGCTAGACCTCAGATCACATTAAAAGAGAGCGGAC |
| | (Ab6) | CTACACTGGTGAAGCCCACCCAAACGCTTACCCTCACCTGCACCTTTAGC |
| | | GGGTTCAGCCTGGACACAGAGGGCGTTGGCGTTGGATGGATCAGGCAGC |
| | | CTCCTGGCAAAGCCCTCGAATGGCTTGCCCTCATCTACTTCAACGACCAG |
| | | AAGAGATACAGCCCCTCCTTAAAATCTCGGCTCACAATCACCAAAGACA |
| | | CAAGCAAAAATCAGGTTGTGCTCACCATGACCAACATGGACCCTGTGGA |
| | | CACCGCTGTGTACTACTGTGCCAGAGACACCGGCTACAGCAGATGGTAC |
| | | TACGGGATGGACGTTTGGGGCCAAGGCACCACTGTGACCGTTTCCAGCG |
| | | GCTCTACAAGCGGCAGCGGGAAACCTGGTTCTGGAGAGGGCAGCACAAA |
| | | GGGCGACATCCAGATGACGCAATCCCCCAGCTCTGTGAGCGCCTCTGTG |
| | | GGAGACAGAGTTACAATCACATGCCGGGCCTCCCAGGGCATCAGCTCTT |
| | | GGCTGGCATGGTATCAACAGAAGCCTGGCAAGGCTCCCAAGCTGCTCAT |
| | | CTATGCCGCCTCCTCCTTACAATCTGGAGTTCCCTCCAGGTTCAGCGGGA |
| | | GCGGCTCAGGAACAGACTTCACCCTTACCATCTCTAGCCTGCAACCCGAG |
| | | GACTTCGCTACTTATTACTGTCAGCAGGCCTACGCCTACCCCATCACATT |
| | | CGGCGGAGGAACAAAGGTTGAGATCAAG |
| 257 | Table | ggtaccCCCGGgCCCATGGCTCTTCCTGTGACAGCTCTTCTGCTGCCCCTGGC |
| | 11 | CCTGCTTCTGCATGCTGCTAGACCTCAGGTTCAGTTGCAGCAATGGGGAG |
| | (Ab7) | CTGGCCTGTTAAAGCCCAGCGAAACCCTGTCCCTCACCTGCGCTGTGTAT |
| | | GGCGGAAGCTTCGAGAAATACTACTGGAGCTGGATCCGGCAGCCTCCCG |
| | | GCAAAGGCTTAGAATGGATCGGCGAGATTTATCACAGCGGGCTCACCAA |
| | | CTACAACCCCAGCCTGAAATCTCGAGTTACAATCTCTGTGGACACAAGCA |
| | | AGAATCAGTTCTCCCTGAAGCTGAGCAGCGTTACTGCCGCCGACACAGCT |
| | | GTGTACTATTGCGCCAGAGTTAGATACGACAGCAGCGACAGCTATTACT |
| | | ACAGCTATGACTACGGCATGGATGTGTGGGGGCAGGGCACCACCGTTAC |
| | | TGTCTCCTCTGGATCTACCAGCGGCAGCGGCAAGCCTGGATCTGGCGAA |
| | | GGAAGCACAAAGGGCGACATTGTGCTCACCCAGAGCCCCGACAGCCTGG |
| | | CTGTGTCTTTAGGCGAAAGGGCTACCATCAACTGCAAGAGCAGCCAGAG |
| | | CGTTCTGTACAGCAGCAACAACAAGAACTACCTTGCTTGGTATCAACAG |
| | | AAGCCTGGCCAGCCCCCTAAGCTGCTCATCTACTGGGCCTCTAGCAGAGA |
| | | GAGCGGGGTTCCCGATCGGTTTAGCGGCTCCGGCTCAGGAACCGATTTCA |
| | | CCCTCACTATCTCCAGCCTCCAGGCCGAGGATGTGGCTGTCTACTATTGT |
| | | CAGCAGAGCTATAGCTTCCCCTGGACATTCGGCGGAGGCACCAAGGTTG |
| | | AGATCAAG |
| 258 | Table | ggtaccCCCGGgCCCATGGCTCTTCCTGTGACAGCTCTTCTGCTGCCCCTGGC |
| | 12 | CCTGCTTCTGCATGCTGCTAGACCTCAGGTTCAGTTACAACAATGGGGAG |
| | (Ab8) | CTGGCCTGTTAAAGCCCAGCGAAACCCTGTCCCTCACCTGCGCTGTGTAT |
| | | GGCGGAAGCTTCAGCCGCTATGTGTGGAGCTGGATCCGGCAGCCTCCTG |
| | | GCAAAGGCCTTGAATGGATCGGAGAGATAGACAGCAGCGGCAAGACCA |
| | | ACTACAACCCCAGCCTGAAATCACGCGTTACAATCTCTGTGGACACAAG |
| | | CAAGAATCAGTTCTCCCTGAAGCTGAGCAGCGTTACTGCCGCCGACACA |
| | | GCTGTGTACTATTGCGCCAGAGTTAGATACGACAGCTCCGACAGCTATTA |
| | | CTACAGCTATGACTACGGCATGGATGTGTGGGGGCAGGGCACCACCGTT |
| | | ACAGTTAGCTCTGGAAGCACCAGCGGCTCCGGCAAGCCTGGATCTGGTG |
| | | AAGGAAGCACAAAGGGCGACATTGTGCTCACCCAGAGCCCCGACAGCCT |
| | | GGCTGTGTCTTTAGGCGAAAGGGCTACCATCAACTGCAAGAGCAGCCAG |
| | | AGCGTTCTGTACAGCAGCAACAACAAGAACTACCTTGCATGGTATCAAC |
| | | AGAAGCCTGGCCAGCCTCCCAAGCTGCTCATCTACTGGGCCTCTAGCAGA |
| | | GAGAGCGGGGTTCCCGATCGCTTTAGCGGCAGCGGTTCTGGCACCGATTT |
| | | CACTCTTACAATCAGCAGCTTACAGGCCGAGGATGTGGCTGTCTACTATT |
| | | GTCAGCAGAGCTATAGCTTCCCCTGGACATTCGGCGGAGGCACCAAGGT |
| | | TGAGATCAAG |
| 259 | Table | ggtaccCCCGGgCCCATGGCTCTTCCTGTGACAGCTCTTCTGCTGCCCCTGGC |
| | 13 | CCTGCTTCTGCATGCTGCTAGACCTCAGGTTCAGTTACAACAATGGGGAG |
| | (Ab9) | CTGGCCTGTTAAAGCCCAGCGAAACCCTGTCCCTCACCTGCGCTGTGTAT |
| | | GGCGGAAGCTTCAGCGGCTACGCTTGGAGCTGGATTAGACAGCCTCCTG |
| | | GCAAAGGACTAGAATGGATCGGAGAGATCGACCACAGAGGCTTCACCAA |
| | | CTACAACCCCAGCCTGAAATCCAGAGTTACAATCTCTGTGGACACAAGC |
| | | AAGAATCAGTTCTCCCTGAAGCTGAGCAGCGTTACTGCCGCCGACACAG |
| | | CTGTGTACTATTGCGCCAGGGTTAGATACGACAGCAGCGACAGCTATTAC |
| | | TACAGCTATGACTACGGCATGGATGTGTGGGGGCAGGGCACCACCGTTA |
| | | CGGTTAGCTCTGGATCTACCAGCGGCAGCGGCAAGCCTGGCTCAGGAGA |
| | | AGGAAGCACAAAGGGCGACATTGTGCTCACCCAGAGCCCCGACAGCCTG |
| | | GCCGTTTCTTTAGGCGAAAGGGCTACCATCAACTGCAAGAGCAGCCAGA |
| | | GCGTTCTGTACAGCAGCAACAACAAGAACTACCTTGCATGGTATCAACA |
| | | GAAGCCAGGCCAGCCTCCCAAGCTGCTCATCTACTGGGCCTCTAGCAGA |
| | | GAGAGCGGGGTTCCCGATAGATTTTCGGGATCAGGCTCCGGCACCGATTT |
| | | CACTCTTACGATCAGCAGCTTACAGGCCGAGGATGTGGCTGTCTACTATT |
| | | GTCAGCAGAGCTATAGCTTCCCCTGGACATTCGGCGGAGGCACCAAGGT |
| | | TGAGATCAAG |
| 260 | Table | ggtaccCCCGGgCCCATGGCTCTTCCTGTGACAGCTCTTCTGCTGCCCCTGGC |
| | 14 | CCTGCTTCTGCATGCTGCTAGACCTCAGGTTCAGTTACAACAATGGGGAG |
| | (Ab 10) | CTGGCCTGTTAAAGCCCAGCGAAACCCTGTCCCTCACCTGCGCTGTGTAT |
| | | GGCGGAAGCTTCCAGAAATACTACTGGAGCTGGATCCGGCAGCCTCCCG |
| | | GCAAAGGCTTAGAATGGATCGGAGAGATAGACACCAGCGGCTTCACCAA |
| | | CTACAACCCCAGCCTGAAATCTAGGGTTACAATCTCTGTGGACACAAGC |
| | | AAGAATCAGTTCTCCCTGAAGCTGAGCAGCGTTACTGCCGCCGACACAG |
| | | CTGTGTACTATTGCGCCAGAGTTGGCAGATACAGCTACGGCTACTACATC |
| | | ACCGCCTTCGACATTTGGGGCCAAGGCACCACTGTGACCGTTTCCAGCGG |
| | | AAGCACTAGCGGCAGCGGGAAACCTGGTTCTGGAGAGGGCTCAACCAAG |
| | | GGCGACATCGTGATGACACAGAGCCCCGACTCTCTGGCTGTGTCCCTGGG |
| | | AGAGAGAGCCACCATCAACTGCAAGAGCAGCCAGAGCGTTCTGTACAGC |
| | | AGCAACAACAAGAACTACCTGGCATGGTATCAACAGAAGCCTGGCCAGC |
| | | CCCCTAAGCTGCTCATCTACTGGGCTTCCACCAGAGAATCAGGCGTTCCA |
| | | GACAGGTTCTCCGGCTCGGGTTCAGGCACAGACTTCACCCTTACCATCTC |
| | | TTCCCTGCAGGCCGAAGATGTGGCCGTTTACTACTGTCAGCAGCACTACA |
| | | GCTTCCCTTTCACATTCGGCGGAGGCACCAAGGTTGAGATCAAG |

A CAR linked or fused to an invariant TCR chain, such as an invariant TCRβ or invariant TCRα may comprise a CAR sequence and an invariant TCR sequence expressed as a single polypeptide comprising a cleavable linker between the invariant TCR and CAR. An exemplary cleavable linker is Furin-GSG-T2A (see, e.g., Chng et al. MAbs. 2015 March-April; 7(2): 403-412, which is herein incorporated by reference with respect to cleavable linkers; see also Guedan et al. Mol Ther Methods Clin Dev. 2019 Mar. 15; 12: 145-156, which is incorporated herein by reference.

Various CAR sequences, components, and/or frameworks are known, comprising without limitation sequences of hinges, spacers, transmembrane domains, costimulatory domains, stimulatory domains, binding motifs, and variants of each, and a CAR with desired binding and components or architecture can be readily constructed if, e.g., a heavy chain variable domain sequence or CDR sequences and a light chain variable domain sequence or CDR sequences are provided.

The present disclosure comprises nucleic acids encoding anti-CD20 binding motifs and/or anti-CD19 binding motifs provided herein. The present disclosure comprises nucleic acids encoding antibodies of the provided herein, comprising, without limitation, neucleic acids encoding binding motifs (e.g., anti-CD20 binding motifs and anti-CD19 binding motifs). The present disclosure comprises nucleic acids encoding duel targeting systems provided herein, comprising without limitation nucleic acids encoding CAR linked or fused to an invariant TCR chain, such as invariant TCRβ or invariant TCRα, for example an anti-CD19 CAR linked to invariant TCRβ or invariant TCRα and an anti-CD20 CAR linked to invariant TCRβ or invariant TCRα. The nucleic acid sequence of SEQ ID NO: 2 comprises and provides exemplary nucleic acid sequences corresponding to and encoding each of SEQ ID NOs: 1 and 3-11. The nucleic acid sequence of SEQ ID NO: 13 comprises and provides exemplary nucleic acid sequences corresponding to and encoding each of SEQ ID NOs: 12 and 14-22. The nucleic acid sequence of SEQ ID NO: 24 comprises and provides exemplary nucleic acid sequences corresponding to and encoding each of SEQ ID NOs: 23 and 25-33. The nucleic acid sequence of SEQ ID NO: 35 comprises and provides exemplary nucleic acid sequences corresponding to and encoding each of SEQ ID NOs: 34 and 36-44. The nucleic acid sequence of SEQ ID NO: 46 comprises and provides exemplary nucleic acid sequences corresponding to and encoding each of SEQ ID NOs: 45 and 47-55. The nucleic acid sequence of SEQ ID NO: 57 comprises and provides exemplary nucleic acid sequences corresponding to and encoding each of SEQ ID NOs: 56 and 58-66. The nucleic acid sequence of SEQ ID NO: 68 comprises and provides exemplary nucleic acid sequences corresponding to and encoding each of SEQ ID NOs: 67 and 69-77. The nucleic acid sequence of SEQ ID NO: 79 comprises and provides exemplary nucleic acid sequences corresponding to and encoding each of SEQ ID NOs: 78 and 80-88. The nucleic acid sequence of SEQ ID NO: 90 comprises and provides exemplary nucleic acid sequences corresponding to and encoding each of SEQ ID NOs: 89 and 91-99. The nucleic acid sequence of SEQ ID NO: 101 comprises and provides exemplary nucleic acid sequences corresponding to and encoding each of SEQ ID NOs: 100 and 102-110. The nucleic acid sequence of SEQ ID NO: 112 comprises and provides exemplary nucleic acid sequences corresponding to and encoding each of SEQ ID NOs: 111 and 113-121. The nucleic acid sequence of SEQ ID NO: 123 comprises and provides exemplary nucleic acid sequences corresponding to and encoding each of SEQ ID NOs: 122 and 124-132. The nucleic acid sequence of SEQ ID NO: 134 comprises and provides exemplary nucleic acid sequences corresponding to and encoding each of SEQ ID NOs: 133 and 135-143. The nucleic acid sequence of SEQ ID NO: 145 comprises and provides exemplary nucleic acid sequences corresponding to and encoding each of SEQ ID NOs: 144 and 146-154. The nucleic acid sequence of SEQ ID NO: 156 comprises and provides exemplary nucleic acid sequences corresponding to and encoding each of SEQ ID NOs: 155 and 157-165. The nucleic acid sequence of SEQ ID NO: 167 comprises and provides exemplary nucleic acid sequences corresponding to and encoding each of SEQ ID NOs: 166 and 168-176. The nucleic acid sequence of SEQ ID NO: 178 comprises and provides exemplary nucleic acid sequences corresponding to and encoding each of SEQ ID NOs: 177 and 179-187. The nucleic acid sequence of SEQ ID NO: 189 comprises and provides exemplary nucleic acid sequences corresponding to and encoding each of SEQ ID NOs: 188 and 190-198. The nucleic acid sequence of SEQ ID NO: 200 comprises and provides exemplary nucleic acid sequences corresponding to and encoding each of SEQ ID NOs: 199 and 201-209. The nucleic acid sequence of SEQ ID NO: 211 comprises and provides exemplary nucleic acid sequences corresponding to and encoding each of SEQ ID NOs: 210 and 212-220. The present disclosure comprises nucleic acids encoding anti-CD19 binding motifs provided herein. The nucleic acid sequence of SEQ ID NO: 222 comprises and provides exemplary nucleic acid sequences corresponding to and encoding each of SEQ ID NOs: 221 and 223-231. The nucleic acid sequence of SEQ ID NO: 233 comprises and provides exemplary nucleic acid sequences corresponding to and encoding each of SEQ ID NOs: 232 and 234-242.

The present disclosure comprises vectors that comprise nucleic acids of the present disclosure and/or that encode polypeptides of the present disclosure. In various embodiments, the present disclosure comprises a vector that comprises a nucleic acid encoding an anti-CD20 CAR linked to an invariant TCR, such as invariant TCRβ or invariant TCRα, and an anti-CD19 CAR linked to an invariant TCR, such as invariant TCRβ or invariant TCRα. In various embodiments, the present disclosure comprises one or more vectors that comprises a nucleic acid encoding an anti-CD20 CAR linked to an invariant TCR, such as invariant TCRβ or invariant TCRα, and an anti-CD19 CAR linked to an invariant TCR, such as invariant TCRβ or invariant TCRα.

Any vector may be suitable for the present disclosure. In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof. Suitable exemplary vectors include e.g., pGAR, pBABE-puro, pBABE-neo largeTcDNA, pBABE-hygro-hTERT, pMK0.1 GFP, MSCV-IRES-GFP, pMSCV PIG (Puro IRES GFP empty plasmid), pMSCV-loxp-dsRed-loxp-eGFP-Puro-WPRE, MSCV IRES Luciferase, pMIG, MDH1-PGK-GFP_2.0, TtRMPVIR, pMSCV-IRES-mCherry FP, pRetroX GFP T2A Cre, pRXTN, pLncEXP, and pLXIN-Luc.

A recombinant expression vector may be any suitable recombinant expression vector. Suitable vectors comprise those designed for propagation and expansion or for expression or both, such as plasmids and viruses. For example, a vector may be selected from the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also may be used. Examples of plant expression vectors useful in the context of the disclosure comprise pBI01, pBI101.2, pBI101.3, pBI121 and pBIN19 (Clontech). Examples of animal expression vectors useful in the context of the disclosure comprise pcDNA, pEUK-Cl, pMAM, and pMAMneo (Clontech).

Generally, it is understood that any appropriate viral vector may be used for transduction of the engineered constructs described herein. In one embodiment described herein, a cell (e.g., iNKT cell) is transduced with a retroviral vector, e.g., a lentiviral vector, encoding a construct as described herein.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in some embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis encephalitis virus (CAEV); equine infectious anemia virus (EIAV); feline immunodeficiency virus (FIV); bovine immune deficiency virus (BIV); and simian immunodeficiency virus (SIV).

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-retroviral viral sequences. In one embodiment, a hybrid vector refers to a vector or transfer plasmid comprising retroviral, e.g., lentiviral, sequences for reverse transcription, replication, integration and/or packaging.

In some embodiments, the terms “lentiviral vector,” “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles of the disclosure and are present in DNA form in the DNA plasmids of the disclosure. In one embodiment described herein, the expression vector is a lentivirus expression vector.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, Rand U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R, and U5. The U3 region contains the enhancer and promoter elements. The U5 region is the sequence between the primer binding site and the R region and contains the polyadenylation sequence. The R (repeat) region is flanked by the U3 and U5 regions. The LTR is composed of U3, R and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome which are required for insertion of the viral RNA into the viral capsid or particle, see e.g., Clever et al., 1995. J of Virology, Vol. 69, No. 4; pp. 2101-2109. Several retroviral vectors use the minimal packaging signal (also referred to as the psi [′P] sequence) needed for encapsidation of the viral genome. Thus, as used herein, the terms “packaging sequence,” “packaging signal,” “psi” and the symbol “′P,” are used in reference to the non-coding sequence required for encapsidation of retroviral RNA strands during viral particle formation.

In various embodiments, vectors comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment of the disclosure, the 3′LTR is modified such that the U5 region is replaced, for example, with an ideal poly(A) sequence. It should be noted that modifications to the LTRs such as modifications to the 3′LTR, the 5′LTR, or both 3′ and 5′LTRs, are also contemplated herein.

An additional safety enhancement is provided by replacing the U3 region of the 5′LTR with a heterologous promoter to drive transcription of the viral genome during production of viral particles. Examples of heterologous promoters which may be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter may be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR element. The term “TAR” refers to the “trans-activation response” genetic element located in the R region of lentiviral (e.g., HIV) LTRs. This element interacts with the lentiviral trans-activator (tat) genetic element to enhance viral replication.

The “R region” refers to the region within retroviral LTRs beginning at the start of the capping group (i.e., the start of transcription) and ending immediately prior to the start of the poly A tract. The R region is also defined as being flanked by the U3 and U5 regions. The R region plays a role during reverse transcription in permitting the transfer of nascent DNA from one end of the genome to the other.

As used herein, the term “FLAP element” refers to a nucleic acid whose sequence includes the central polypurine tract and central termination sequences (cPPT and CTS) of a includes the central polypurine tract and central termination sequences (cPPT and CTS) of a retrovirus, e.g., HIV-I or HIV-2. Suitable FLAP elements are described in U.S. Pat. No. 6,682,907 and in Zennou, et al., 2000, Cell, 101: 173. During HIV-I reverse transcription, central initiation of the plus-strand DNA at the central polypurine tract (cPPT) and central termination at the central termination sequence (CTS) lead to the formation of a three-stranded DNA structure: the HIV-I central DNA flap. While not wishing to be bound by any theory, the DNA flap may act as a cis-active determinant of lentiviral genome nuclear import and/or may increase the titer of the virus.

In one embodiment, retroviral or lentiviral transfer vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J Virol. 65: 1053; and Cullen et al., 1991. Cell 58: 423), and the hepatitis B virus post-transcriptional regulatory element (HPRE). Generally, the RNA export element is placed within the 3′ UTR of a gene, and may be inserted as one or multiple copies.

In other embodiments, expression of heterologous sequences in viral vectors is increased by incorporating post-transcriptional regulatory elements, efficient polyadenylation sites, and optionally, transcription termination signals into the vectors. A variety of posttranscriptional regulatory elements may increase expression of a heterologous nucleic acid at the protein, e.g., woodchuck hepatitis virus post-transcriptional regulatory element (WPRE; Zufferey et al., 1999, J Virol., 73:2886); the post-transcriptional regulatory element present in hepatitis B virus (HPRE) (Huang et al., Mol. Cell. Biol., 5:3864); and the like (Liu et al., 1995, Genes Dev., 9:1766).

In some embodiments, vectors may include regulatory oligonucleotides having transcriptional or translational regulatory activity. Such an oligonucleotide can be used in a variety of gene expression configurations for regulating control of expression. A transcriptional regulatory oligonucleotide, can increase (enhance) or decrease (silence) the level of expression of a recombinant expression construct. Regulatory oligonucleotides may selectively regulate expression in a context specific manner, including, for example, for conferring tissue specific, developmental stage specific, or the like expression of the polynucleotide, including constitutive or inducible expression. A regulatory oligonucleotide of the disclosure also can be a component of an expression vector or of a recombinant nucleic acid molecule comprising the regulatory oligonucleotide operatively linked to an expressible polynucleotide. A regulatory element can be of various lengths from a few nucleotides to several hundred nucleotides.

Elements directing the efficient termination and polyadenylation of the heterologous nucleic acid transcripts increases heterologous gene expression. Transcription termination signals are generally found downstream of the polyadenylation signal. In some embodiments, vectors comprise a polyadenylation sequence 3′ of a polynucleotide encoding a polypeptide to be expressed. The term “poly A site” or “poly A sequence” as used herein denotes a DNA sequence which directs both the termination and polyadenylation of the nascent RNA transcript by RNA polymerase II. Polyadenylation sequences may promote mRNA stability by addition of a poly A tail to the 3′ end of the coding sequence and thus, contribute to increased translational efficiency. Efficient polyadenylation of the recombinant transcript is desirable as transcripts lacking a poly A tail are unstable and are rapidly degraded. Illustrative examples of poly A signals that may be used in a vector of the disclosure, includes an ideal poly A sequence (e.g., AATAAA, ATTAAA, AGTAAA), a bovine growth hormone poly A sequence (BGHpA), a rabbit β-globin poly A sequence (rβgpA), or another suitable heterologous or endogenous poly A sequence known in the art.

Transgene promoters may include but are not limited to; MSCV, mPGK, EF1a and may also include iTCRa/b specific promotors. The iTCRa/b promoters will be provided by third party collaborator.

In some embodiments, to eliminate the potential for expression of the endogenous TCR complex the TCRa and/or TCR b genes may be knocked out or the expression reduced.

The present disclosure comprises cells that comprise, express, or are engineered (e.g., transformed or transduced) to comprise or express, at least one vector or nucleic acid of the present disclosure. In some embodiments, a method comprises transducing a cell with a vector that comprises a polynucleotide encoding at least one antigen binding system. The present disclosure comprises cells that comprise, or are transformed to comprise, at least one vector that encodes one or more polypeptides of the present disclosure. The present disclosure comprises cells that comprise, or are transformed to comprise, at least one vector, such as two vectors, that encode an anti-CD20 CAR linked to an invariant TCR, such as invariant TCRβ or invariant TCRα, and an anti-CD19 CAR linked to an invariant TCR, such as invariant TCRβ or invariant TCRα. In some embodiments, cells are co-transfected or co-transduced with two vectors, each vector encoding a different CARs linked to an invariant TCR, such as invariant TCRβ or invariant TCRα, which two different CARs together are the dual targeting system. Transfection or transduction of cells with two different vectors encoding two different CARs linked to linked to an invariant TCR, such as invariant TCRβ or invariant TCRα that together are the dual targeting system may be performed simultaneously on a single population of cells, simultaneously on two different populations of cells with each population transduced with only one of the two vectors, or independently on two different populations of cells each transduced with only one of the two vectors.

The present disclosure comprises cells that comprise one or more polypeptides of the present disclosure. The present disclosure comprises cells that comprise (e.g., express) an anti-CD20 CAR linked to an invariant TCR, such as invariant TCRβ or invariant TCRα, and an anti-CD19 CAR linked to an invariant TCR, such as invariant TCRβ or invariant TCRα.

In other aspects, provided herein are cells comprising a polynucleotide or a vector of the present disclosure. In some embodiments, the present disclosure is directed to cells, e.g., in vitro cells, comprising a polynucleotide I encoding two different CARs linked to linked to an invariant TCR, such as invariant TCRβ or invariant TCRα, for example an anti-CD20 CAR linked to an invariant TCR, such as invariant TCRβ or invariant TCRα, and an anti-CD19 CAR linked to an invariant TCR, such as invariant TCRβ or invariant TCRα.

Any cell may be used as a host cell for the polynucleotides, the vectors, or the polypeptides of the present disclosure. In some embodiments, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium, Serratia, e.g., Serratia marcescans, and Shigella; Bacilli such as B. subtilis and B. licheniformis; Pseudomonas such as P. aeruginosa; and Streptomyces. In some embodiments, the cell is a human cell. In some embodiments, the cell is an immune cell. In some embodiments, the immune cell is selected from the group consisting of a T cell, a B cell, a tumor infiltrating lymphocyte (TIL), a TCR expressing cell, a natural killer (NK) cell, iNKT cell, a dendritic cell, a granulocyte, an innate lymphoid cell, a megakaryocyte, a monocyte, a macrophage, a platelet, a thymocyte, and a myeloid cell. In one embodiment, the immune cell is a T cell. In another embodiment, the immune cell is an NK cell. In certain embodiments, the T cell is a tumor-infiltrating lymphocyte (TIL), autologous T cell, engineered autologous T cell (eACT™), an allogeneic T cell, a heterologous T cell, or any combination thereof.

Chimeric antigen receptors (CARs) linked to invariant TCRs may be readily inserted into and expressed by immune cells, e.g., iNKT cells, producing binding agents. In certain embodiments the iNKT cells are grown from culture (see for example Zhu et al., Development of Hematopoietic Stem Cell-Engineered Invariant Natural Killer T Cell Therapy for Cancer, Cell Stem Cell (2019), https://doi.org/10.1016/j.stem.2019.08.004). In certain embodiments, cells are obtained from a donor subject. In some embodiments, the donor subject is human patient afflicted with a cancer or a tumor. In other embodiments, the donor subject is a human patient not afflicted with a cancer or a tumor. In some embodiments, an engineered cell is autologous to a subject. In some embodiments, an engineered cell is allogeneic to a subject. The cell of the present disclosure may be obtained through any source known in the art. For example, the iNKT cells can be differentiated in vitro from a hematopoietic stem cell population, or can be obtained from a subject.

In some embodiments, the immune cells, e.g., iNKT cells, are genetically modified following isolation using known methods, or the immune cells are activated and expanded (or differentiated in the case of progenitors) in vitro prior to being genetically modified. In another embodiment, the immune cells, e.g., iNKT cells, are genetically modified with the chimeric antigen receptors described herein (e.g., transduced with a viral vector comprising one or more nucleotide sequences encoding a CAR linked to an invariant TCR, such as invariant TCRα or invariant TCRβ) and then are activated and/or expanded in vitro.

Other aspects of the present disclosure are directed to compositions comprising a polynucleotide described herein, a vector described herein, a polypeptide described herein, or an in vitro cell described herein. In some embodiments, the composition comprises a pharmaceutically acceptable carrier, diluent, solubilizer, emulsifier, preservative and/or adjuvant. In some embodiments, the composition comprises an excipient.

In other embodiments, the composition is selected for parenteral delivery, for inhalation, or for delivery through the digestive tract, such as orally. The preparation of such pharmaceutically acceptable compositions is within the ability of one skilled in the art. In certain embodiments, buffers are used to maintain the composition at physiological pH or at a slightly lower pH, typically within a pH range of from about 5 to about 8. In certain embodiments, when parenteral administration is contemplated, the composition is in the form of a pyrogen-free, parenterally acceptable aqueous solution comprising a composition described herein, with or without additional therapeutic agents, in a pharmaceutically acceptable vehicle. In certain embodiments, the vehicle for parenteral injection is sterile distilled water in which composition described herein, with or without at least one additional therapeutic agent, is formulated as a sterile, isotonic solution, properly preserved. In certain embodiments, the preparation involves the formulation of the desired molecule with polymeric compounds (such as polylactic acid or polyglycolic acid), beads or liposomes, that provide for the controlled or sustained release of the product, which are then be delivered via a depot injection. In certain embodiments, implantable drug delivery devices are used to introduce the desired molecule.

In some embodiments, the present disclosure provides pharmaceutical compositions that comprise and/or deliver one or more of the present disclosure, e.g., an antigen binding systems of the present disclosure, nucleic acids that encode them, and/or cell(s) or populations thereof that comprise and/or express them.

In some embodiments, the present disclosure provides pharmaceutical compositions that comprise and or deliver one or more cells as provided herein. A pharmaceutical composition may comprise one or a plurality of cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

A pharmaceutical composition of the present disclosure may be formulated for administration according to any embodiment set forth herein, at least one non-limiting example of which is intravenous administration. A composition may be formulated for intravenous, intratumoral, intraarterial, intramuscular, intraperitoneal, intrathecal, epidural, and/or subcutaneous administration routes. Preferably, the composition is formulated for a parenteral route of administration. A composition suitable for parenteral administration may be an aqueous or nonaqueous, isotonic sterile injection solution, which may contain antioxidants, buffers, bacteriostats, and solutes, for example, that render the composition isotonic with the blood of the intended recipient. An aqueous or nonaqueous sterile suspension may contain one or more suspending agents, solubilizers, thickening agents, stabilizers, and preservatives. Binding agent pharmaceutical compositions of the present disclosure may be administered in a manner appropriate to the disease to be treated (or prevented).

The sterile composition for injection may be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

Non-limiting examples of oily liquid comprise sesame oil and soybean oil, and it may be combined with benzyl benzoate or benzyl alcohol as a solubilizing agent. Other items that may be comprised are a buffer such as a phosphate buffer, or sodium acetate buffer, a soothing agent such as procaine hydrochloride, a stabilizer such as benzyl alcohol or phenol, and an antioxidant. The formulated injection may be packaged in a suitable ampule.

In one embodiment, a pharmaceutical composition is substantially free of detectable levels of a contaminant, e.g., of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenzae, Neisseria meningitides, Pseudomonas aeruginosa, Staphylococcus aureus, Streptococcus pneumonia, and/or Streptococcus pyogenes group A.

In various embodiments, cells provided herein (e.g., iNKT cells) may be activated and/or expanded from, and/or to produce, a pharmaceutical composition. In some embodiments, additional steps may be performed prior to administration to a subject. For instance, expanded in vitro after contacting (e.g., transducing or transfecting) an immune cell with an inducible expression construct or a constitutive expression construct described herein (e.g., an expression vector comprising an inducible expression construct or a constitutive expression construct), prior to the administration to a subject. In vitro expansion may proceed for 1 day or more, e.g., 2 days or more, 3 days or more, 4 days or more, 6 days or more, or 8 days or more, prior to the administration to a subject. In some embodiments, in vitro expansion may proceed for 21 days or less, e.g., 18 days or less, 16 days or less, 14 days or less, 10 days or less, 7 days or less, or 5 days or less, prior to administration to a subject. For example, in vitro expansion may proceed for 1-7 days, 2-10 days, 3-5 days, or 8-14 days prior to the administration to a subject. A binding agent pharmaceutical composition comprising, e.g., binding agents (e.g., engineered iNKT cells), may be formulated for administration at a desired dosage, e.g., a dosage of 10⁴ to 10⁹ cells/kg body weight (e.g., 10⁵ to 10⁶ cells/kg body weight).Certain embodiments of the disclosure comprise methods of administering to a subject a pharmaceutical composition as described herein, such as, for example, a binding agent described (e.g., a population of engineered cells of the present disclosure) herein, a protein therapeutic described herein, a composition comprising an engineered iNKT cell, e.g., in an amount effective to treat a subject, when administered in an appropriate dosing regimen.

In certain embodiments, an engineered iNKT cell is administered prior to, substantially simultaneously with, or after the administration of another therapeutic agent, such as a cancer therapeutic agent. The cancer therapeutic agent may be, e.g., a chemotherapeutic agent, a biological agent, or radiation treatment. In some embodiments, a subject receiving a engineered iNKT cell is not administered a treatment which is sufficient to cause a depletion of immune cells, such as lymphodepleting chemotherapy or radiation therapy.

Dosage administered to a subject in some embodiments, may vary with the embodiment, the composition employed, the method of administration, and the site and subject being treated. However, a dose should be sufficient to provide a therapeutic response. A clinician may determine the therapeutically effective amount of a composition to be administered to a human or other subject in order to treat or prevent a medical condition. The precise amount of the composition required to be therapeutically effective may depend upon numerous factors, e.g., such as the activity of the engineered iNKT cell, and the route of administration.

A suitable number engineered iNKT cell cells may be administered to a subject. While a single engineered iNKT cell described herein is capable of expanding and providing a therapeutic benefit, in some embodiments, 10² or more, e.g., 10³ or more, 10⁴ or more, 10⁵ or more, or 10⁸ or more, engineered iNKT cell cells are administered. In some embodiments, 10¹² or less, e.g., 10¹¹ or less, 10⁹ or less, 10⁷ or less, or 10⁵ or less, engineered iNKT cell cells described herein are administered to a subject. In some embodiments, 10²-10⁵, 10⁴-10⁷, 10³-10⁹, or 10⁵-10¹⁰ engineered iNKT cell cells described herein are administered. An engineered iNKT cell pharmaceutical composition may be administered, e.g., a dosage of 10⁴ to 10⁹ cells/kg body weight (e.g., 10⁵ to 10⁶ cells/kg body weight). A engineered iNKT cell pharmaceutical composition may be administered at a dosage of, e.g., about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

A dose of a engineered iNKT cell described herein may be administered to a mammal at one time or in a series of subdoses administered over a suitable period of time, e.g., on a daily, semi-weekly, weekly, bi-weekly, semi-monthly, bi-monthly, semi-annual, or annual basis, as needed. A dosage unit comprising an effective amount of an engineered iNKT cell may be administered in a single daily dose, or the total daily dosage may be administered in two, three, four, or more divided doses administered daily, as needed.

A suitable means of administration may be selected by a medical practitioner. Route of administration may be parenteral, for example, administration by injection, transnasal administration, transpulmonary administration, or transcutaneous administration. Administration may be systemic or local by intravenous injection, intramuscular injection, intraperitoneal injection, subcutaneous injection. In some embodiments, a composition is selected for parenteral delivery, for inhalation, or for delivery through the digestive tract, such as orally. Dose and method of administration may vary depending on the weight, age, condition, and the like of the subject, and may be suitably selected.

In various embodiments, a composition comprising a engineered iNKT cell as described herein, e.g., a sterile formulation for injection, may be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like. As disclosed herein, a pharmaceutical composition comprising an engineered iNKT cell may be in any form. Such forms comprise, e.g., liquid, semi-solid and solid dosage forms, such as liquid solutions (e.g., injectable and infusible solutions), dispersions or suspensions, tablets, pills, powders, liposomes and suppositories.

Selection or use of any form may depend, in part, on the intended mode of administration and therapeutic application. For example, a composition comprising a engineered iNKT cell of the present disclosure intended for systemic or local delivery may be in the form of injectable or infusible solutions. Accordingly, the compositions comprising a engineered iNKT cell of the present disclosure may be formulated for administration by a parenteral mode (e.g., intravenous, subcutaneous, intraperitoneal, or intramuscular injection). Parenteral administration refers to modes of administration other than enteral and topical administration, usually by injection, and comprise, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

Route of administration may be parenteral, for example, administration by injection, transnasal administration, transpulmonary administration, or transcutaneous administration. Administration may be systemic or local by intravenous injection, intramuscular injection, intraperitoneal injection, subcutaneous injection.

A pharmaceutical solution may comprise a therapeutically effective amount of a composition comprising an engineered iNKT cell of the present disclosure. Such effective amounts may be readily determined based, in part, on the effect of the administered composition comprising an engineered iNKT cell of the present disclosure, or the combinatorial effect of the composition comprising an engineered iNKT cell of the present disclosure and one or more additional active agents, if more than one agent is used. A therapeutically effective amount of a composition comprising a engineered iNKT cell of the present disclosure may also vary according to factors such as the disease state, age, sex, and weight of the individual, and the ability of the composition (and one or more additional active agents) to elicit a desired response in the individual, e.g., amelioration of at least one condition parameter, e.g., amelioration of at least one symptom of the complement-mediated disorder. For example, a therapeutically effective amount of a composition comprising an engineered iNKT cell of the present disclosure may inhibit (lessen the severity of or eliminate the occurrence of) and/or prevent a disorder, and/or any one of the symptoms of the disorder. A therapeutically effective amount is also one in which any toxic or detrimental effects of the composition comprising a engineered iNKT cell of the present disclosure are outweighed by the therapeutically beneficial effects.

Suitable human doses of any of the compositions comprising an engineered iNKT cell of the present disclosure may further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

The present disclosure provides technologies for simultaneously targeting CD20 and CD19. In some embodiments, the present disclosure provides technologies for initiating and/or modulating immune responses. In some embodiments, the present disclosure provides technologies for treating cancer (e.g., cancer characterized by cell(s) with surface-expressed CD20).

The present specification comprises use of a pharmaceutical composition provided herein to treat or prevent cancer. Another aspect of the present disclosure is directed to a method for treating or preventing a malignancy, said method comprising administering to a subject in need thereof an effective amount of pharmaceutical composition, e.g., where the cells comprises at least one Antigen binding system provided herein. Methods of the present disclosure comprising administration of an pharmaceutically effective amount of pharmaceutical composition of the present disclosure may be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In certain embodiments, a method provided herein induces a complete response. In some embodiments, a method provided herein induces a partial response. In certain embodiments the binding agent pharmaceutical composition is, comprises, comprises as the active agent, or comprises as the sole active agent, cells provided herein, e.g., cells that comprise they comprise a first CAR linked to a first invariant TCR chain, such as an invariant TCRα chain and a second CAR linked to a second invariant TCR chain, such as an invariant TCRβ chain. In some embodiments, a linker, such as a cleavable linker, for example a P2A or T2A, may be used to connect or link the CAR and the invariant TCR chain, such as an invariant TCRα or invariant TCRβ chain. In one example a CAR is linked to an invariant TCRα chain. In another example, CAR is linked to an invariant TCRβ chain. In certain embodiments, one CAR is linked to an invariant TCRα chain and second CAR (in the same cell) is linked to an invariant TCRβ chain. In certain embodiments, an anti-CD19 CAR is linked to an invariant TCRα chain and anti-CD20 CAR (in the same cell) is linked to an invariant TCRβ chain. In certain embodiments, an anti-CD20 CAR is linked to an invariant TCRα chain and anti-CD19 CAR (for example, in the same cell) is linked to an invariant TCRβ chain. In various embodiments, one or more binding domain or motifs of the CARs are scFv.

In various embodiments, the present disclosure comprises use of engineered iNKT cells provided herein to induce in a subject, or provide a subject with, immunity against a cancer. The present disclosure further comprises a method of preventing cancer in a subject by administering to the subject pharmaceutical composition provided herein. The present disclosure further comprises a method of inducing an immune response in a subject by administering to the subject pharmaceutical composition provided herein. In certain embodiments the binding agent pharmaceutical composition is, comprises, comprises as the active agent, or comprises as the sole active agent, cells provided herein.

The iNKT cells can be administered at a therapeutically effective amount. For example, a therapeutically effective amount of the iNKT cells can be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the iNKT cells about 10⁴ cells, about 10⁵ cells, about 10⁶ cells, about 10⁷ cells, or about 10⁸ cells. In one particular embodiment, the therapeutically effective amount of the iNKT cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

The methods of the disclosure can be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In certain embodiments, the methods induce a complete response. In other embodiments, the methods induce a partial response.

In certain embodiments, the cancer comprises cells that express CD19, e.g., on the surface of the cell. In certain embodiments the cancer comprises cells that express CD20, e.g., on the surface of the cell. In certain embodiments the cancer comprises cells that each individually express both CD19 and CD20, e.g., on the surface of the cell.

Cancers that may be treated include tumors that are not vascularized, not yet substantially vascularized, or vascularized. The cancer may also include solid or non-solid tumors. In some embodiments, the cancer is a hematologic cancer. In some embodiments, the cancer is of the white blood cells. In other embodiments, the cancer is of the plasma cells. In some embodiments, the cancer is leukemia, lymphoma, or myeloma. In certain embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, diffuse large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBC), small cell- or a large cell-follicular lymphoma, splenic marginal zone lymphoma (SMZL), systemic amyloid light chain amyloidosis, T-cell acute lymphoid leukemia (“TALL”), T-cell lymphoma, transformed follicular lymphoma, Waldenstrom macroglobulinemia, or a combination thereof. In one embodiment, the cancer is a myeloma. In one particular embodiment, the cancer is multiple myeloma. In another embodiment, the cancer is a leukemia. In one embodiment, the cancer is acute myeloid leukemia.

In various embodiments, a cell therapy provided herein for use in the present disclosure may be administered to a subject in a course of treatment that further comprises administration of one or more additional therapeutic agents or therapies that are not a cell therapy provided herein. In certain embodiments, the present disclosure provides combination therapy for the treatment of cancer, the treatment comprising administering an anti-cancer agent to a subject receiving and/or in need thereof.

In certain embodiments, administration of an engineered iNKT cell provided herein may be to a subject having previously received, scheduled to receive, or in the course of a treatment regimen comprising an additional anti-cancer therapy. In various embodiments, an additional agent or therapy administered in combination with an engineered iNKT cell provided herein as described herein may be administered at the same time as binding agent provided herein, on the same day as binding agent provided herein, or in the same week as binding agent provided herein. In various embodiments, an additional agent or therapy administered in combination with an engineered iNKT cell provided herein as described herein may be administered such that administration of the binding agent provided herein and the additional agent or therapy are separated by one or more hours before or after, one or more days before or after, one or more weeks before or after, or one or more months before or after administration of binding agent provided herein. In various embodiments, the administration frequency of one or more additional agents may be the same as, similar to, or different from the administration frequency of an engineered iNKT cell provided herein.

An agent or therapy used in combination with binding agent provided herein may be administered in a single therapeutic composition or dose together with binding agent provided herein, at the same time as binding agent provided herein in the form of a separate composition, or in a manner temporally distinct from the administration of binding agent provided herein. When an engineered iNKT cell provided herein is to be used in combination with an additional agent, the binding agent provided herein may be co-formulated with the additional agent or the binding agent provided herein may be formulated separately from the additional agent formulation.

In some embodiments, the methods further comprise administering a chemotherapeutic. In certain embodiments, the chemotherapeutic selected is a lymphodepleting (preconditioning) chemotherapeutic. Beneficial preconditioning treatment regimens, along with correlative beneficial biomarkers are described in U.S. Provisional Patent Applications 62/262,143 and 62/167,750 which are hereby incorporated by reference in their entirety herein. These describe, e.g., methods of conditioning a patient in need of a T cell therapy comprising administering to the patient specified beneficial doses of cyclophosphamide (between 200 mg/m²/day and 2000 mg/m²/day) and specified doses of fludarabine (between 20 mg/m²/day and 900 mg/m²/day). One such dose regimen involves treating a patient comprising administering daily to the patient about 500 mg/m²/day of cyclophosphamide and about 60 mg/m²/day of fludarabine for three days prior to administration of a therapeutically effective amount of engineered iNKT cells to the patient. In other embodiments, the antigen binding molecule, transduced (or otherwise engineered) cells, and the chemotherapeutic agent are administered each in an amount effective to treat the disease or condition in the subject.

In certain embodiments, compositions comprising iNKT cells disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. In some embodiments, compositions comprising iNKT cells disclosed herein may be administered in conjunction with an anti-hormonal agent that acts to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In some embodiments, the chemotherapeutic agent is administered at least 1 month before administering the cell or nucleic acid. In some embodiments, the methods further comprise administering two or more chemotherapeutic agents.

A variety of additional therapeutic agents may be used in conjunction with the compositions described herein. For example, potentially useful additional therapeutic agents include PD-1 inhibitors such as nivolumab)(OPDIVO®), pembrolizumab (KEYTRUDA®), pembrolizumab, pidilizumab (CureTech), and atezolizumab (Roche). Additional therapeutic agents suitable for use in combination with the disclosure include, but are not limited to, ibrutinib (IMBRUVICA®), ofatumumab (ARZERRA®), rituximab (RITUXAN®), bevacizumab (AVASTIN®), trastuzumab (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In additional embodiments, the composition comprising iNKT cells are administered with an anti-inflammatory agent. Anti-inflammatory agents or drugs can include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular), and minocycline.

In certain embodiments, the compositions described herein are administered in conjunction with a cytokine. “Cytokine” is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines. In various embodiments, an engineered iNKT cell provided herein for use in the present disclosure may be administered to a subject in a course of treatment that further comprises administration of an anti-inflammatory agent. Anti-inflammatory agents may comprise, without limitation, steroids and glucocorticoids (comprising betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) comprising aspirin, ibuprofen, naproxen, methotrexate, sulfasalazine, leflunomide, anti-TNF medications, cyclophosphamide and mycophenolate. Exemplary NSAIDs comprise ibuprofen, naproxen, naproxen sodium, Cox-2 inhibitors, and sialylates. Exemplary analgesics comprise acetaminophen, oxycodone, tramadol of proporxyphene hydrochloride. Exemplary glucocorticoids comprise cortisone, dexamethasone, hydrocortisone, methylprednisolone, prednisolone, or prednisone. Exemplary biological response modifiers comprise molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers comprise monoclonal antibodies as well as recombinant forms of molecules. Exemplary DMARDs comprise azathioprine, cyclophosphamide, cyclosporine, methotrexate, penicillamine, leflunomide, sulfasalazine, hydroxychloroquine, Gold (oral (auranofin) and intramuscular), and minocycline.

In various embodiments, an engineered iNKT cell provided herein for use in the present disclosure may be administered to a subject in a course of treatment that further comprises administration of a CHOP. CHOP consists of (C)yclophosphamide, an alkylating agent which damages DNA by binding to it and causing the formation of cross-links; (H)ydroxydaunorubicin (also called doxorubicin or adriamycin), an intercalating agent which damages DNA by inserting itself between DNA bases; (O)ncovin (vincristine), which prevents cells from duplicating by binding to the protein tubulin; and (P)rednisone or (P)rednisolone, which are corticosteroids.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control. The contents of all references cited throughout this application are expressly incorporated herein by reference.

### EXAMPLES

### Example 1

Invariant natural killer T cells (iNKT) are a small population of CD3+ve TCR positive NK cells that recognize glycolipid presented by CD1d. Glycolipid recognition is mediated by an invariant TCR αβ pair.

Introduction of the invariant TCR into HSCs results in iNKTs that are 100% positive for the TCR which drives the iNKT differentiation. Using the invariant TCR αβ genes as drivers co-transduced dual targeting CAR constructs is done by splitting the TCR α and β genes into 2 vectors each of which encodes one CAR gene. Only HSCs that receive both the TCR α-CAR-A and TCR β-CAR-B construct will express the full invariant TCR. iNKT differentiation will then only be driven by cells that are TCR αβ positive and will therefore be CAR-A CAR-B positive.

Generation of iNKT Dual CAR CD19 and CD20.

CD19 and CD20 CAR constructs are split into two viral vectors along with the invariant TCR alpha and beta chains. The CAR constructs are separated from the TCR coding sequences by a 2A self-cleaving peptide sequence. In general, the two constructs will have the following architecture.

Leader Peptide, Coding Sequence A, 2A, Coding Sequence B.

Both genetic constructs will be delivered to iNKT precursor cells either CD34+Ve HSC's, iPSC's or other appropriate precursor cell types by viral transduction. Genetic constructs will delivered by lentivirus, however other transduction methods are envisioned including gamma retrovirus, adeno associated virus and non-viral delivery. Lentiviral delivery will result in random integration of both transgenes however targeted integration using AAV and Homology directed repair into the TCR α and β locus is also contemplated. Preparation of viral vectors will use standard protocols and methods. iNKT cells will be differentiated in vitro using appropriate methods and reagents.

1. An antigen binding system comprising (i) a first construct comprising a first chimeric antigen receptor (CAR) linked to a first invariant T-cell receptor (TCR) and (ii) a second construct comprising a second CAR linked to a second invariant TCR.

2. The antigen binding system of claim 1, wherein the first invariant TCR is an invariant TCRα chain and the second invariant TCR is an invariant TCRβ chain.

3. The antigen binding system of claim 1,
(i) wherein the first construct comprises in the order of N-terminus to C-terminus (i) an optional leader peptide, (ii) an invariant TCRα chain, (iii) a linker, and (iv) the first CAR, and wherein the second construct comprises in the order of N-terminus to C-terminus (i) an optional leader peptide, (ii) an invariant TCRβ chain, (iii) a linker, and (iv) the second CAR; or
(ii) wherein the first construct comprises in the order of N-terminus to C-terminus (i) an optional leader peptide, (ii) the first CAR, (iii) a linker, and (iv) an invariant TCRα chain, and wherein the second construct comprises in the order of N-terminus to C-terminus (i) an optional leader peptide, (ii) the second CAR, (iii) a linker, and (iv) an invariant TCRβ chain.

4. (canceled)

5. The antigen binding system of claim 3, wherein the linker is a cleavable linker.

6. The antigen binding system of claim 5, wherein the cleavable linker is a P2A or T2A linker.

7. The antigen binding system of claim 3, wherein the linker is a linker according to SEQ ID NOS: 247, 293, 294, or 296-300.

8. (canceled)

9. The antigen binding system of claim 1, wherein the first CAR comprises a first binding motif that binds a first antigen and the second CAR comprises a second binding motif that binds a second antigen.

10. The antigen binding system of claim 1, wherein the first CAR and the second CAR both further comprise (i) a hinge, (ii) a transmembrane domain, and (iii) an intracellular domain comprising a costimulatory domain and an activation domain.

11. (canceled)

12. The antigen binding system of claim 9, wherein the first antigen and the second antigen are selected from the group consisting of 5T4, alphafetoprotein, B cell maturation antigen (BCMA), TACI, CA-125, carcinoembryonic antigen, CD19, CD20, CD22, CD23, CD30, CD33, CD56, CD123, CD138, c-Met, CSPG4, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gp120, IL-11Ralpha, kappa chain, lambda chain, melanoma-associated antigen, mesothelin, MUC-1, mutated p53, mutated ras, prostate-specific antigen, ROR1, VEGFR2, and wherein the first antigen and the second antigen are different.

13. The antigen binding system of claim 9, wherein the first binding motif is a CD19 or a CD20 binding motif and the second binding motif is a CD19 or a CD20 binding motif, and wherein the first binding motif and the second binding motif are different.

14. The antigen binding system of claim 13, wherein the CD19 binding motif comprises a first domain comprising three heavy chain complementarity determining regions (CDRH1, CDRH2, and CDRH3) and a second domain comprising three light chain complementarity determining regions (CDRL1, CDRL2, and CDRL3), wherein
(i) the CDRH1 has a sequence according to any one of SEQ ID NOs: 223-225;
(ii) the CDRH2 has a sequence according to any one of SEQ ID NOs: 226-228;
(iii) the CDRH3 has a sequence according to any one of SEQ ID NOs: 229-231;
(iv) the CDRL1 has a sequence according to any one of SEQ ID NOs: 234-236;
(v) the CDRL2 has a sequence according to any one of SEQ ID NOs: 237-239; and
(vi) the CDRL3 has a sequence according to any one of SEQ ID NOs: 240-242; and
wherein the CD20 binding motif comprises a first domain comprising three heavy chain complementarity determining regions (CDRH1, CDRH2, and CDRH) and a second domain comprising three light chain complementarity determining regions (CDRL1, CDRL2, and CDRL3), wherein
(i) the CDRH1 has a sequence according to any one of SEQ ID NOs: 3-5, 25-27, 47-49, 69-71, 91-93, 113-115, 135-137, 157-159, 179-181, and 201-203;
(ii) the CDRH2 has a sequence according to any one of SEQ ID NOs: 6-8, 28-30, 50-52, 72-74, 94-96, 116-118, 138-140, 160-162, 182-184, and 204-206;
(iii) the CDRH3 has a sequence according to any one of SEQ ID NOs: 9-11, 31-33, 53-55, 75-77, 96-98, 119-121, 141-143, 163-165, 185-187, and 207-209;
(iv) the CDRL1 has a sequence according to any one of SEQ ID NOs: 14-16, 36-38, 58-60, 80-82, 102-104, 124-126, 146-148, 168-170, 190-192, and 212-214;
(v) the CDRL2 has a sequence according to any one of SEQ ID NOs: 17-19, 39-41, 61-63, 83-85, 105-107, 127-129, 149-151, 171-173, 193-195, and 215-217; and
(vi) the CDRL3 has a sequence according to any one of SEQ ID NOs: 20-22, 42-44, 64-66, 86-88, 108-110, 130-132, 152-154, 174-176, 196-198, and 218-220.

15. (canceled)

16. The antigen binding system of claim 13, wherein the CD19 binding motif comprises a heavy chain variable domain comprising the three CDRHs and a light chain variable domain comprising the three CDRLs, wherein:
(i) the heavy chain variable domain is at least 80% identical to SEQ ID NO: 221; and
(ii) the light chain variable domain is at least 80% identical to SEQ ID NO: 233; and
wherein the CD20 binding motif comprises a heavy chain variable domain comprising the three CDRHs and a light chain variable domain comprising the three CDRLs, wherein:
(i) the heavy chain variable domain is at least 80% identical to SEQ ID NOs: 1, 23, 45, 67, 89, 111, 133, 155, 177, or 199; and
(ii) the light chain variable domain is at least 80% identical to SEQ ID NOs: 12, 34, 56, 78, 100, 122, 144, 166, 188, or 210.

17. The antigen binding system of claim 13, wherein the CD19 binding motif comprises a first heavy chain variable domain comprising the three CDRHs and a light chain variable domain comprising the three CDRLs, wherein:
(i) the heavy chain variable domain is at least 80% identical to SEQ ID NO: 221 and the light chain variable domain is at least 80% identical to SEQ ID NO: 233; and wherein the CD20 binding motif comprises a first heavy chain variable domain comprising the three CDRHs and a light chain variable domain comprising the three CDRLs, wherein:
(i) the heavy chain variable domain is at least 80% identical to SEQ ID NO: 1 and the light chain variable domain is at least 80% identical to SEQ ID NO: 12;
(ii) the heavy chain variable domain is at least 80% identical to SEQ ID NO: 23 and the light chain variable domain is at least 80% identical to SEQ ID NO: 34;
(iii) the heavy chain variable domain is at least 80% identical to SEQ ID NO: 45 and the light chain variable domain is at least 80% identical to SEQ ID NO: 56;
(iv) the heavy chain variable domain is at least 80% identical to SEQ ID NO: 67 and the light chain variable domain is at least 80% identical to SEQ ID NO: 78;
(v) the heavy chain variable domain is at least 80% identical to SEQ ID NO: 89 and the light chain variable domain is at least 80% identical to SEQ ID NO: 100;
(vi) the heavy chain variable domain is at least 80% identical to SEQ ID NO: 111 and the light chain variable domain is at least 80% identical to SEQ ID NO: 122;
(vii) the heavy chain variable domain is at least 80% identical to SEQ ID NO: 133 and the light chain variable domain is at least 80% identical to SEQ ID NO: 144;
(viii) the heavy chain variable domain is at least 80% identical to SEQ ID NO: 155 and the light chain variable domain is at least 80% identical to SEQ ID NO: 166.
(ix) the heavy chain variable domain is at least 80% identical to SEQ ID NO: 177 and the light chain variable domain is at least 80% identical to SEQ ID NO: 188; or
(x) the heavy chain variable domain is at least 80% identical to SEQ ID NO: 199 and the light chain variable domain is at least 80% identical to SEQ ID NO: 210.

18. The antigen binding system of claim 1, wherein the antigen binding system further comprises (i) a first vector comprising a nucleic acid encoding the first construct and (ii) a second vector comprising a nucleic acid encoding the second construct.

19. A nucleic acid encoding the antigen binding system of claim 1.

20. A vector comprising the nucleic acid of claim 19.

21-22. (canceled)

23. A cell that comprises the antigen binding system of claim 1.

24. (canceled)

25. A pharmaceutical composition comprising the cell of claim 23.

26. A method of generating an engineered cell, the method comprising transfecting or transducing a cell with the vector of claim 20.

27. The method of claim 26, wherein the engineered cell is an autologous cell or an allogeneic cell.

28. (canceled)


SEQUENCE LISTING (302 sequences)

SEQ ID NO: 1 - protein, 120 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARGGGSWYSNWFDPWGQGTMVTVSS

SEQ ID NO: 2 - DNA, 360 bp, synthetic construct
  source 1..360: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcgaccatagtggaagcaccaactacaacccgtccctcaagagtcgagtcaccatatcagtagacacgtccaagaaccagttctccctgaagctgagctctgtgaccgccgcggacacggcggtgtactactgcgccagaggtggaggaagttggtacagcaactggttcgacccatggggacagggtacaatggtcaccgtctcctca

SEQ ID NO: 3 - protein, 8 aa, synthetic construct
GGSFSGYY

SEQ ID NO: 4 - protein, 5 aa, synthetic construct
GYYWS

SEQ ID NO: 5 - protein, 6 aa, synthetic construct
GGSFSG

SEQ ID NO: 6 - protein, 7 aa, synthetic construct
IDHSGST

SEQ ID NO: 7 - protein, 16 aa, synthetic construct
EIDHSGSTNYNPSLKS

SEQ ID NO: 8 - protein, 5 aa, synthetic construct
DHSGS

SEQ ID NO: 9 - protein, 14 aa, synthetic construct
ARGGGSWYSNWFDP

SEQ ID NO: 10 - protein, 12 aa, synthetic construct
GGGSWYSNWFDP

SEQ ID NO: 11 - protein, 12 aa, synthetic construct
GGGSWYSNWFDP

SEQ ID NO: 12 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQDRSLPPTFGGGTKVEIK

SEQ ID NO: 13 - DNA, 321 bp, synthetic construct
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgccagcaggaccgaagtctccctcctacttttggcggagggaccaaggttgagatcaaa

SEQ ID NO: 14 - protein, 11 aa, synthetic construct
RASQSISSWLA

SEQ ID NO: 15 - protein, 11 aa, synthetic construct
RASQSISSWLA

SEQ ID NO: 16 - protein, 11 aa, synthetic construct
RASQSISSWLA

SEQ ID NO: 17 - protein, 7 aa, synthetic construct
DASSLES

SEQ ID NO: 18 - protein, 7 aa, synthetic construct
DASSLES

SEQ ID NO: 19 - protein, 7 aa, synthetic construct
DASSLES

SEQ ID NO: 20 - protein, 9 aa, synthetic construct
QQDRSLPPT

SEQ ID NO: 21 - protein, 9 aa, synthetic construct
QQDRSLPPT

SEQ ID NO: 22 - protein, 9 aa, synthetic construct
QQDRSLPPT

SEQ ID NO: 23 - protein, 121 aa, synthetic construct
QVQLQQWGAGLLKPSETLSLTCAVYGGSFSGIHWNWIRQPPGKGLEWIGDIDTSGSTNYNPSLKSRVTISVDTSKNQFSLKLSSVTAADTAVYYCARLGQESATYLGMDVWGQGTTVTVSS

SEQ ID NO: 24 - DNA, 363 bp, synthetic construct
  source 1..363: mol_type = other DNA
caggtgcagctacagcagtggggcgcaggactgttgaagccttcggagaccctgtccctcacctgcgctgtctatggtgggtccttcagtggtatccactggaactggatccgccagcccccagggaaggggctggagtggattggggacatcgacacaagtggaagcaccaactacaacccgtccctcaagagtcgagtcaccatatccgtagacacgtccaagaaccagttctccctgaagctgagttctgtgaccgccgcagacacggcggtgtactactgcgccagattgggacaggagtcagccacctatctcggaatggacgtatggggccagggaacaactgtcaccgtctcctca

SEQ ID NO: 25 - protein, 8 aa, synthetic construct
GGSFSGIH

SEQ ID NO: 26 - protein, 5 aa, synthetic construct
GIHWN

SEQ ID NO: 27 - protein, 6 aa, synthetic construct
GGSFSG

SEQ ID NO: 28 - protein, 7 aa, synthetic construct
IDTSGST

SEQ ID NO: 29 - protein, 16 aa, synthetic construct
DIDTSGSTNYNPSLKS

SEQ ID NO: 30 - protein, 5 aa, synthetic construct
DTSGS

SEQ ID NO: 31 - protein, 15 aa, synthetic construct
ARLGQESATYLGMDV

SEQ ID NO: 32 - protein, 13 aa, synthetic construct
LGQESATYLGMDV

SEQ ID NO: 33 - protein, 13 aa, synthetic construct
LGQESATYLGMDV

SEQ ID NO: 34 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQLYTYPFTFGGGTKVEIK

SEQ ID NO: 35 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagctctacacctaccctttcacttttggcggagggaccaaggttgagatcaaa

SEQ ID NO: 36 - protein, 17 aa, synthetic construct
KSSQSVLYSSNNKNYLA

SEQ ID NO: 37 - protein, 17 aa, synthetic construct
KSSQSVLYSSNNKNYLA

SEQ ID NO: 38 - protein, 17 aa, synthetic construct
KSSQSVLYSSNNKNYLA

SEQ ID NO: 39 - protein, 7 aa, synthetic construct
WASTRES

SEQ ID NO: 40 - protein, 7 aa, synthetic construct
WASTRES

SEQ ID NO: 41 - protein, 7 aa, synthetic construct
WASTRES

SEQ ID NO: 42 - protein, 9 aa, synthetic construct
QQLYTYPFT

SEQ ID NO: 43 - protein, 9 aa, synthetic construct
QQLYTYPFT

SEQ ID NO: 44 - protein, 9 aa, synthetic construct
QQLYTYPFT

SEQ ID NO: 45 - protein, 124 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSS

SEQ ID NO: 46 - DNA, 372 bp, synthetic construct
  source 1..372: mol_type = other DNA
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagttctgtgaccgccgcagacacggcggtgtactactgcgccagagagactgactacagcagcggaatgggatacggaatggacgtatggggccagggaacaactgtcaccgtctcctca

SEQ ID NO: 47 - protein, 10 aa, synthetic construct
GGSISSSSYY

SEQ ID NO: 48 - protein, 7 aa, synthetic construct
SSSYYWG

SEQ ID NO: 49 - protein, 8 aa, synthetic construct
GGSISSSS

SEQ ID NO: 50 - protein, 7 aa, synthetic construct
IYYSGST

SEQ ID NO: 51 - protein, 16 aa, synthetic construct
SIYYSGSTYYNPSLKS

SEQ ID NO: 52 - protein, 5 aa, synthetic construct
YYSGS

SEQ ID NO: 53 - protein, 16 aa, synthetic construct
ARETDYSSGMGYGMDV

SEQ ID NO: 54 - protein, 14 aa, synthetic construct
ETDYSSGMGYGMDV

SEQ ID NO: 55 - protein, 14 aa, synthetic construct
ETDYSSGMGYGMDV

SEQ ID NO: 56 - protein, 107 aa, synthetic construct
DIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIK

SEQ ID NO: 57 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattaacagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgccagcaaagcctcgccgaccctttcacttttggcggagggaccaaggttgagatcaaa

SEQ ID NO: 58 - protein, 11 aa, synthetic construct
RASQSINSYLN

SEQ ID NO: 59 - protein, 11 aa, synthetic construct
RASQSINSYLN

SEQ ID NO: 60 - protein, 11 aa, synthetic construct
RASQSINSYLN

SEQ ID NO: 61 - protein, 7 aa, synthetic construct
AASSLQS

SEQ ID NO: 62 - protein, 7 aa, synthetic construct
AASSLQS

SEQ ID NO: 63 - protein, 7 aa, synthetic construct
AASSLQS

SEQ ID NO: 64 - protein, 9 aa, synthetic construct
QQSLADPFT

SEQ ID NO: 65 - protein, 9 aa, synthetic construct
QQSLADPFT

SEQ ID NO: 66 - protein, 9 aa, synthetic construct
QQSLADPFT

SEQ ID NO: 67 - protein, 125 aa, synthetic construct
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSS

SEQ ID NO: 68 - DNA, 375 bp, synthetic construct
  source 1..375: mol_type = other DNA
caggttcagctggtgcagtctggagctgaggtgaagaagcctggggcctcagtgaaggtctcctgcaaggcttctggttacacctttaaagaatatggtatcagctgggtgcgacaggcccctggacaagggcttgagtggatgggatggatcagcgcttacagtggtcacacatactatgcacagaagctccagggcagagtcaccatgaccacagacacatccacgagcacagcctacatggagctgaggagcctgagatctgacgacacggcggtgtactactgcgccagagggcctcactacgacgactggagcggatttatcatatggttcgacccatggggacagggtacattggtcaccgtctcctca

SEQ ID NO: 69 - protein, 8 aa, synthetic construct
GYTFKEYG

SEQ ID NO: 70 - protein, 5 aa, synthetic construct
EYGIS

SEQ ID NO: 71 - protein, 6 aa, synthetic construct
GYTFKE

SEQ ID NO: 72 - protein, 8 aa, synthetic construct
ISAYSGHT

SEQ ID NO: 73 - protein, 16 aa, synthetic construct
WISAYSGHTYYAQKLQ

SEQ ID NO: 74 - protein, 5 aa, synthetic construct
SAYSG

SEQ ID NO: 75 - protein, 18 aa, synthetic construct
ARGPHYDDWSGFIIWFDP

SEQ ID NO: 76 - protein, 16 aa, synthetic construct
GPHYDDWSGFIIWFDP

SEQ ID NO: 77 - protein, 16 aa, synthetic construct
GPHYDDWSGFIIWFDP

SEQ ID NO: 78 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIK

SEQ ID NO: 79 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccttcaaggttcagtggcagtggatctgggacagatttcactctcaccatcagcagtctgcaacctgaagattttgcaacttactactgtcaacagagttacaggtttcctcctacctttggccaagggaccaaggttgagatcaaa

SEQ ID NO: 80 - protein, 11 aa, synthetic construct
RASQSISSYLN

SEQ ID NO: 81 - protein, 11 aa, synthetic construct
RASQSISSYLN

SEQ ID NO: 82 - protein, 11 aa, synthetic construct
RASQSISSYLN

SEQ ID NO: 83 - protein, 7 aa, synthetic construct
AASSLQS

SEQ ID NO: 84 - protein, 7 aa, synthetic construct
AASSLQS

SEQ ID NO: 85 - protein, 7 aa, synthetic construct
AASSLQS

SEQ ID NO: 86 - protein, 9 aa, synthetic construct
QQSYRFPPT

SEQ ID NO: 87 - protein, 9 aa, synthetic construct
QQSYRFPPT

SEQ ID NO: 88 - protein, 9 aa, synthetic construct
QQSYRFPPT

SEQ ID NO: 89 - protein, 124 aa, synthetic construct
QVQLQESGPGLVKPSETLSLTCTVSGGSISSPDHYWGWIRQPPGKGLEWIGSIYASGSTFYNPSLKSRVTISVDTSKNQFSLKLSSVTAADTAVYYCARETDYSSGMGYGMDVWGQGTTVTVSS

SEQ ID NO: 90 - DNA, 372 bp, synthetic construct
  source 1..372: mol_type = other DNA
caggtgcagctgcaggagtcgggcccaggactggtgaagccttcggagaccctgtccctcacctgcactgtctctggtggctccatcagcagtcccgaccactactggggctggatccgccagcccccagggaaggggctggagtggattgggtccatctacgccagtgggagcaccttctacaacccgtccctcaagagtcgagtcaccatatccgtagacacgtccaagaaccagttctccctgaagctgagctctgtgaccgccgcggacacggcggtgtactactgcgccagagagactgactacagcagcggaatgggatacggaatggacgtatggggccagggaacaactgtcaccgtctcctca

SEQ ID NO: 91 - protein, 10 aa, synthetic construct
GGSISSPDHY

SEQ ID NO: 92 - protein, 7 aa, synthetic construct
SPDHYWG

SEQ ID NO: 93 - protein, 8 aa, synthetic construct
GGSISSPD

SEQ ID NO: 94 - protein, 7 aa, synthetic construct
IYASGST

SEQ ID NO: 95 - protein, 16 aa, synthetic construct
SIYASGSTFYNPSLKS

SEQ ID NO: 96 - protein, 5 aa, synthetic construct
YASGS

SEQ ID NO: 97 - protein, 16 aa, synthetic construct
ARETDYSSGMGYGMDV

SEQ ID NO: 98 - protein, 14 aa, synthetic construct
ETDYSSGMGYGMDV

SEQ ID NO: 99 - protein, 14 aa, synthetic construct
ETDYSSGMGYGMDV

SEQ ID NO: 100 - protein, 107 aa, synthetic construct; identical to SEQ ID NO: 56

SEQ ID NO: 101 - DNA, 321 bp, synthetic construct; identical to SEQ ID NO: 57
  source 1..321: mol_type = other DNA

SEQ ID NO: 102 - protein, 11 aa, synthetic construct
RASQSINSYLN

SEQ ID NO: 103 - protein, 11 aa, synthetic construct
RASQSINSYLN

SEQ ID NO: 104 - protein, 11 aa, synthetic construct
RASQSINSYLN

SEQ ID NO: 105 - protein, 7 aa, synthetic construct
AASSLQS

SEQ ID NO: 106 - protein, 7 aa, synthetic construct
AASSLQS

SEQ ID NO: 107 - protein, 7 aa, synthetic construct
AASSLQS

SEQ ID NO: 108 - protein, 9 aa, synthetic construct
QQSLADPFT

SEQ ID NO: 109 - protein, 9 aa, synthetic construct
QQSLADPFT

SEQ ID NO: 110 - protein, 9 aa, synthetic construct
QQSLADPFT

SEQ ID NO: 111 - protein, 123 aa, synthetic construct
QITLKESGPTLVKPTQTLTLTCTFSGFSLDTEGVGVGWIRQPPGKALEWLALIYFNDQKRYSPSLKSRLTITKDTSKNQVVLTMTNMDPVDTAVYYCARDTGYSRWYYGMDVWGQGTTVTVSS

SEQ ID NO: 112 - DNA, 369 bp, synthetic construct
  source 1..369: mol_type = other DNA
cagatcaccttgaaggagtctggtcctacgctggtgaaacccacacagaccctcacgctgacctgcaccttctctgggttctcactcgacactgaaggagtgggtgtgggctggatccgtcagcccccaggaaaggccctggagtggcttgcactcatttatttcaatgatcaaaagcgctacagcccatctctgaagagcaggctcaccatcaccaaggacacctccaaaaaccaggtggtccttacaatgaccaacatggaccctgtggacacggcggtgtactactgcgccagagacacgggatacagccgatggtactacggcatggatgtatggggccagggaacaactgtcaccgtctcctca

SEQ ID NO: 113 - protein, 10 aa, synthetic construct
GFSLDTEGVG

SEQ ID NO: 114 - protein, 7 aa, synthetic construct
TEGVGVG

SEQ ID NO: 115 - protein, 8 aa, synthetic construct
GFSLDTEG

SEQ ID NO: 116 - protein, 7 aa, synthetic construct
IYFNDQK

SEQ ID NO: 117 - protein, 16 aa, synthetic construct
LIYFNDQKRYSPSLKS

SEQ ID NO: 118 - protein, 5 aa, synthetic construct
YFNDQ

SEQ ID NO: 119 - protein, 15 aa, synthetic construct
ARDTGYSRWYYGMDV

SEQ ID NO: 120 - protein, 13 aa, synthetic construct
DTGYSRWYYGMDV

SEQ ID NO: 121 - protein, 13 aa, synthetic construct
DTGYSRWYYGMDV

SEQ ID NO: 122 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQAYAYPITFGGGTKVEIK

SEQ ID NO: 123 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcagcaggcatacgcctaccctatcacttttggcggagggaccaaggttgagatcaaa

SEQ ID NO: 124 - protein, 11 aa, synthetic construct
RASQGISSWLA

SEQ ID NO: 125 - protein, 11 aa, synthetic construct
RASQGISSWLA

SEQ ID NO: 126 - protein, 11 aa, synthetic construct
RASQGISSWLA

SEQ ID NO: 127 - protein, 7 aa, synthetic construct
AASSLQS

SEQ ID NO: 128 - protein, 7 aa, synthetic construct
AASSLQS

SEQ ID NO: 129 - protein, 7 aa, synthetic construct
AASSLQS

SEQ ID NO: 130 - protein, 9 aa, synthetic construct
QQAYAYPIT

SEQ ID NO: 131 - protein, 9 aa, synthetic construct
QQAYAYPIT

SEQ ID NO: 132 - protein, 9 aa, synthetic construct
QQAYAYPIT

SEQ ID NO: 133 - protein, 127 aa, synthetic construct
QVQLQQWGAGLLKPSETLSLTCAVYGGSFEKYYWSWIRQPPGKGLEWIGEIYHSGLTNYNPSLKSRVTISVDTSKNQFSLKLSSVTAADTAVYYCARVRYDSSDSYYYSYDYGMDVWGQGTTVTVSS

SEQ ID NO: 134 - DNA, 381 bp, synthetic construct
  source 1..381: mol_type = other DNA
caggtgcagctacagcagtggggcgcaggactgttgaagccttcggagaccctgtccctcacctgcgctgtctatggtgggtccttcgaaaaatactactggagctggatccgccagcccccagggaaggggctggagtggattggggaaatctaccatagtggactcaccaactacaacccgtccctcaagagtcgagtcaccatatcagtagacacgtccaagaaccagttctccctgaagctgagctctgtgaccgccgcggacacggcggtgtactactgcgccagggtcagatacgacagcagcgactcctactactatagctacgattatggaatggacgtatggggccagggaacaactgtcaccgtctcctca

SEQ ID NO: 135 - protein, 8 aa, synthetic construct
GGSFEKYY

SEQ ID NO: 136 - protein, 5 aa, synthetic construct
KYYWS

SEQ ID NO: 137 - protein, 6 aa, synthetic construct
GGSFEK

SEQ ID NO: 138 - protein, 7 aa, synthetic construct
IYHSGLT

SEQ ID NO: 139 - protein, 16 aa, synthetic construct
EIYHSGLTNYNPSLKS

SEQ ID NO: 140 - protein, 5 aa, synthetic construct
YHSGL

SEQ ID NO: 141 - protein, 21 aa, synthetic construct
ARVRYDSSDSYYYSYDYGMDV

SEQ ID NO: 142 - protein, 19 aa, synthetic construct
VRYDSSDSYYYSYDYGMDV

SEQ ID NO: 143 - protein, 19 aa, synthetic construct
VRYDSSDSYYYSYDYGMDV

SEQ ID NO: 144 - protein, 113 aa, synthetic construct
DIVLTQSPDSLAVSLGERATINCKSSQSVLYSSNNKNYLAWYQQKPGQPPKLLIYWASSRESGVPDRFSGSGSGTDFTLTISSLQAEDVAVYYCQQSYSFPWTFGGGTKVEIK

SEQ ID NO: 145 - DNA, 339 bp, synthetic construct
  source 1..339: mol_type = other DNA
gacatcgtgctgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctagccgggaatccggggtccctgaccgattcagtggcagcgggtctgggacagatttcactctcaccatcagcagcctgcaggctgaagatgtggcagtttattactgtcagcagtcctactccttcccttggacttttggcggagggaccaaggttgagatcaaa

SEQ ID NO: 146 - protein, 17 aa, synthetic construct
KSSQSVLYSSNNKNYLA

SEQ ID NO: 147 - protein, 17 aa, synthetic construct
KSSQSVLYSSNNKNYLA

SEQ ID NO: 148 - protein, 17 aa, synthetic construct
KSSQSVLYSSNNKNYLA

SEQ ID NO: 149 - protein, 7 aa, synthetic construct
WASSRES

SEQ ID NO: 150 - protein, 7 aa, synthetic construct
WASSRES

SEQ ID NO: 151 - protein, 7 aa, synthetic construct
WASSRES

SEQ ID NO: 152 - protein, 9 aa, synthetic construct
QQSYSFPWT

SEQ ID NO: 153 - protein, 9 aa, synthetic construct
QQSYSFPWT

SEQ ID NO: 154 - protein, 9 aa, synthetic construct
QQSYSFPWT

SEQ ID NO: 155 - protein, 127 aa, synthetic construct
QVQLQQWGAGLLKPSETLSLTCAVYGGSFSRYVWSWIRQPPGKGLEWIGEIDSSGKTNYNPSLKSRVTISVDTSKNQFSLKLSSVTAADTAVYYCARVRYDSSDSYYYSYDYGMDVWGQGTTVTVSS

SEQ ID NO: 156 - DNA, 381 bp, synthetic construct
  source 1..381: mol_type = other DNA
caggtgcagctacagcagtggggcgcaggactgttgaagccttcggagaccctgtccctcacctgcgctgtctatggtgggtccttcagtcgatacgtatggagctggatccgccagcccccagggaaggggctggagtggattggggaaatcgactccagtggaaaaaccaactacaacccgtccctcaagagtcgagtcaccatatcagtagacacgtccaagaaccagttctccctgaagctgagctctgtgaccgccgcggacacggcggtgtactactgcgccagggtcagatacgacagcagcgactcctactactatagctacgattatggaatggacgtatggggccagggaacaactgtcaccgtctcctca

SEQ ID NO: 157 - protein, 8 aa, synthetic construct
GGSFSRYV

SEQ ID NO: 158 - protein, 5 aa, synthetic construct
RYVWS

SEQ ID NO: 159 - protein, 6 aa, synthetic construct
GGSFSR

SEQ ID NO: 160 - protein, 7 aa, synthetic construct
IDSSGKT

SEQ ID NO: 161 - protein, 16 aa, synthetic construct
EIDSSGKTNYNPSLKS

SEQ ID NO: 162 - protein, 5 aa, synthetic construct
DSSGK

SEQ ID NO: 163 - protein, 21 aa, synthetic construct
ARVRYDSSDSYYYSYDYGMDV

SEQ ID NO: 164 - protein, 19 aa, synthetic construct
VRYDSSDSYYYSYDYGMDV

SEQ ID NO: 165 - protein, 19 aa, synthetic construct
VRYDSSDSYYYSYDYGMDV

SEQ ID NO: 166 - protein, 113 aa, synthetic construct; identical to SEQ ID NO: 144

SEQ ID NO: 167 - DNA, 339 bp, synthetic construct; identical to SEQ ID NO: 145
  source 1..339: mol_type = other DNA

SEQ ID NO: 168 - protein, 17 aa, synthetic construct
KSSQSVLYSSNNKNYLA

SEQ ID NO: 169 - protein, 17 aa, synthetic construct
KSSQSVLYSSNNKNYLA

SEQ ID NO: 170 - protein, 17 aa, synthetic construct
KSSQSVLYSSNNKNYLA

SEQ ID NO: 171 - protein, 7 aa, synthetic construct
WASSRES

SEQ ID NO: 172 - protein, 7 aa, synthetic construct
WASSRES

SEQ ID NO: 173 - protein, 7 aa, synthetic construct
WASSRES

SEQ ID NO: 174 - protein, 9 aa, synthetic construct
QQSYSFPWT

SEQ ID NO: 175 - protein, 9 aa, synthetic construct
QQSYSFPWT

SEQ ID NO: 176 - protein, 9 aa, synthetic construct
QQSYSFPWT

SEQ ID NO: 177 - protein, 127 aa, synthetic construct
QVQLQQWGAGLLKPSETLSLTCAVYGGSFSGYAWSWIRQPPGKGLEWIGEIDHRGFTNYNPSLKSRVTISVDTSKNQFSLKLSSVTAADTAVYYCARVRYDSSDSYYYSYDYGMDVWGQGTTVTVSS

SEQ ID NO: 178 - DNA, 381 bp, synthetic construct
  source 1..381: mol_type = other DNA
caggtgcagctacagcagtggggcgcaggactgttgaagccttcggagaccctgtccctcacctgcgctgtctatggtgggtccttctccggttacgcatggagctggatccgccagcccccagggaaggggctggagtggattggggaaatcgaccatcgaggattcaccaactacaacccgtccctcaagagtcgagtcaccatatcagtagacacgtccaagaaccagttctccctgaagctgagctctgtgaccgccgcggacacggcggtgtactactgcgccagggtcagatacgacagcagcgactcctactactatagctacgattatggaatggacgtatggggccagggaacaactgtcaccgtctcctca

SEQ ID NO: 179 - protein, 8 aa, synthetic construct
GGSFSGYA

SEQ ID NO: 180 - protein, 5 aa, synthetic construct
GYAWS

SEQ ID NO: 181 - protein, 6 aa, synthetic construct
GGSFSG

SEQ ID NO: 182 - protein, 7 aa, synthetic construct
IDHRGFT

SEQ ID NO: 183 - protein, 16 aa, synthetic construct
EIDHRGFTNYNPSLKS

SEQ ID NO: 184 - protein, 5 aa, synthetic construct
DHRGF

SEQ ID NO: 185 - protein, 21 aa, synthetic construct
ARVRYDSSDSYYYSYDYGMDV

SEQ ID NO: 186 - protein, 19 aa, synthetic construct
VRYDSSDSYYYSYDYGMDV

SEQ ID NO: 187 - protein, 19 aa, synthetic construct
VRYDSSDSYYYSYDYGMDV

SEQ ID NO: 188 - protein, 113 aa, synthetic construct; identical to SEQ ID NO: 144

SEQ ID NO: 189 - DNA, 339 bp, synthetic construct; identical to SEQ ID NO: 145
  source 1..339: mol_type = other DNA

SEQ ID NO: 190 - protein, 17 aa, synthetic construct
KSSQSVLYSSNNKNYLA

SEQ ID NO: 191 - protein, 17 aa, synthetic construct
KSSQSVLYSSNNKNYLA

SEQ ID NO: 192 - protein, 17 aa, synthetic construct
KSSQSVLYSSNNKNYLA

SEQ ID NO: 193 - protein, 7 aa, synthetic construct
WASSRES

SEQ ID NO: 194 - protein, 7 aa, synthetic construct
WASSRES

SEQ ID NO: 195 - protein, 7 aa, synthetic construct
WASSRES

SEQ ID NO: 196 - protein, 9 aa, synthetic construct
QQSYSFPWT

SEQ ID NO: 197 - protein, 9 aa, synthetic construct
QQSYSFPWT

SEQ ID NO: 198 - protein, 9 aa, synthetic construct
QQSYSFPWT

SEQ ID NO: 199 - protein, 123 aa, synthetic construct
QVQLQQWGAGLLKPSETLSLTCAVYGGSFQKYYWSWIRQPPGKGLEWIGEIDTSGFTNYNPSLKSRVTISVDTSKNQFSLKLSSVTAADTAVYYCARVGRYSYGYYITAFDIWGQGTTVTVSS

SEQ ID NO: 200 - DNA, 369 bp, synthetic construct
  source 1..369: mol_type = other DNA
caggtgcagctacagcagtggggcgcaggactgttgaagccttcggagaccctgtccctcacctgcgctgtctatggtgggtccttccaaaaatactactggagctggatccgccagcccccagggaaggggctggagtggattggggaaatcgacaccagtggattcaccaactacaacccgtccctcaagagtcgagtcaccatatcagtagacacgtccaagaaccagttctccctgaagctgagctctgtgaccgccgcggacacggcggtgtactactgcgccagagtgggaaggtacagctacggatactatatcaccgcattcgacatatggggtcagggtacaactgtcaccgtctcctca

SEQ ID NO: 201 - protein, 8 aa, synthetic construct
GGSFQKYY

SEQ ID NO: 202 - protein, 5 aa, synthetic construct
KYYWS

SEQ ID NO: 203 - protein, 6 aa, synthetic construct
GGSFQK

SEQ ID NO: 204 - protein, 7 aa, synthetic construct
IDTSGFT

SEQ ID NO: 205 - protein, 16 aa, synthetic construct
EIDTSGFTNYNPSLKS

SEQ ID NO: 206 - protein, 5 aa, synthetic construct
DTSGF

SEQ ID NO: 207 - protein, 17 aa, synthetic construct
ARVGRYSYGYYITAFDI

SEQ ID NO: 208 - protein, 15 aa, synthetic construct
VGRYSYGYYITAFDI

SEQ ID NO: 209 - protein, 15 aa, synthetic construct
VGRYSYGYYITAFDI

SEQ ID NO: 210 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQHYSFPFTFGGGTKVEIK

SEQ ID NO: 211 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagcactactccttccctttcacttttggcggagggaccaaggttgagatcaaa

SEQ ID NO: 212 - protein, 17 aa, synthetic construct
KSSQSVLYSSNNKNYLA

SEQ ID NO: 213 - protein, 17 aa, synthetic construct
KSSQSVLYSSNNKNYLA

SEQ ID NO: 214 - protein, 17 aa, synthetic construct
KSSQSVLYSSNNKNYLA

SEQ ID NO: 215 - protein, 7 aa, synthetic construct
WASTRES

SEQ ID NO: 216 - protein, 7 aa, synthetic construct
WASTRES

SEQ ID NO: 217 - protein, 7 aa, synthetic construct
WASTRES

SEQ ID NO: 218 - protein, 9 aa, synthetic construct
QQHYSFPFT

SEQ ID NO: 219 - protein, 9 aa, synthetic construct
QQHYSFPFT

SEQ ID NO: 220 - protein, 9 aa, synthetic construct
QQHYSFPFT

SEQ ID NO: 221 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 222 - DNA, 360 bp, synthetic construct
  source 1..360: mol_type = other DNA
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 223 - protein, 8 aa, synthetic construct
GVSLPDYG

SEQ ID NO: 224 - protein, 5 aa, synthetic construct
DYGVS

SEQ ID NO: 225 - protein, 7 aa, synthetic construct
GVSLPDY

SEQ ID NO: 226 - protein, 7 aa, synthetic construct
IWGSETT

SEQ ID NO: 227 - protein, 16 aa, synthetic construct
VIWGSETTYYNSALKS

SEQ ID NO: 228 - protein, 5 aa, synthetic construct
WGSET

SEQ ID NO: 229 - protein, 14 aa, synthetic construct
AKHYYYGGSYAMDY

SEQ ID NO: 230 - protein, 12 aa, synthetic construct
HYYYGGSYAMDY

SEQ ID NO: 231 - protein, 12 aa, synthetic construct
HYYYGGSYAMDY

SEQ ID NO: 232 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 233 - DNA, 321 bp, synthetic construct
  source 1..321: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 234 - protein, 11 aa, synthetic construct
RASQDISKYLN

SEQ ID NO: 235 - protein, 11 aa, synthetic construct
RASQDISKYLN

SEQ ID NO: 236 - protein, 11 aa, synthetic construct
RASQDISKYLN

SEQ ID NO: 237 - protein, 7 aa, synthetic construct
HTSRLHS

SEQ ID NO: 238 - protein, 7 aa, synthetic construct
HTSRLHS

SEQ ID NO: 239 - protein, 7 aa, synthetic construct
HTSRLHS

SEQ ID NO: 240 - protein, 9 aa, synthetic construct
QQGNTLPYT

SEQ ID NO: 241 - protein, 9 aa, synthetic construct
QQGNTLPYT

SEQ ID NO: 242 - protein, 9 aa, synthetic construct
QQGNTLPYT

SEQ ID NO: 243 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 244 - DNA, 735 bp, synthetic construct
  source 1..735: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 245 - protein, 22 aa, synthetic construct
MLLLVTSLLLCELPHPAFLLIP

SEQ ID NO: 246 - DNA, 66 bp, synthetic construct
  source 1..66: mol_type = other DNA
atgcttctcctggtgacaagccttctgctctgtgagttaccacacccagcattcctcctgatccca

SEQ ID NO: 247 - protein, 18 aa, synthetic construct
GSTSGSGKPGSGEGSTKG

SEQ ID NO: 248 - DNA, 54 bp, synthetic construct
  source 1..54: mol_type = other DNA
ggctccacctctggatccggcaagcccggatctggcgagggatccaccaagggc

SEQ ID NO: 249 - protein, 19 aa, synthetic construct
MEWTWVFLFLLSVTAGVHS

SEQ ID NO: 250 - protein, 21 aa, synthetic construct
MALPVTALLLPLALLLHAARP

SEQ ID NO: 251 - DNA, 813 bp, synthetic construct
  source 1..813: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcagcggctattactggagctggatccggcagcctcctggaaaaggattagaatggatcggcgagatagaccacagcgggagcacaaactacaaccccagcctgaaatcgcgggttacaatctctgtggacacaagcaagaatcagttctccctgaagctgagcagcgttactgccgccgacacagctgtgtactattgcgccagaggcggaggctcctggtacagcaactggttcgatccttggggccaaggcaccatggtgaccgtttccagcggctctacaagcggcagcgggaaacctggttctggagagggcagcacaaagggcgacatccagatgacacagagccccagcacccttagcgcctctgtgggagatagggttaccattacctgcagggcttcccagagcatcagcagctggctggcatggtatcaacagaagcctggcaaggctcccaagctgctcatctatgacgcctccagcctggaaagcggggttccctccagatttagcggctcaggctccggaacagagttcacccttaccatctctagcctgcaacccgacgacttcgctacttattactgtcaacaagacagaagcttgccccccacattcggcggagggaccaaggttgagatcaag

SEQ ID NO: 252 - DNA, 834 bp, synthetic construct
  source 1..834: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcagcggcatccactggaactggatccggcagcctcctggcaaaggccttgaatggatcggcgatatcgacaccagcggctccaccaactacaaccccagcctgaaatcgagggttacaatctctgtggacacaagcaagaatcagttctccctgaagctgagcagcgttactgccgccgacacagctgtgtactattgcgccagactgggccaggaaagcgctacctaccttggcatggatgtgtgggggcagggcaccaccgttactgttagctctggctcaacaagcggcagcggcaagcctggctcaggagaaggaagcacaaagggcgacattgtaatgactcagagccccgacagcctggccgttagcttaggcgaaagggctacaatcaattgcaagagcagccagagcgttctgtacagcagcaacaacaagaactacctcgcatggtatcaacagaagccaggccagcctcccaagctgctcatctactgggcttccaccagagagagcggggttcccgatagattctccggctccggttctggaacagatttcacgctcacaatcagcagcttacaggccgaggatgtggctgtctactattgtcagcagttgtacacctaccccttcacattcggcggaggcaccaaggttgagatcaag

SEQ ID NO: 253 - DNA, 825 bp, synthetic construct
  source 1..825: mol_type = other DNA
ggtacccccgggcccatggctcttcctgtgacagctcttctgctgcccctggccctgcttctgcatgctgctagacctcagcttcagctccaagagagcggacctggcttagtgaagcccagcgaaaccctgtccctcacctgcaccgtttctggcggaagcatcagcagctccagctattactggggatggatcaggcagccccctggcaagggtttagaatggatcggctcgatatattactccggcagcacctactataaccccagcttgaagagccgggttaccatttctgtggacacatcaaagaaccagttcagcctgaagctgagctctgtgactgccgccgacacagctgtgtactactgtgccagagagacagactactccagcggcatgggctacggcatggatgtgtggggacaaggaaccaccgttactgtgagcagcggttccaccagcggctcaggcaagcctggctcaggagaaggaagcaccaagggggatatacagatgacacagagcccctccagcctgtccgccagcgttggcgatcgtgtaacgatcacctgccgggcctctcagagcatcaactcctacctcaattggtatcaacagaagccaggcaaggcccccaaattactcatctacgccgccagcagcttacagagcggggttccctctagattctccggctccggttctggaacagatttcaccctcactatctccagcttgcagcccgaggatttcgccacttattactgtcagcagagcctggccgaccccttcacattcggcggaggcacaaaggttgagatcaag

SEQ ID NO: 254 - DNA, 828 bp, synthetic construct
  source 1..828: mol_type = other DNA
ggtacccccgggcccatggctcttcctgtgacagctcttctgctgcccctggccctgcttctgcatgctgctagacctcaggttcagcttgtgcagagcggagctgaagttaagaagcctggcgcctctgtgaaggttagctgcaaggccagcggctacacattcaaggaatatggcatctcctgggttaggcaggctcccggccaaggcttagaatggatgggctggatctccgcctactccggccacacctactacgcccagaagcttcagggcagggttaccatgaccaccgacaccagcacctctaccgcctatatggagctgaggagcctgagatcggacgacacagctgtgtattactgcgccagaggcccccactacgacgactggtctggatttatcatctggttcgacccctgggggcagggcaccctggtcacagtttcttctggctccaccagcggaagcggcaagccaggctcaggcgaaggatctacaaaaggcgacatccaaatgacacagagccccagcagcttgagcgcctccgttggcgacagagttacaatcacctgcagggcctctcagagcatcagcagctatttgaattggtatcaacagaagccaggaaaggcccctaagctgctcatctacgctgccagctcgctccaatctggcgttcctagcagatttagcggctccggcagcggcacagactttactcttaccattagctccctgcagcccgaggacttcgctacctactattgccagcaaagctacagattccctcccacctttggccagggcacaaaggttgagatcaag

SEQ ID NO: 255 - DNA, 825 bp, synthetic construct
  source 1..825: mol_type = other DNA
ggtacccccgggcccatggctcttcctgtgacagctcttctgctgcccctggccctgcttctgcatgctgctagacctcaggttcagttacaagagagcggacctggcttagtgaagcccagcgaaaccctgtccctcacctgcaccgtttctggcggaagcatcagctctcccgaccattactggggatggatcaggcagccccctggcaagggtttggaatggatcggcagcatctacgccagcggcagcacattctacaacccctcgctcaaaagcagggttactatttctgtggacacaagcaaaaatcagttcagcctgaagctgagctctgtgactgccgccgacacagctgtgtactactgtgccagagagacagactactccagcgggatgggctacggcatggatgtgtggggacaaggaaccaccgttactgtgagcagcggctccacaagcggctcaggcaagcctggctcaggagaaggaagcaccaagggggacattcaaatgacccaaagcccctccagcctgtccgccagcgttggcgatagggttaccattacctgcagggccagccaaagcatcaactcctacctaaattggtatcaacagaagccaggcaaggcccccaaactactcatttacgccgccagcagcttacagagcggggttccctctagattctccggcagcggttctggaacagatttcactctcacaatatcttcgctgcagcccgaggatttcgctacctactattgccagcaatccctggccgaccccttcacattcggcggaggcacaaaggttgagatcaag

SEQ ID NO: 256 - DNA, 822 bp, synthetic construct
  source 1..822: mol_type = other DNA
ggtacccccgggcccatggctcttcctgtgacagctcttctgctgcccctggccctgcttctgcatgctgctagacctcagatcacattaaaagagagcggacctacactggtgaagcccacccaaacgcttaccctcacctgcacctttagcgggttcagcctggacacagagggcgttggcgttggatggatcaggcagcctcctggcaaagccctcgaatggcttgccctcatctacttcaacgaccagaagagatacagcccctccttaaaatctcggctcacaatcaccaaagacacaagcaaaaatcaggttgtgctcaccatgaccaacatggaccctgtggacaccgctgtgtactactgtgccagagacaccggctacagcagatggtactacgggatggacgtttggggccaaggcaccactgtgaccgtttccagcggctctacaagcggcagcgggaaacctggttctggagagggcagcacaaagggcgacatccagatgacgcaatcccccagctctgtgagcgcctctgtgggagacagagttacaatcacatgccgggcctcccagggcatcagctcttggctggcatggtatcaacagaagcctggcaaggctcccaagctgctcatctatgccgcctcctccttacaatctggagttccctccaggttcagcgggagcggctcaggaacagacttcacccttaccatctctagcctgcaacccgaggacttcgctacttattactgtcagcaggcctacgcctaccccatcacattcggcggaggaacaaaggttgagatcaag

SEQ ID NO: 257 - DNA, 852 bp, synthetic construct
  source 1..852: mol_type = other DNA
ggtacccccgggcccatggctcttcctgtgacagctcttctgctgcccctggccctgcttctgcatgctgctagacctcaggttcagttgcagcaatggggagctggcctgttaaagcccagcgaaaccctgtccctcacctgcgctgtgtatggcggaagcttcgagaaatactactggagctggatccggcagcctcccggcaaaggcttagaatggatcggcgagatttatcacagcgggctcaccaactacaaccccagcctgaaatctcgagttacaatctctgtggacacaagcaagaatcagttctccctgaagctgagcagcgttactgccgccgacacagctgtgtactattgcgccagagttagatacgacagcagcgacagctattactacagctatgactacggcatggatgtgtgggggcagggcaccaccgttactgtctcctctggatctaccagcggcagcggcaagcctggatctggcgaaggaagcacaaagggcgacattgtgctcacccagagccccgacagcctggctgtgtctttaggcgaaagggctaccatcaactgcaagagcagccagagcgttctgtacagcagcaacaacaagaactaccttgcttggtatcaacagaagcctggccagccccctaagctgctcatctactgggcctctagcagagagagcggggttcccgatcggtttagcggctccggctcaggaaccgatttcaccctcactatctccagcctccaggccgaggatgtggctgtctactattgtcagcagagctatagcttcccctggacattcggcggaggcaccaaggttgagatcaag

SEQ ID NO: 258 - DNA, 852 bp, synthetic construct
  source 1..852: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttcagccgctatgtgtggagctggatccggcagcctcctggcaaaggccttgaatggatcggagagatagacagcagcggcaagaccaactacaaccccagcctgaaatcacgcgttacaatctctgtggacacaagcaagaatcagttctccctgaagctgagcagcgttactgccgccgacacagctgtgtactattgcgccagagttagatacgacagctccgacagctattactacagctatgactacggcatggatgtgtgggggcagggcaccaccgttacagttagctctggaagcaccagcggctccggcaagcctggatctggtgaaggaagcacaaagggcgacattgtgctcacccagagccccgacagcctggctgtgtctttaggcgaaagggctaccatcaactgcaagagcagccagagcgttctgtacagcagcaacaacaagaactaccttgcatggtatcaacagaagcctggccagcctcccaagctgctcatctactgggcctctagcagagagagcggggttcccgatcgctttagcggcagcggttctggcaccgatttcactcttacaatcagcagcttacaggccgaggatgtggctgtctactattgtcagcagagctatagcttcccctggacattcggcggaggcaccaaggttgagatcaag

SEQ ID NO: 259 - DNA, 852 bp, synthetic construct
  source 1..852: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttcagcggctacgcttggagctggattagacagcctcctggcaaaggactagaatggatcggagagatcgaccacagaggcttcaccaactacaaccccagcctgaaatccagagttacaatctctgtggacacaagcaagaatcagttctccctgaagctgagcagcgttactgccgccgacacagctgtgtactattgcgccagggttagatacgacagcagcgacagctattactacagctatgactacggcatggatgtgtgggggcagggcaccaccgttacggttagctctggatctaccagcggcagcggcaagcctggctcaggagaaggaagcacaaagggcgacattgtgctcacccagagccccgacagcctggccgtttctttaggcgaaagggctaccatcaactgcaagagcagccagagcgttctgtacagcagcaacaacaagaactaccttgcatggtatcaacagaagccaggccagcctcccaagctgctcatctactgggcctctagcagagagagcggggttcccgatagattttcgggatcaggctccggcaccgatttcactcttacgatcagcagcttacaggccgaggatgtggctgtctactattgtcagcagagctatagcttcccctggacattcggcggaggcaccaaggttgagatcaag

SEQ ID NO: 260 - DNA, 840 bp, synthetic construct
  source 1..840: mol_type = other DNA
ggtacccccgggcccatggctcttcctgtgacagctcttctgctgcccctggccctgcttctgcatgctgctagacctcaggttcagttacaacaatggggagctggcctgttaaagcccagcgaaaccctgtccctcacctgcgctgtgtatggcggaagcttccagaaatactactggagctggatccggcagcctcccggcaaaggcttagaatggatcggagagatagacaccagcggcttcaccaactacaaccccagcctgaaatctagggttacaatctctgtggacacaagcaagaatcagttctccctgaagctgagcagcgttactgccgccgacacagctgtgtactattgcgccagagttggcagatacagctacggctactacatcaccgccttcgacatttggggccaaggcaccactgtgaccgtttccagcggaagcactagcggcagcgggaaacctggttctggagagggctcaaccaagggcgacatcgtgatgacacagagccccgactctctggctgtgtccctgggagagagagccaccatcaactgcaagagcagccagagcgttctgtacagcagcaacaacaagaactacctggcatggtatcaacagaagcctggccagccccctaagctgctcatctactgggcttccaccagagaatcaggcgttccagacaggttctccggctcgggttcaggcacagacttcacccttaccatctcttccctgcaggccgaagatgtggccgtttactactgtcagcagcactacagcttccctttcacattcggcggaggcaccaaggttgagatcaag

SEQ ID NO: 261 - DNA, 258 bp, synthetic construct
  source 1..258: mol_type = other DNA
gcagctgctttcgtgcctgtgttcctgcctgctaagcccaccaccactcctgctccaagacctcctacccccgctcctacaatcgccagccaacctctgagcctgagaccggaggcatgcagacctgcggcagggggagcagttcacacaagaggcttggacttcgcttgcgacatctacatctgggcccctctggccggcacatgcggagttcttcttcttagcctggtgatcaccctgtactgcaaccacagaaac

SEQ ID NO: 262 - DNA, 180 bp, synthetic construct
  source 1..180: mol_type = other DNA
gctgctgcattggataatgaaaaatcgaacggcacaatcattcatgtgaagggcaaacacctgtgtcccagccccttgttcccaggacctagcaagcctttttgggttctcgtggtggtgggcggcgttctggcttgctactctctacttgtaactgtcgcatttattatattctgggtt

SEQ ID NO: 263 - DNA, 186 bp, synthetic construct
  source 1..186: mol_type = other DNA
ggaggaggaggatctctggataacgagaaaagcaacgggaccatcattcatgtgaagggaaaacatctgtgtcccagccccttgttccccggacctagcaagccgttttgggttctcgtggtggtgggcggcgttctggcttgctactctctgcttgtgaccgttgccttcattatcttctgggtt

SEQ ID NO: 264 - DNA, 201 bp, synthetic construct
  source 1..201: mol_type = other DNA
ggaggaggaggatctggtggaggaggttctctggacaatgagaaatcaaatggaacgatcatccatgtgaaggggaagcacctctgcccctctcccctgtttcctggtcctagcaagcccttctgggttttggtggtcgtgggcggcgttctggcttgctacagcctgttagtgaccgttgcatttatcatattttgggtt

SEQ ID NO: 265 - DNA, 201 bp, synthetic construct
  source 1..201: mol_type = other DNA
ggaggaggaggatctggtggaggaggttctctggacaatgagaaatcgaatgggacaatcatccatgtgaaggggaagcacctgagcccctctcccctgtttcctggtcctagcaagcccttctgggttttggtggtcgtgggcggcgttctggccgtttacagcctgttagtgaccgttgcttttatcatattttgggtt

SEQ ID NO: 266 - DNA, 201 bp, synthetic construct
  source 1..201: mol_type = other DNA
ggaggaggaggatctggtggaggaggttctctggacaatgaaaagagcaatggcacaatcatccatgtgaaggggaagcacctgaacggctccgccctgtttcctggtcctagcaagccattttgggttctcgtggtggtgggcggcgttctggccgtttacagcctgttagtgaccgttgcgttcataatcttctgggtt

SEQ ID NO: 267 - DNA, 216 bp, synthetic construct
  source 1..216: mol_type = other DNA
ggaggaggaggatctggtggaggaggttctggaggaggcggctctctcgacaacgaaaagagcaatggcaccattattcacgttaaaggcaagcatctgtgcccctcccccctgttccccggaccttcaaaacctttttgggttctcgtggtggtgggcggcgttctggcctgctattctttgctggtaactgtagccttcattatcttctgggtt

SEQ ID NO: 268 - DNA, 771 bp, synthetic construct
  source 1..771: mol_type = other DNA
gagagcaagtacggacctccttgtcctccatgtcctgctcccgagttcgagggcggaccttcagtgttcctgttcccccctaaacccaaggatactcttatgatcagccggacccccgaggtcacctgtgtggtggtagatgttagccaggaggatcccgaggtgcagttcaactggtacgtcgacggcgtcgaggtacacaacgccaagaccaagcctagggaggagcagttccagtccacctatagggtcgtgagcgtgcttaccgtgctgcaccaggactggttgaacggcaaggagtacaagtgcaaggtgtccaacaagggcctccccagcagcatcgagaagaccattagcaaggcaaagggacagcccagggagccccaggtgtacacattacctccttcccaggaagagatgaccaagaaccaggtgtcgcttacctgcctggtcaagggcttctacccctccgacattgcagttgaatgggagtcaaacggccagccggagaacaattacaagaccacccccccagtcttggacagcgacggctctttcttcctctactcgcggcttactgtagataaaagtcgttggcaggagggaaacgtgttcagctgctctgtgatgcacgaggccctccataaccactacacccagaagagcctctccctgtctctgggcaagatgttctgggtgctggtcgtggtgggcggagttcttgcttgctactccctgctcgtgaccgtcgctttcattatattctgggtc

SEQ ID NO: 269 - DNA, 795 bp, synthetic construct
  source 1..795: mol_type = other DNA
gagagaaagtgttgtgttgagtgtcctccttgtcctccctgccctgctcccgagttacttggcggaccttcagtgttcctgttcccccccaagcccaaggatactctcatgatcagccggacccccgaggtcacctgtgtggtggtagatgttagccacgaggaccctgaggtcaagttcaactggtacgtcgacggcgtcgaggtgcacaacgccaagaccaagcctcgtgaagaacagtaccagtccacctacagagttgtgagcgtgcttaccgtgctgcaccaggactggctgaacggcaaggagtacaagtgcaaggtgtccaacaaggccctccccgctcccatcgagaagacaatcagcaaggccaagccctgtccagcccctgagctcttaggaggacctagcgttttccttttccctcccaagcctaaggacactcttatgatctccagaacaccagaggttacctgcgtcgtggtggacgtgtcccatgaggacccagaagtcaaattcaattggtatgtagatggggtcgaggtccacaacgctaagacaaagccccgcgaggagcagtacaactctacctacagggtcgtgtccgtgctcacagtgctgcatcaggattggctcaacgggaaggagtataagtgcaaagtgtccaataaggcccttcccgcccctatcgagaaaaccatctctaaggccaaattctgggtgctggtggttgtgggcggcgtgcttgcttgttactccctgctggtcactgtagctttcatcatattttgggtg

SEQ ID NO: 270 - protein, 40 aa, synthetic construct
RGRKKLLYIFKQPFMRPVQTTQEEDGCSCRFPEEEEGGCE

SEQ ID NO: 271 - protein, 495 aa, synthetic construct
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSSGSTSGSGKPGSGEGSTKGDIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIKAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 272 - protein, 467 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 273 - protein, 39 aa, synthetic construct
IEVMYPPPYLDNEKSNGTIIHVKGKHLCPSPLFPGPSKP

SEQ ID NO: 274 - protein, 30 aa, synthetic construct
LDNEKSNGTIIHVKGKHLCPSPLFPGPSKP

SEQ ID NO: 275 - protein, 45 aa, synthetic construct
TTTPAPRPPTPAPTIASQPLSLRPEACRPAAGGAVHTRGLDFACD

SEQ ID NO: 276 - protein, 27 aa, synthetic construct
FWVLVVVGGVLACYSLLVTVAFIIFWV

SEQ ID NO: 277 - protein, 24 aa, synthetic construct
IYIWAPLAGTCGVLLLSLVITLYC

SEQ ID NO: 278 - protein, 111 aa, synthetic construct
VKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 279 - protein, 41 aa, synthetic construct
RSKRSRLLHSDYMNMTPRRPGPTRKHYQPYAPPRDFAAYRS

SEQ ID NO: 280 - protein, 22 aa, synthetic construct
MLLLVTSLLLCELPHPAFLLIP

SEQ ID NO: 281-292 - skipped (12 entries)

SEQ ID NO: 293 - protein, 5 aa, synthetic construct
GGGGS

SEQ ID NO: 294 - protein, 10 aa, synthetic construct
GGGGSGGGGS

SEQ ID NO: 295 - skipped

SEQ ID NO: 296 - protein, 15 aa, synthetic construct
GGGGSGGGGSGGGGS

SEQ ID NO: 297 - protein, 20 aa, synthetic construct
GGGGSGGGGSGGGGSGGGGS

SEQ ID NO: 298 - protein, 18 aa, synthetic construct
PSTPPTPSPSTPPTPSPS

SEQ ID NO: 299 - protein, 50 aa, synthetic construct
  VARIANT 1..50: note = This sequence may encompass 1-10 PAPAP repeating units
PAPAPPAPAPPAPAPPAPAPPAPAPPAPAPPAPAPPAPAPPAPAPPAPAP

SEQ ID NO: 300 - protein, 50 aa, synthetic construct
  VARIANT 1..50: note = This sequence may encompass 1-10 EAAAK repeating units
EAAAKEAAAKEAAAKEAAAKEAAAKEAAAKEAAAKEAAAKEAAAKEAAAK

SEQ ID NO: 301 - protein, 8 aa, synthetic construct
  VARIANT 2: note = Valine or Isoleucine
  VARIANT 4: note = Any amino acid
DXEXNPGP

SEQ ID NO: 302 - protein, 50 aa, synthetic construct
  VARIANT 1..5: note = This region may encompass 1-5 residues
  VARIANT 6..10: note = This region may encompass 1-5 residues
  VARIANT 1..50: note = This sequence may encompass 1 to 5 or more repeating units of GxSx, wherein x=1-5
GGGGGSSSSSGGGGGSSSSSGGGGGSSSSSGGGGGSSSSSGGGGGSSSSS